(19) United States
(12) Patent Application Publication
(10) Pub. No.: US 2026/0317443 A1
(43) Pub. Date: Oct. 15, 2026

(54) VON WILLEBRAND FACTOR (VWF) ANTIBODY

(71) Applicant: IP2IPO INNOVATIONS LTD, London (GB)

(72) Inventors: Nadine Clemo, Cambridge (GB); Tom McKinnon, London (GB)

(21) Appl. No.: 19/111,031

(22) PCT Filed: Oct. 19, 2023
(86) PCT No.: PCT/GB2023/052711

§ 371 Date: Mar. 12, 2025

(30) Foreign Application Priority Data
Oct. 19, 2022 (GB) 2215432.2

(51) Int. Cl. C07K 16/36; A61P 7/02; G01N 33/86

(52) U.S. Cl. CPC C07K 16/36; A61P 7/02; G01N 33/86; C07K 2317/14; C07K 2317/24; C07K 2317/52; C07K 2317/565; G01N 2333/755; G01N 2800/224; G01N 2800/52

(57) ABSTRACT

The invention relates to Von Willebrand Factor (VWF) antibodies, and particularly, although not exclusively, to antibodies which target the C1-C6 domain of VWF. The invention extends to compositions comprising the antibodies, including pharmaceutical compositions and kits. The invention also extends to methods of using the antibodies, for example in therapy and diagnosis of conditions caused by platelet-mediated aggregation, including various cardiovascular diseases, such as acquired thrombotic thrombocytopenic purpura (aTTP), ischemic stroke and atherosclerosis.

The present invention relates to Von Willebrand Factor (VWF) antibodies, and particularly, although not exclusively, to antibodies which target the C1-C6 domain of VWF. The invention extends to compositions comprising the antibodies, including pharmaceutical compositions and kits. The invention also extends to methods of using the antibodies, for example in therapy and diagnosis of conditions caused by platelet-mediated aggregation, including various cardiovascular diseases, such as acquired thrombotic thrombocytopeniarpura (aTTP), ischemic stroke and atherosclerosis.

Cardiovascular diseases (CVDs), including ischemic heart disease, stroke, heart failure, peripheral arterial disease, and a number of other cardiac and vascular conditions, remain the leading cause of death globally. CVDs are characterised by thrombotic events, caused by uncontrolled platelet aggregation, that contribute to both cell death and organ failure. Given the central role of platelets in triggering thrombosis, several approved antithrombotic drugs target platelets, such as aspirin, clopidogrel, and abciximab. Due to their complementary mechanisms of action, the combination of these agents inhibits platelet aggregation to a greater extent than any of the agents acting alone. However, the use of these antiplatelet drugs is hampered by an increased bleeding risk, reducing their application in wider patient populations. Therefore, there remains a high unmet medical need for therapies that can treat thrombotic disorders without the severe risk of bleeding.

Von Willebrand Factor (VWF), a large multimeric glycoprotein, present in blood plasma, endothelial cells, megakaryocytes, and platelets, is a well-established risk factor of thrombotic events. VWF plays a major role in haemostasis, mediating platelet adhesion to vascular injury sites, and protecting coagulation factor VIII (FVIII) from degradation. Following injury, collagen is exposed in the damaged vessel wall to flowing blood and shear forces. Plasma VWF binds to the exposed collagen and uncoils its structure, supporting the adhesion of circulating platelets. The bound VWF then interacts with the platelet receptor GPIb and platelet tethering occurs. Platelet plug formation is achieved once a critical mass of platelets, VWF and associated coagulation proteins bind together to seal the vessel wall. Other functions have also been reported for VWF, including regulation of inflammation and angiogenesis.

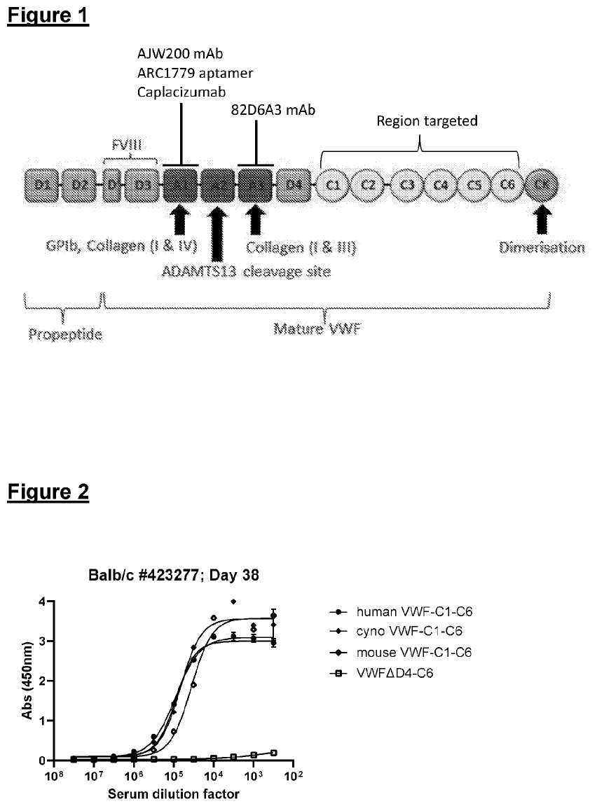

The accumulation of VWF has been associated with increased risk to CVDs. In particular, the accumulation of ultra-long VWF (ULvWF) in thrombotic thrombocytopenia purpura (TTP) patients, has been widely studied. Currently, Caplacizumab (a monoclonal antibody) is the only approved anti-VWF therapy, which has been shown to block the binding of VWF to platelets and reduce thrombi formation in TTP patients. However, this antibody functions by targeting and inhibiting the A1 domain of VWF (see FIG. 1). The A1 domain is essential for collagen binding, and therefore platelet binding, under low shear conditions, for normal haemostasis to take place. As such, by targeting this region, treatment with Caplacizumab results in a severe bleeding risk in patients. For patients suffering from TTP, a rare and fatal blood clotting disorder, the benefit of taking Caplacizumab outweighs the risk of severe bleeding. However, this severe bleeding risk is not acceptable for patients suffering from other CVDs, including ischemic stroke and myocardial infarction. Therefore, there exists a significant unmet medical need for new antithrombotic therapies that can be used to treat a wider range of thrombotic disorders, without the risk of severe bleeding.

The inventors have identified that a previously untargeted region of VWF, within the C1-C6 domain (see FIG. 1), is critical for VWF to enable platelets to clot under high blood shear rates, such as those found in thrombotic conditions. Importantly, the C1-C6 domain is not essential for platelet binding under low shear conditions (i.e. normal bleeding). Accordingly, this identifies the C1-C6 domain of VWF as a potential new therapeutic target for the treatment of a number of conditions caused by platelet-mediated aggregation or thrombotic-related conditions, including aTTP, ischemic stroke and atherosclerosis.

As such, the inventors hypothesised that by inhibiting the ability of the VWF C1-C6 region under high shear rates, they could reduce platelet clotting in thrombotic conditions. Importantly, by targeting this region, VWF retains its ability to bind to platelets as normal under low shear rates (via the A1 domain), for normal haemostasis to occur, and so does not suffer from the significant problems associated with using Caplacizumab. This has led to the inventors' further work in developing antibodies that are capable of targeting the C1-C6 domain of VWF to inhibit its pro-thrombotic function. These anti-VWF monoclonal antibodies may be used in the treatment, amelioration or prevention of a number of thrombotic-related conditions, and would be much safer than the currently available treatments, as there would be a reduced risk of severe bleeding.

Accordingly, in a first aspect of the invention, there is provided an antibody or antigen-binding fragment thereof that specifically binds to one or more of a C1, C2, C3, C4, C5, and/or C6 domain of Von Willebrand Factor (VWF).

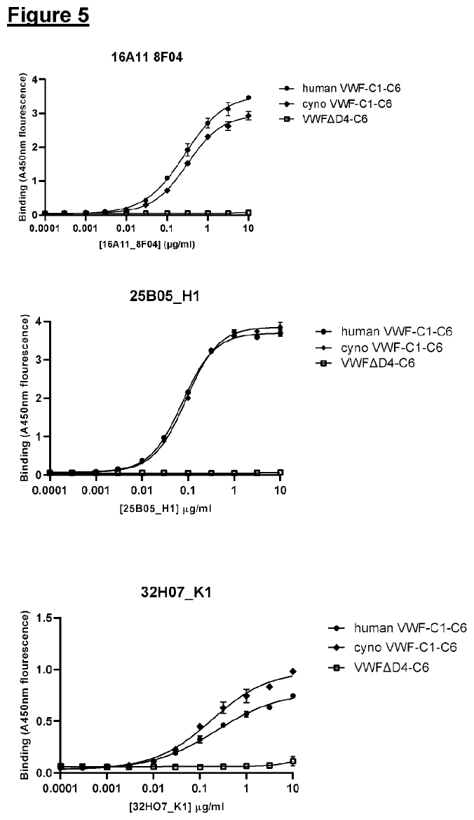

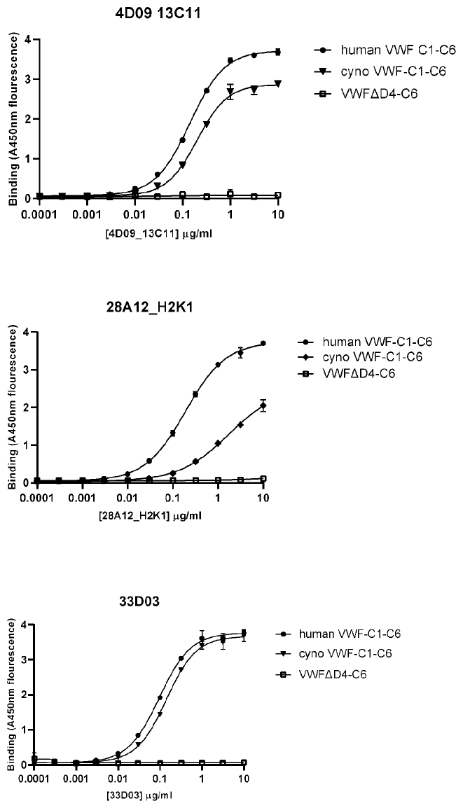

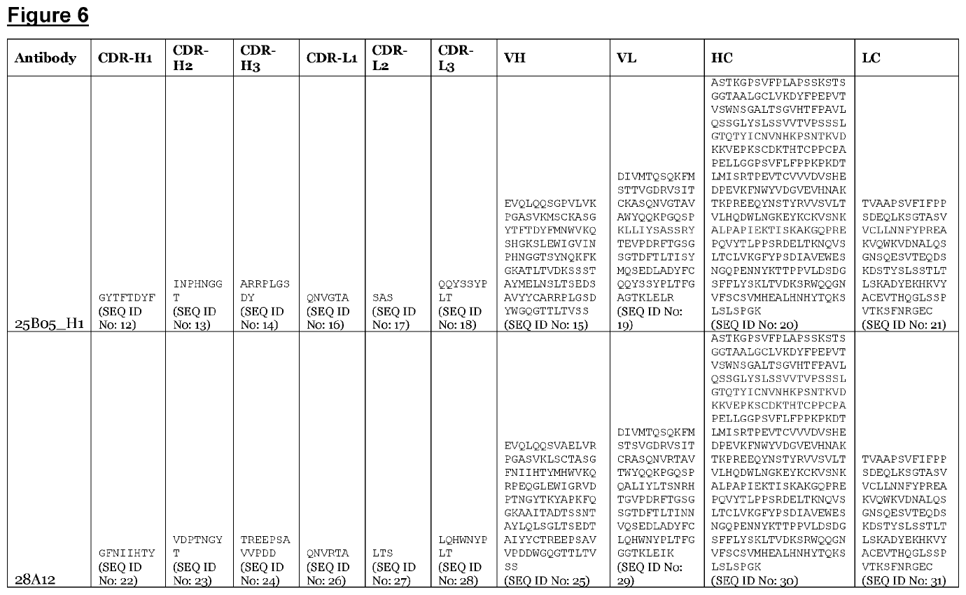

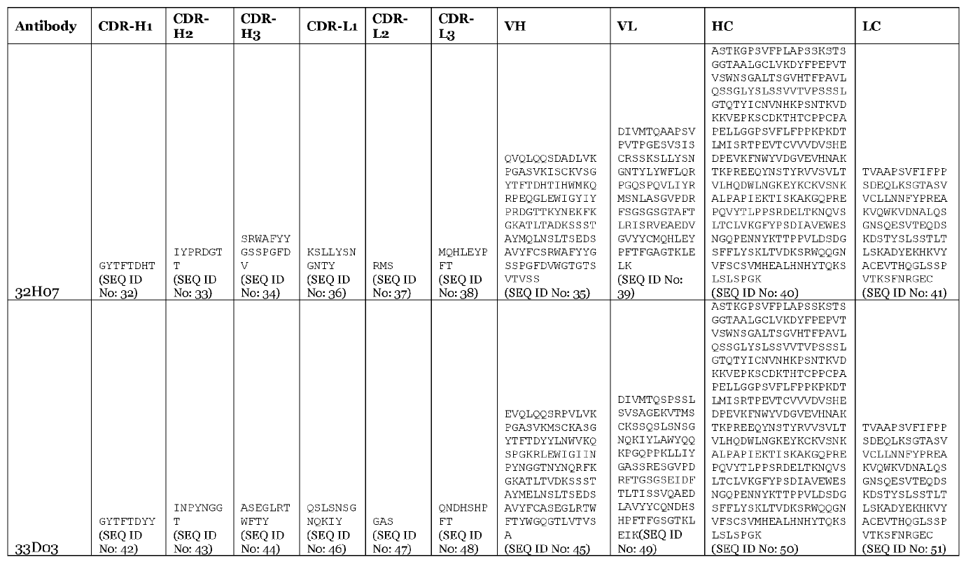

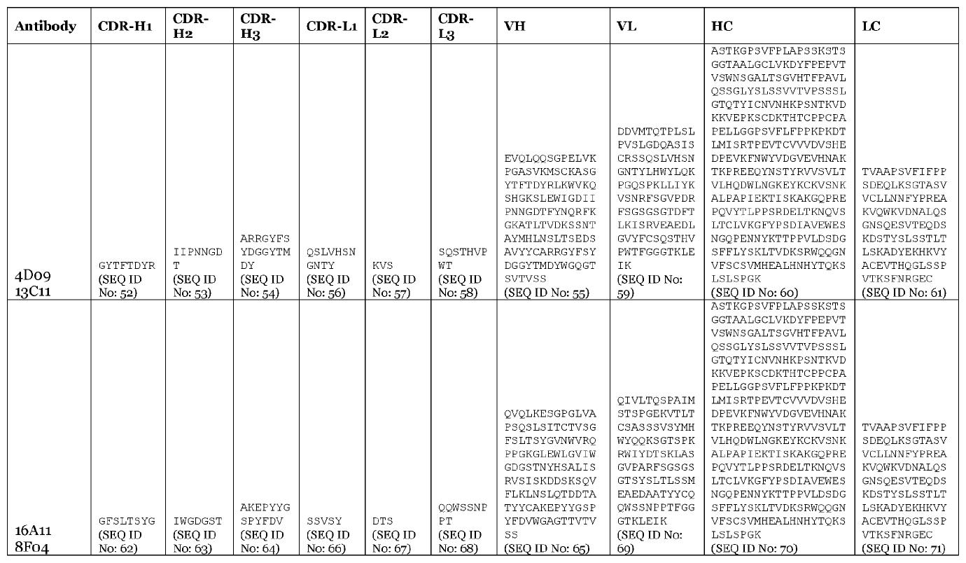

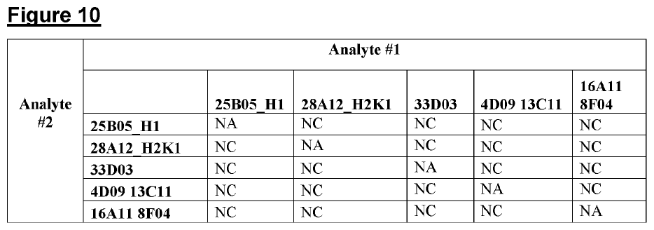

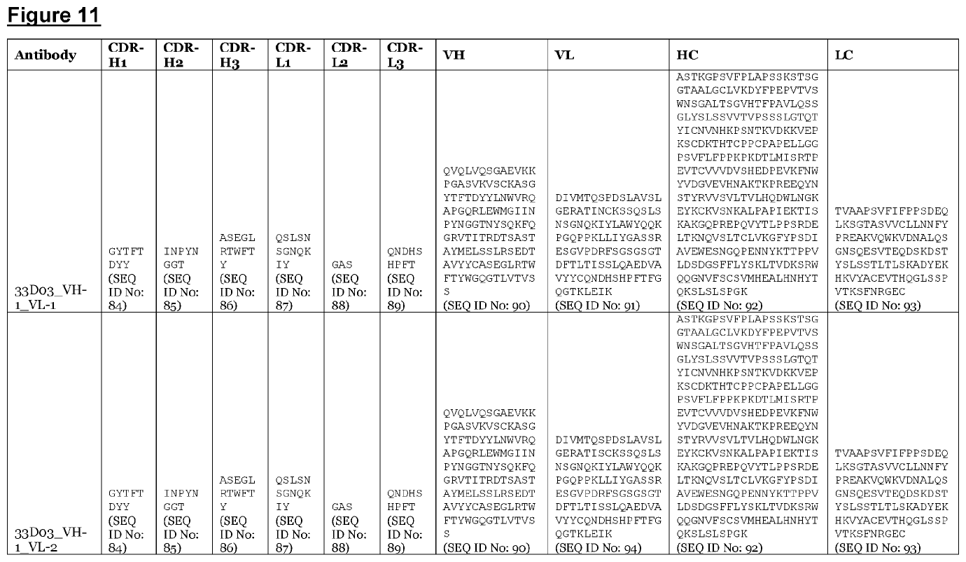

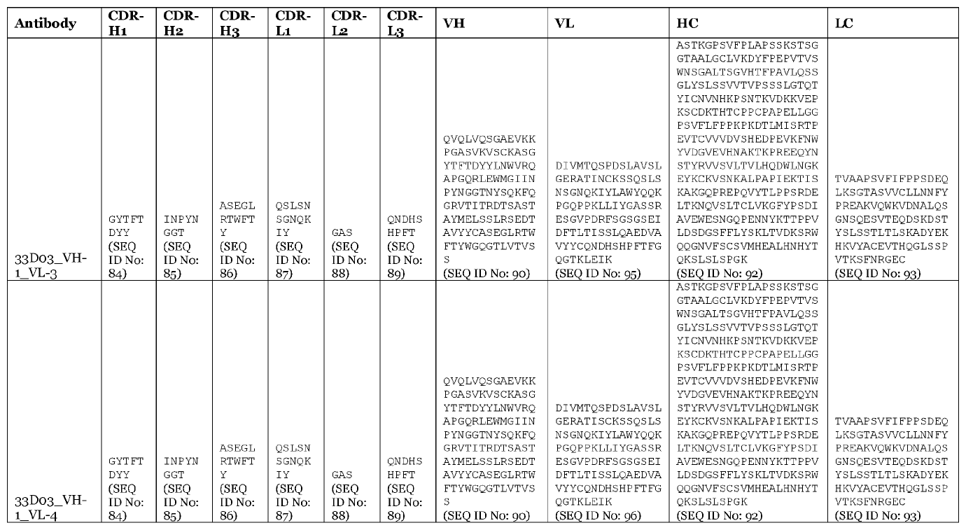

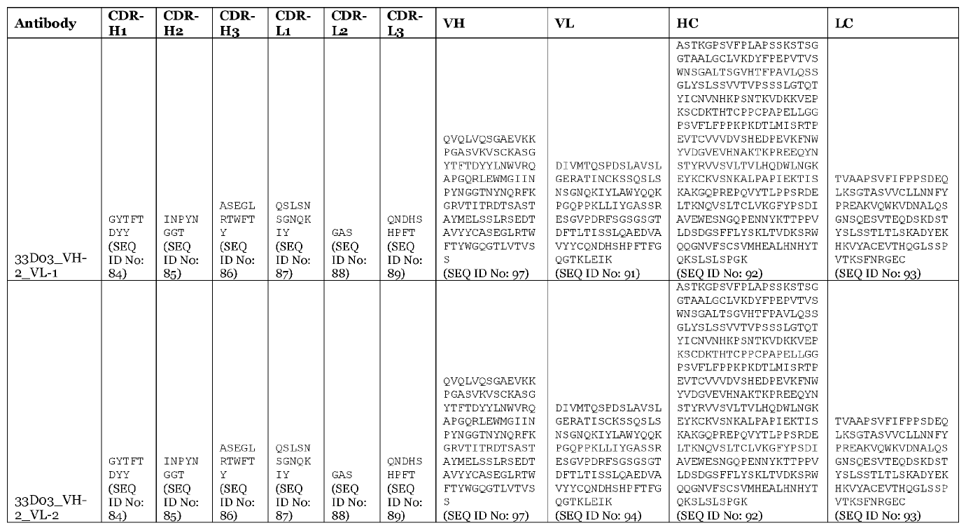

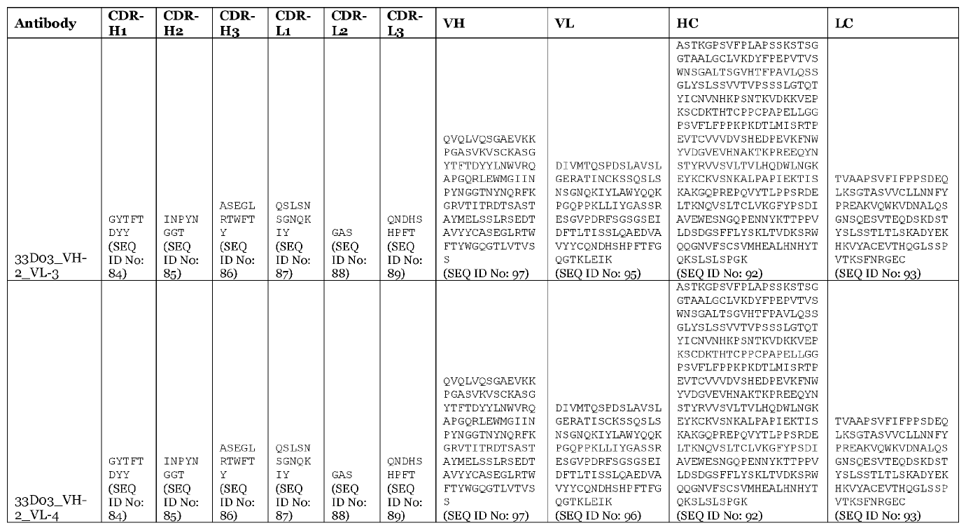

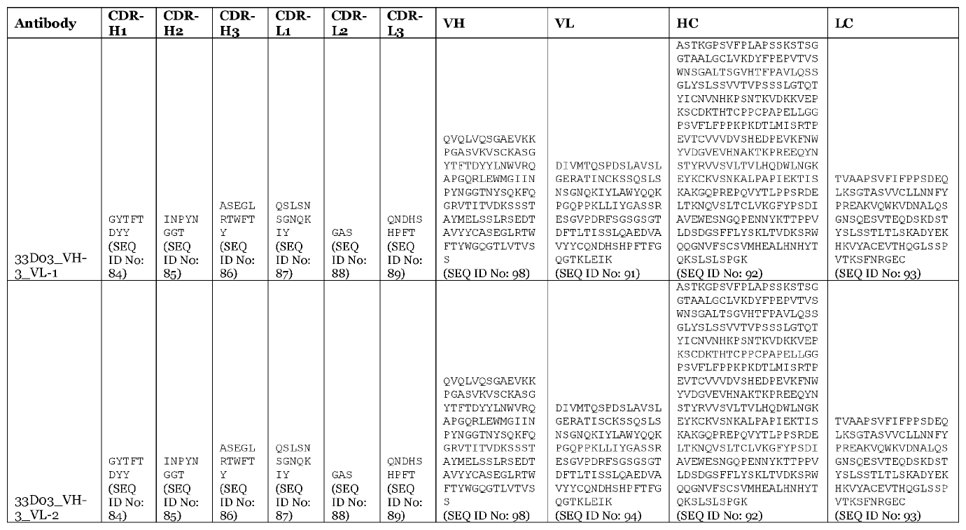

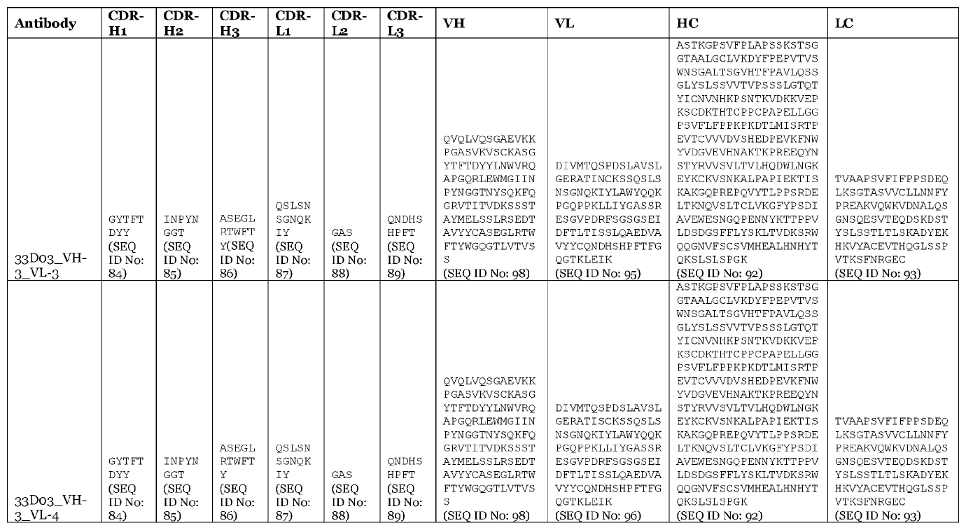

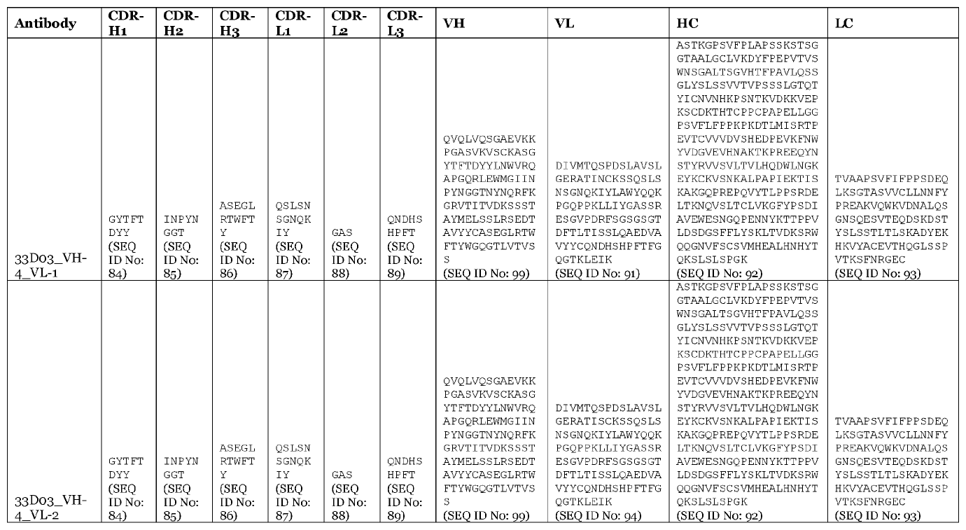

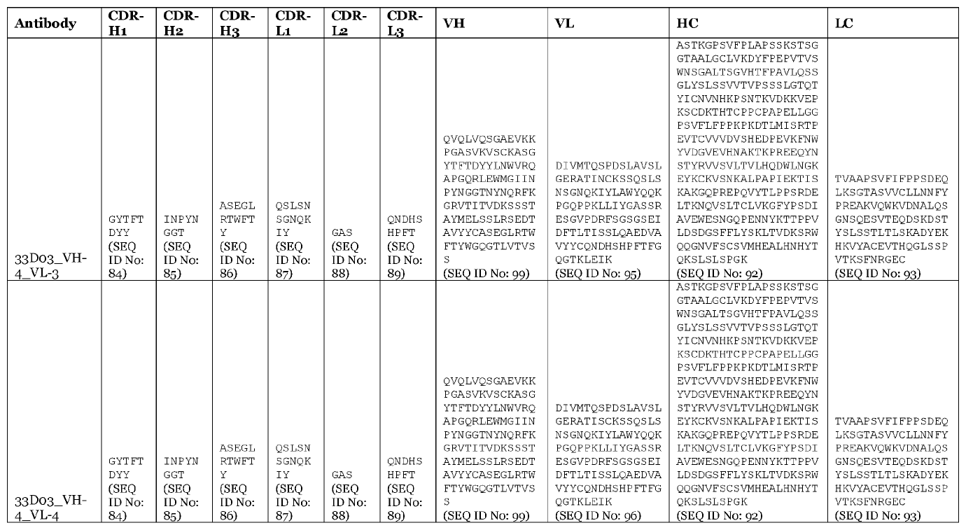

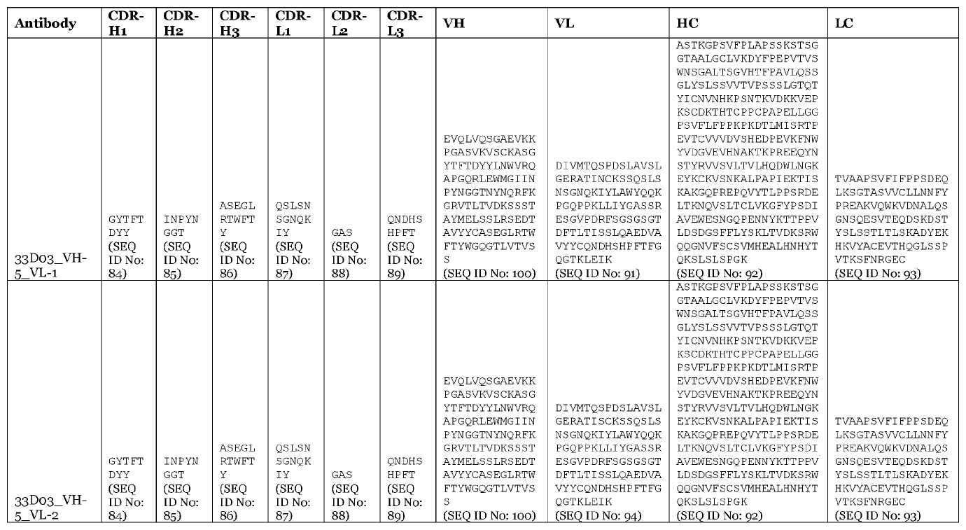

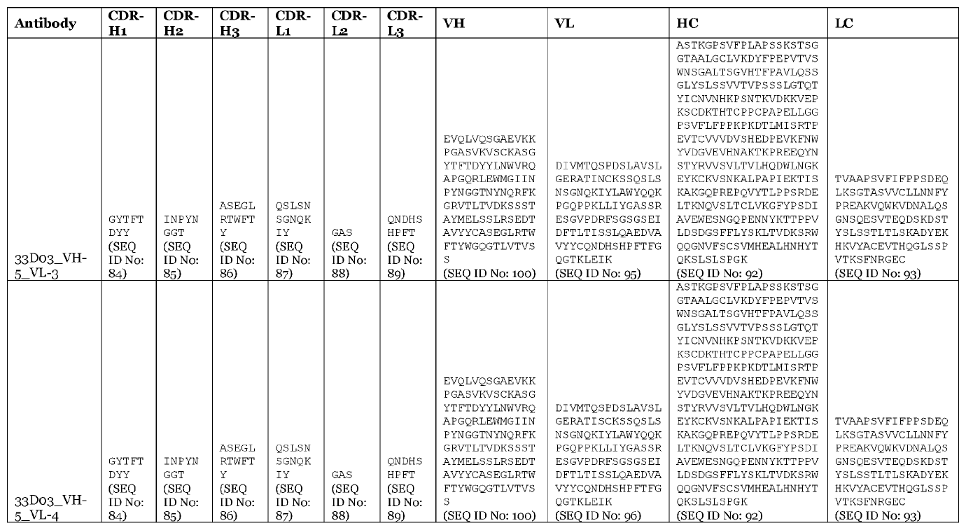

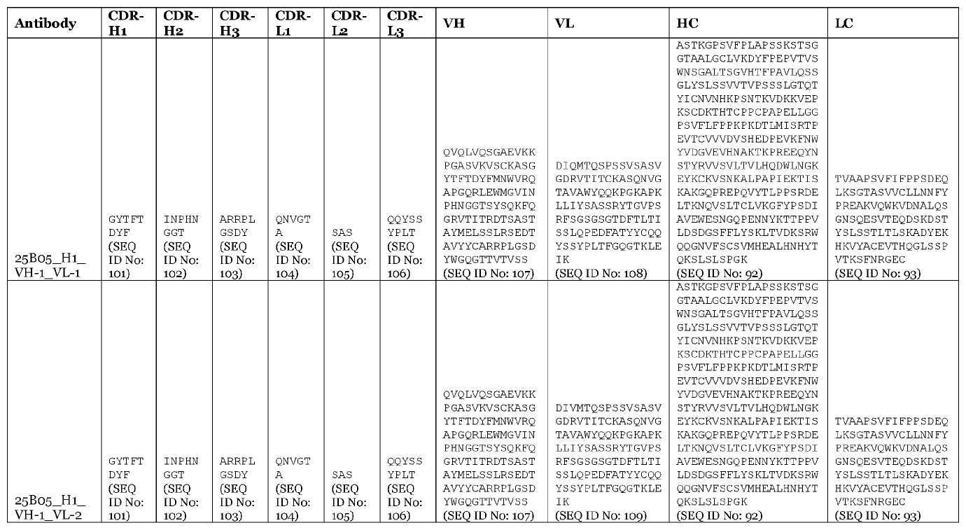

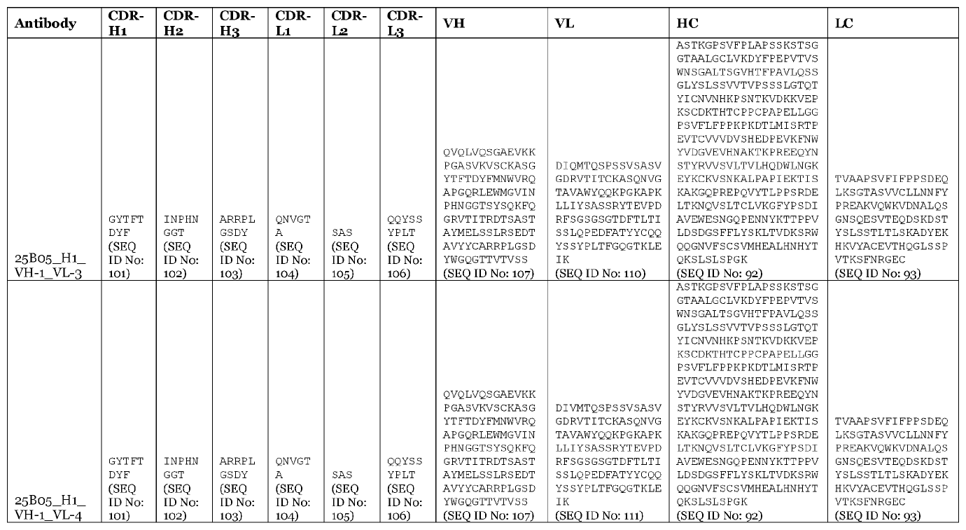

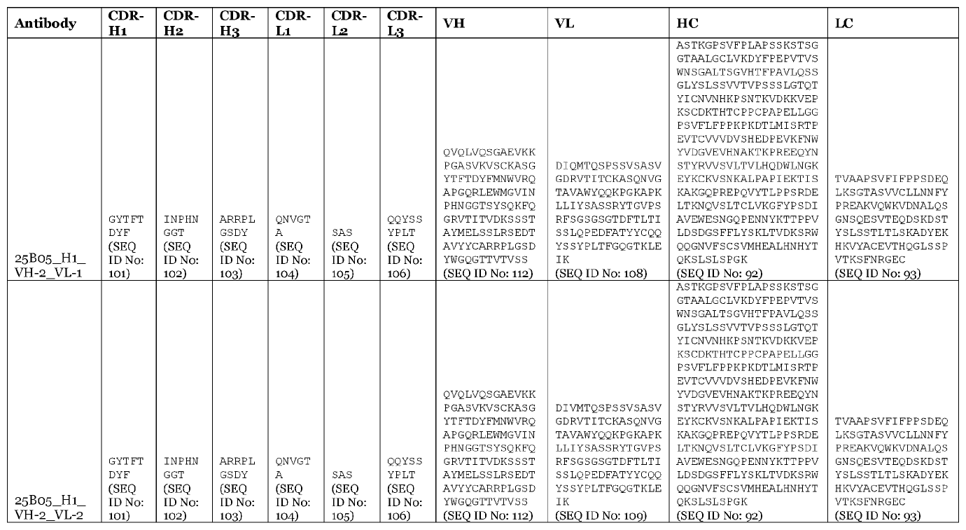

As shown in the examples, the inventors have developed a number of antibodies that are capable of binding to, and inhibiting the function of the C1-C6 domains of VWF. For example, as shown in FIGS. 4 and 5, the inventors have developed six antibodies that target within the C1-C6 domains of VWF. Advantageously, by targeting one or more of the C1-C6 domains of VWF, the antibodies according to the invention will inhibit the pro-thrombotic function of VWF, without inhibiting its normal haemostatic function. For example, as illustrated in FIG. 9, the antibodies inhibit platelet capture under high shear rate (5000 s⁻¹) to a greater extent than under normal shear rate conditions (1500 s⁻¹), indicating that the antibodies can block the pro-thrombotic function of VWF while maintaining its normal haemostatic function.

In one embodiment, preferably the antibody or antigen-binding fragment thereof specifically binds to the C1 domain of VWF. In another embodiment, preferably the antibody or antigen-binding fragment thereof specifically binds to the C2 domain of VWF. In another embodiment, preferably the antibody or antigen-binding fragment thereof specifically binds to the C3 domain of VWF. In another embodiment, preferably the antibody or antigen-binding fragment thereof specifically binds to the C4 domain of VWF. In another embodiment, preferably the antibody or antigen-binding fragment thereof specifically binds to the C5 domain of VWF. In another embodiment, preferably the antibody or antigen-binding fragment thereof specifically binds to the C6 domain of VWF.

Preferably, the antibody or antigen-binding fragment thereof is capable of inhibiting the function of one or more of a C1, C2, C3, C4, C5, and/or C6 domain of VWF. In a preferred embodiment, the antibody or antigen-binding fragment thereof is capable of inhibiting the function of the C5 domain of VWF. Preferably, the antibody or antigen-binding fragment thereof is capable of inhibiting the function of one or more of a C1, C2, C3, C4, C5, and/or C6 domain of VWF, such that platelet binding is inhibited under conditions of high shear rate, i.e. pathological conditions. Preferably, the antibody or antigen-binding fragment thereof is capable of inhibiting the function of one or more of a C1, C2, C3, C4, C5, and/or C6 domain of VWF, such that platelet binding is not inhibited under conditions of low shear rate, i.e. normal conditions.

In one embodiment, the amino acid sequence of VWF may be represented by Genbank ID No: NM_000552.5, which is provided herein as SEQ ID No: 1, as follows:

[SEQ ID No: 1]
MIPARFAGVLLALALILPGTLCAEGTRGRSSTARCSLFGSDFVNT
FDGSMYSFAGYCSYLLAGGCQKRSFSIIGDFQNGKRVSLSVYLGE
FFDIHLFVNGTVTQGDQRVSMPYASKGLYLETEAGYYKLSGEAYG
FVARIDGSGNFQVLLSDRYENKTCGLCGNFNIFAEDDEMTQEGTL
TSDPYDFANSWALSSGEQWCERASPPSSSCNISSGEMQKGLWEQC
QLLKSTSVEARCHPLVDPEPFVALCEKTLCECAGGLECACPALLE
YARTCAQEGMVLYGWTDHSACSPVCPAGMEYRQCVSPCARTCQSL
HINEMCQERCVDGCSCPEGQLLDEGLCVESTECPCVHSGKRYPPG
TSLSRDCNTCICRNSQWICSNEECPGECLVTGQSHFKSFDNRYFT
FSGICQYLLARDCQDHSFSIVIETVQCADDRDAVCTRSVTVRLPG
LHNSLVKLKHGAGVAMDGQDVQLPLLKGDLRIQHTVTASVRLSYG
EDLQMDWDGRGRLLVKLSPVYAGKTCGLCGNYNGNQGDDFLTPSG
LAEPRVEDFGNAWKLHGDCQDLQKQHSDPCALNPRMTRFSEEACA
VLTSPTFEACHRAVSPLPYLRNCRYDVCSCSDGRECLCGALASYA
AACAGRGVRVAWREPGRCELNCPKGQVYLQCGTPCNLTCRSLSYP
DEECNEACLEGCFCPPGLYMDERGDCVPKAQCPCYYDGEIFQPED
IFSDHHTMCYCEDGFMHCTMSGVPGSLLPDAVLSSPLSHRSKRSL
SCRPPMVKLVCPADNLRAEGLECTKTCQNYDLECMSMGCVSGCLC
PPGMVRHENRCVALERCPCFHQGKEYAPGETVKIGCNTCVCQDRK
WNCTDHVCDATCSTIGMAHYLTEDGLKYLFPGECQYVLVQDYCGS
NPGTFRILVGNKGCSHPSVKCKKRVTILVEGGEIELFDGEVNVKR
PMKDETHFEVVESGRYIILLLGKALSVVWDRHLSISVVLKQTYQE
KVCGLCGNEDGIQNNDLTSSNLQVEEDPVDFGNSWKVSSQCADTR
KVPLDSSPATCHNNIMKQTMVDSSCRILTSDVFQDCNKLVDPEPY
LDVCIYDTCSCESIGDCACFCDTIAAYAHVCAQHGKVVTWRTATL
CPQSCEERNLRENGYECEWRYNSCAPACQVTCQHPEPLACPVQCV
EGCHAHCPPGKILDELLQTCVDPEDCPVCEVAGRRFASGKKVTLN
PSDPEHCQICHCDVVNLTCEACQEPGGLVVPPTDAPVSPTTLYVE
DISEPPLHDFYCSRLLDLVFLLDGSSRLSEAEFEVLKAFVVDMME
RLRISQKWVRVAVVEYHDGSHAYIGLKDRKRPSELRRIASQVKYA
GSQVASTSEVLKYTLFQIFSKIDRPEASRITLLLMASQEPQRMSR
NFVRYVQGLKKKKVIVIPVGIGPHANLKQIRLIEKQAPENKAFVL
SSVDELEQQRDEIVSYLCDLAPEAPPPTLPPDMAQVTVGPGLLGV
STLGPKRNSMVLDVAFVLEGSDKIGEADENRSKEFMEEVIQRMDV
GQDSIHVTVLQYSYMVTVEYPFSEAQSKGDILQRVREIRYQGGNR
TNTGLALRYLSDHSELVSQGDREQAPNLVYMVTGNPASDEIKRLP
GDIQVVPIGVGPNANVQELERIGWPNAPILIQDFETLPREAPDLV
LQRCCSGEGLQIPTLSPAPDCSQPLDVILLLDGSSSFPASYFDEM
KSFAKAFISKANIGPRLTQVSVLQYGSITTIDVPWNVVPEKAHLL
SLVDVMQREGGPSQIGDALGFAVRYLTSEMHGARPGASKAVVILV
TDVSVDSVDAAADAARSNRVTVFPIGIGDRYDAAQLRILAGPAGD
SNVVKLQRIEDLPTMVTLGNSFLHKLCSGFVRICMDEDGNEKRPG
DVWTLPDQCHTVTCQPDGQTLLKSHRVNCDRGLRPSCPNSQSPVK
VEETCGCRWTCPCVCTGSSTRHIVTEDGQNFKLTGSCSYVLFQNK
EQDLEVILHNGACSPGARQGCMKSIEVKHSALSVELHSDMEVTVN
GRLVSVPYVGGNMEVNVYGAIMHEVRENHIGHIFTFTPQNNEFQL
QLSPKTFASKTYGLCGICDENGANDFMLRDGTVTTDWKTLVQEWT
VQRPGQTCQPILEEQCLVPDSSHCQVLLLPLFAECHKVLAPATFY
AICQQDSCHQEQVCEVIASYAHLCRTNGVCVDWRTPDFCAMSCPP
SLVYNHCEHGCPRHCDGNVSSCGDHPSEGCFCPPDKVMLEGSCVP
EEACTQCIGEDGVQHQFLEAWVPDHQPCQICTCLSGRKVNCTTQP
CPTAKAPTCGLCEVARLRQNADQCCPEYECVCDPVSCDLPPVPHC
ERGLQPTLINPGECRPNFTCACRKEECKRVSPPSCPPHRLPTLRK
TQCCDEYECACNCVNSTVSCPLGYLASTATNDCGCTTTTCLPDKV
CVHRSTIYPVGQFWEEGCDVCTCTDMEDAVMGLRVAQCSQKPCED
SCRSGFTYVLHEGECCGRCLPSACEVVTGSPRGDSQSSWKSVGSQ
WASPENPCLINECVRVKEEVFIQQRNVSCPQLEVPVCPSGFQLSC
KTSACCPSCRCERMEACMLNGTVIGPGKTVMIDVCTTCRCMVQVG
VISGFKLECRKTTCNPCPLGYKEENNTGECCGRCLPTACTIQLRG
GQIMTLKRDETLQDGCDTHFCKVNERGEYFWEKRVTGCPPFDEHK
CLAEGGKIMKIPGTCCDTCEEPECNDITARLQYVKVGSCKSEVEV
DIHYCQGKCASKAMYSIDINDVQDQCSCCSPTRTEPMQVALHCIN
GSVVYHEVLNAMECKCSPRKCSK

The antibody or antigen-binding fragment thereof, may therefore bind to a region between amino acid positions 2255 and 2722 of SEQ ID No: 1, which correspond to the C1-C6 domains of VWF.

Thus, preferably, the antibody or antigen-binding fragment thereof may bind to one or more amino acids between amino acid positions 2255 and 2722 of VWF, corresponding to the C1-C6 domains, which is provided herein as SEQ ID No: 2, as follows:

[SEQ ID No: 2]
TQCIGEDGVQHQFLEAWVPDHQPCQICTCLSGRKVNCTTQPCPTA
KAPTCGLCEVARLRQNADQCCPEYECVCDPVSCDLPPVPHCERGL
QPTLTNPGECRPNFTCACRKEECKRVSPPSCPPHRLPTLRKTQCC
DEYECACNCVNSTVSCPLGYLASTATNDCGCTTTTCLPDKVCVHR
STIYPVGQFWEEGCDVCTCTDMEDAVMGLRVAQCSQKPCEDSCRS
GFTYVLHEGECCGRCLPSACEVVTGSPRGDSQSSWKSVGSQWASP
ENPCLINECVRVKEEVFIQQRNVSCPQLEVPVCPSGFQLSCKTSA
CCPSCRCERMEACMLNGTVIGPGKTVMIDVCTTCRCMVQVGVISG
FKLECRKTTCNPCPLGYKEENNTGECCGRCLPTACTIQLRGGQIM
TLKRDETLQDGCDTHFCKVNERGEYFWEKRVTGCPPFDEHKCLAE
GGKIMKIPGTCCDTCEEP

Thus, preferably the antibody or antigen-binding fragment thereof binds to one or more amino acids within a sequence comprising or consisting of a sequence as substantially set out in SEQ ID No: 2, or a variant or fragment thereof.

In an embodiment, the amino acid sequence of the C1 domain of VWF may be provided herein as SEQ ID No: 3, as follows:

[SEQ ID No: 3]
TQCIGEDGVQHQFLEAWVPDHQPCQICTCLSGRKVNCTTQPCPTA
KAPTCGLCEVARLRQNADQCCPEYECVCDPVSCD

Thus, preferably the antibody or antigen-binding fragment thereof binds to one or more amino acids or an epitope within a sequence comprising or consisting of a sequence as substantially set out in SEQ ID No: 3, or a variant or fragment thereof.

In an embodiment, the amino acid sequence of the C2 domain of VWF may be provided herein as SEQ ID No: 4, as follows:

[SEQ ID No: 4]
LPPVPHCERGLQPTLTNPGECRPNFTCACRKEECKRVSPPSCPPH
RLPTLRKTQCCDEYECACNCVNST

Thus, preferably the antibody or antigen-binding fragment thereof binds to one or more amino acids or an epitope within a sequence comprising or consisting of a sequence as substantially set out in SEQ ID No: 4, or a variant or fragment thereof.

In an embodiment, the amino acid sequence of the C3 domain of VWF may be provided herein as SEQ ID No: 5, as follows:

[SEQ ID No: 5]
VCVHRSTIYPVGQFWEEGCDVCTCTDMEDAVMGLRVAQCSQKPCE
DSCRSGFTYVLHEGECCGRCLP

Thus, preferably the antibody or antigen-binding fragment thereof binds to one or more amino acids or an epitope within a sequence comprising or consisting of a sequence as substantially set out in SEQ ID No: 5, or a variant or fragment thereof.

In an embodiment, the amino acid sequence of the C4 domain of VWF may be provided herein as SEQ ID No: 6, as follows:

[SEQ ID No: 6]
SACEVVTGSPRGDSQSSWKSVGSQWASPENPCLINECVRVKEEVF
IQQRNVSCPQLEVPVCPSGFQLSCKTSACCPSCRCE

Thus, preferably the antibody or antigen-binding fragment thereof binds to one or more amino acids or an epitope within a sequence comprising or consisting of a sequence as substantially set out in SEQ ID No: 6, or a variant or fragment thereof.

In an embodiment, the amino acid sequence of the C5 domain of VWF may be provided herein as SEQ ID No: 7, as follows:

[SEQ ID No: 7]
RMEACMLNGTVIGPGKTVMIDVCTTCRCMVQVGVISGFKLECRKT
TCNPCPLGYKEENNTGECCGRCLP

Thus, preferably the antibody or antigen-binding fragment thereof binds to one or more amino acids or an epitope within a sequence comprising or consisting of a sequence as substantially set out in SEQ ID No: 7, or a variant or fragment thereof.

In an embodiment, the amino acid sequence of the C6 domain of VWF may be provided herein as SEQ ID No: 8, as follows:

[SEQ ID No: 8]
TACTIQLRGGQIMTLKRDETLQDGCDTHFCKVNERGEYFWEKRVT
GCPPFDEHKCLAEGGKIMKIPGTCCDTCEEP

Thus, preferably the antibody or antigen-binding fragment thereof binds to one or more amino acids or an epitope within a sequence comprising or consisting of a sequence as substantially set out in SEQ ID No: 8, or a variant or fragment thereof.

Previous therapeutic targeting of VWF has focused on the A1 and A3 domains (see FIG. 1), for example Caplacizumab which targets A1, and 82D6A3 which targets A3. However, the inventors have appreciated that the A1 and A3 domains are essential for platelet binding. As such, targeting either of the A1 and A3 domains, inhibits platelet binding under conditions of low shear rate, i.e. normal conditions, resulting in a severe bleeding risk in patients, which should be avoided.

Therefore, it is important that the antibody or antigen-binding fragment thereof of the invention, which targets one or more of the C1, C2, C3, C4, C5, and/or C6 domains of VWF does so specifically, and has no or little cross-reactivity with the A1, A2, and/or A3 domains of VWF, because this could result in significant unwanted off-target effects, such as a severe risk of bleeding.

Accordingly, preferably the antibody or antigen-binding fragment thereof of the invention does not substantially bind to an A1, A2, and/or A3 domain of VWF. Preferably, the antibody or antigen-binding fragment thereof of the invention has substantially no cross-reactivity with an A1, A2, and/or A3 domain of VWF. Most preferably, the antibody or antigen-binding fragment thereof of the invention has substantially no cross-reactivity with the A1 domain of VWF.

In an embodiment, the amino acid sequence of the A1 domain of VWF may be provided herein as SEQ ID No: 9, as follows:

[SEQ ID No: 9]
DLVFLLDGSSRLSEAEFEVLKAFVVDMMERLRISQKWVRVAVVEY
HDGSHAYIGLKDRKRPSELRRIASQVKYAGSQVASTSEVLKYTLF
QIFSKIDRPEASRITLLLMASQEPQRMSRNFVRYVQGLKKKKVIV
IPVGIGPHANLKQIRLIEKQAPENKAFVLSSVDELEQQRDEI

Thus, preferably the antibody or antigen-binding fragment thereof does not bind to a sequence as substantially set out in SEQ ID No: 9, or a variant or fragment thereof.

In an embodiment, the amino acid sequence of the A2 domain of VWF may be provided herein as SEQ ID No: 10, as follows:

[SEQ ID No: 10]
DVAFVLEGSDKIGEADFNRSKEFMEEVIQRMDVGQDSIHVTVLQY
SYMVTVEYPFSEAQSKGDILQRVREIRYQGGNRTNTGLALRYLSD
HSFLVSQGDREQAPNLVYMVTGNPASDEIKRLPGDIQVVPIGVGP
NANVQELERIGWPNAPILIQDFETLPREAPDLVQRCC

Thus, preferably the antibody or antigen-binding fragment thereof does not bind to a sequence as substantially set out in SEQ ID No: 10, or a variant or fragment thereof.

In an embodiment, the amino acid sequence of the A3 domain of VWF may be provided herein as SEQ ID No: 11, as follows:

[SEQ ID No: 11]
DVILLLDGSSSFPASYFDEMKSFAKAFISKANIGPRLTQVSVLQY
GSITTIDVPWNVVPEKAHLLSLVDVMQREGGPSQIGDALGFAVRY
LTSEMHGARPGASKAVVILVTDVSVDSVDAAADAARSNRVTVFPI
GIGDRYDAAQLRILAGPAGDSNVVKLQRIEDLPTMVTLGNSFLHK
L

Thus, preferably the antibody or antigen-binding fragment thereof does not bind to a sequence as substantially set out in SEQ ID No: 11, or a variant or fragment thereof.

The invention extends to both whole antibodies (i.e. immunoglobulins) with immunospecificity for one or more of the C1, C2, C3, C4, C5, and/or C6 domain of VWF (preferably C5), as well as to antigen-binding fragments or regions of the corresponding full-length antibody.

The antibody or antigen-binding fragment thereof may be monovalent, divalent or polyvalent. Monovalent antibodies are dimers (HL) comprising a heavy (H) chain associated by a disulphide bridge with a light chain (L). Divalent antibodies are tetramer (H2L2) comprising two dimers associated by at least one disulphide bridge. Polyvalent antibodies may also be produced, for example by linking multiple dimers. The basic structure of an antibody molecule consists of two identical light chains and two identical heavy chains which associate non-covalently and can be linked by disulphide bonds. Each heavy and light chain contains an amino-terminal variable region of about 110 amino acids, and constant sequences in the remainder of the chain. The variable region includes several hypervariable regions, or Complementarity Determining Regions (CDRs), that form the antigen-binding site of the antibody molecule and determine its specificity for the antigen, i.e. one or more of a C1, C2, C3, C4, C5, and/or C6 domain of VWF (preferably C5), or variant or fragment thereof (e.g. an epitope). On either side of the CDRs of the heavy and light chains is a framework region, a relatively conserved sequence of amino acids that anchors and orients the CDRs. Antibody fragments may include a bi-specific antibody (BsAb) or a chimeric antigen receptor (CAR).

The heavy chain constant region typically comprises three domains, C_{H1}, C_{H2}, and C_{H3}. Each light chain typically comprises a light chain variable region (V_{L}) and a light chain constant region. The light chain constant region typically comprises one domain, abbreviated CL.

Each heavy chain and light chain generally comprise three CDRs and four FRs, arranged in the following order (from N-terminus to C-terminus): FR1-CDR1-FR2-CDR2-FR3-CDR3-FR4. The CDRs are involved in antigen binding and confer antigen specificity and binding affinity to the antibody. See Kabat et al., Sequences of Proteins of Immunological Interest 5th ed. (1991) Public Health Service, National Institutes of Health, Bethesda, MD, incorporated by reference in its entirety.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2. The IgG antibody class is preferred.

The light chain from any vertebrate species can be assigned to one of two types, called kappa and lambda, based on the sequence of the constant domain.

The constant region consists of one of five heavy chain sequences (μ, γ, ζ, α, or ε) and one of two light chain sequences (κ or λ). The heavy chain constant region sequences determine the isotype of the antibody and the effector functions of the molecule.

Preferably, the antibody or antigen-binding fragment thereof is isolated or purified.

In one preferred embodiment, the antibody or antigen-binding fragment thereof comprises a polyclonal antibody, or an antigen-binding fragment thereof. The antibody or antigen-binding fragment thereof may be generated in a rabbit, mouse or rat.

Preferably, the antibody or antigen-binding fragment thereof is obtained by immunising a host animal with a C1-C6-Fc protein, or a variant or fragment thereof, such as any one or more of C1, C2, C3, C4, C5, and/or C6 domain, and then collecting the antibody or antigen-binding fragment thereof. The host animal may be a rabbit. Preferably, the host animal is a mouse.

In another preferred embodiment, the antibody or antigen-binding fragment thereof comprises a monoclonal antibody or an antigen-binding fragment thereof. The antibody or fragment thereof of may be mammalian. Preferably, the antibody of the invention is a human antibody. As used herein, the term “human antibody” can mean an antibody, such as a monoclonal antibody, which comprises substantially the same heavy and light chain CDR amino acid sequences as found in a particular human antibody exhibiting immunospecificity for one or more of C1, C2, C3, C4, C5, and/or C6 domains of VWF (preferably C5), or a variant or fragment thereof. An amino acid sequence, which is substantially the same as a heavy or light chain CDR, exhibits a considerable amount of sequence identity when compared to a reference sequence. Such identity is definitively known or recognisable as representing the amino acid sequence of the particular human antibody. Substantially the same heavy and light chain CDR amino acid sequence can have, for example, minor modifications or conservative substitutions of amino acids. Such a human antibody or fragment thereof maintains its function of selectively binding to at least one of the C1, C2, C3, C4, C5, and/or C6 domains of VWF (preferably C5), or a variant or fragment thereof.

The term “human monoclonal antibody” can include a monoclonal antibody with substantially or entirely human CDR amino acid sequences produced, for example by recombinant methods, such as production by a phage library, by lymphocytes or by hybridoma cells.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (by so-called “affinity maturation”), to humanize the antibody, to improve its production in cell culture, and/or to reduce its immunogenicity in a subject.

The term “humanised antibody” can mean an antibody from a non-human species (e.g. mouse or rabbit) whose protein sequences have been modified to increase their similarity to antibodies produced naturally in humans.

The antibody may be a recombinant antibody. The term “recombinant human antibody” can include a human antibody produced using recombinant DNA technology.

The term “antigen-binding fragment” can mean a region of the antibody having specific binding affinity for its target antigen, for example, one or more of a C1, C2, C3, C4, C5, and/or C6 domain of VWF (preferably C5), or a variant or fragment thereof. Preferably, the fragment is an epitope. The epitope may be linear or conformational. The antigen-binding region may be a hypervariable CDR or a functional portion thereof. The term “functional portion” of a CDR can mean a sequence within the CDR which shows specific affinity for the target antigen. The functional portion of a CDR may comprise a ligand which specifically binds to one or more of a C1, C2, C3, C4, C5, and/or C6 domain of VWF (preferably C5), or a fragment thereof.

The term “CDR” can mean a hypervariable region in the heavy and light variable chains. There may be one, two, three or more CDRs in each of the heavy and light chains of the antibody. Normally, there are at least three CDRs on each chain which, when configured together, form the antigen-binding site, i.e. the three-dimensional combining site with which the antigen binds or specifically reacts. It has however been postulated that there may be four CDRs in the heavy chains of some antibodies.

The definition of CDR also includes overlapping or subsets of amino acid residues when compared against each other. The exact residue numbers which encompass a particular CDR or a functional portion thereof will vary depending on the sequence and size of the CDR. Those skilled in the art can routinely determine which residues comprise a particular CDR given the variable region amino acid sequence of the antibody.

The amino acid sequence boundaries of a CDR can be determined by using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme).

The term “functional fragment” of an antibody can mean a portion of the antibody which retains a functional activity. A functional activity can be, for example antigen binding activity or specificity. A functional activity can also be, for example, an effector function provided by an antibody constant region. The term “functional fragment” is also intended to include, for example, fragments produced by protease digestion or reduction of a human monoclonal antibody and by recombinant DNA methods known to those skilled in the art. Human monoclonal antibody functional fragments include, for example individual heavy or light chains and fragments thereof, such as VL, VH and Fd; monovalent fragments, such as Fv, Fab, and Fab′; bivalent fragments such as F(ab′)₂; single chain Fv (scFv); and Fc fragments. Alternatively, as discussed hereinafter, and as exemplified, the Fc fragment of the antibody may be disabled by introducing amino acid substitutions into the Fc region, which silence or reduce the effector function of the antibody.

The term “VL fragment” can mean a fragment of the light chain of a human monoclonal antibody which includes all or part of the light chain variable region, including the CDRs. A VL fragment can further include light chain constant region sequences.

The term “VH fragment” can mean a fragment of the heavy chain of a human monoclonal antibody which includes all or part of the heavy chain variable region, including the CDRs.

The term “Fd fragment” can mean the heavy chain variable region coupled to the first heavy chain constant region, i.e. VH and CH-1. The “Fd fragment” does not include the light chain, or the second and third constant regions of the heavy chain.

The term “Fv fragment” can mean a monovalent antigen-binding fragment of a human monoclonal antibody, including all or part of the variable regions of the heavy and light chains, and absent of the constant regions of the heavy and light chains. The variable regions of the heavy and light chains include, for example, the CDRs. For example, an Fv fragment includes all or part of the amino terminal variable region of about 110 amino acids of both the heavy and light chains.

The term “Fab fragment” can mean a monovalent antigen-binding fragment of a human monoclonal antibody that is larger than an Fv fragment. For example, a Fab fragment includes the variable regions, and all or part of the first constant domain of the heavy and light chains. Thus, a Fab fragment additionally includes, for example, amino acid residues from about 110 to about 220 of the heavy and light chains.

The term “Fab′ fragment” can mean a monovalent antigen-binding fragment of a human monoclonal antibody that is larger than a Fab fragment. For example, a Fab′ fragment includes all of the light chain, all of the variable region of the heavy chain, and all or part of the first and second constant domains of the heavy chain. For example, a Fab′ fragment can additionally include some or all of amino acid residues 220 to 330 of the heavy chain.

The term “F(ab′), fragment” can mean a bivalent antigen-binding fragment of a human monoclonal antibody. An F(ab′)₂ fragment includes, for example, all or part of the variable regions of two heavy chains- and two light chains, and can further include all or part of the first constant domains of two heavy chains and two light chains.

The term “single chain Fv (scFv)” can mean a fusion of the variable regions of the heavy (VH) and light chains (VL) connected with a short linker peptide.

The term “bispecific antibody (BsAb)” can mean a bispecific antibody comprising two scFv linked to each other by a shorter linked peptide.

In one embodiment, the antigen-binding fragment thereof may be a single domain antibody (sdAb) (also referred to as a nanobody). The skilled person would understand that an sdAb is an antibody fragment consisting of a single monomeric variable antibody domain (referred to as a VHH).

Alternatively, in another embodiment, the antigen-binding fragment thereof is a single-chain antibody, an intrabody, a peptide (e.g. a bicyclic peptide), or any other type of fragment or protein scaffold.

One skilled in the art knows that the exact boundaries of a fragment of an antibody are not important, so long as the fragment maintains a functional activity. Using well-known recombinant methods, one skilled in the art can engineer a polynucleotide sequence to express a functional fragment with any endpoints desired for a particular application. A functional fragment of the antibody may comprise or consist of a fragment with substantially the same heavy and light chain variable regions as the human antibody.

Preferably, the antibody or antigen-binding fragment thereof, with respect to the first aspect of the invention, is immunospecific for an epitope within one or more of a C1, C2, C3, C4, C5, and/or C6 domain of VWF. In one preferred embodiment, the antibody or antigen-binding fragment thereof, is immunospecific for an epitope within the C1 domain of VWF. Alternatively, in another preferred embodiment, the antibody or antigen-binding fragment thereof, is immunospecific for an epitope within the C2 domain of VWF. Alternatively, in another preferred embodiment, the antibody or antigen-binding fragment thereof, is immunospecific for an epitope within the C3 domain of VWF. Alternatively, in another preferred embodiment, the antibody or antigen-binding fragment thereof, is immunospecific for an epitope within the C4 domain of VWF. Alternatively, in another preferred embodiment, the antibody or antigen-binding fragment thereof, is immunospecific for an epitope within the C5 domain of VWF. Alternatively, in another preferred embodiment, the antibody or antigen-binding fragment thereof, is immunospecific for an epitope within the C6 domain of VWF. The antigen-binding fragment thereof may comprise or consist of any of the fragments selected from a group consisting of VH, VL, Fd, Fv, Fab, Fab′, scFv, F (ab′)₂ and Fc fragment.

The antigen-binding fragment thereof may be a single domain antibody (sdAb), otherwise referred to as a nanobody, which the skilled person would understand is an antibody fragment consisting of a single monomeric variable antibody domain.

The antigen-binding fragment thereof may comprise or consist of any one of the antigen binding region sequences of the VL, any one of the antigen binding region sequences of the VH, or a combination of VL and VH antigen binding regions of a human antibody. The appropriate number and combination of VH and VL antigen binding region sequences may be determined by those skilled in the art depending on the desired affinity and specificity and the intended use of the antigen-binding fragment. Functional fragments or antigen-binding fragments of antibodies may be readily produced and isolated using methods well known to those skilled in the art. Such methods include, for example, proteolytic methods, recombinant methods and chemical synthesis. Proteolytic methods for the isolation of functional fragments comprise using human antibodies as a starting material. Enzymes suitable for proteolysis of human immunoglobulins may include, for example, papain, and pepsin. The appropriate enzyme may be readily chosen by one skilled in the art, depending on, for example, whether monovalent or bivalent fragments are required. For example, papain cleavage results in two monovalent Fab′ fragments that bind antigen and an Fc fragment. Pepsin cleavage, for example, results in a bivalent F(ab′) fragment. An F (ab′)₂ fragment of the invention may be further reduced using, for example, DTT or 2-mercaptoethanol to produce two monovalent Fab′ fragments.

Functional or antigen-binding fragments of antibodies produced by proteolysis may be purified by affinity and column chromatographic procedures. For example, undigested antibodies and Fc fragments may be removed by binding to protein A. Additionally, functional fragments may be purified by virtue of their charge and size, using, for example, ion exchange and gel filtration chromatography. Such methods are well known to those skilled in the art.

The antibody or antigen-binding fragment thereof may be produced by recombinant methodology. Preferably, one initially isolates a polynucleotide encoding desired regions of the antibody heavy and light chains. Such regions may include, for example, all or part of the variable region of the heavy and light chains. Preferably, such regions can particularly include the antigen binding regions of the heavy and light chains, preferably the antigen binding sites, most preferably the CDRs.

The polynucleotide encoding the antibody or antigen-binding fragment thereof according to the invention may be produced using methods known to those skilled in the art. The polynucleotide encoding the antibody or antigen-binding fragment thereof may be directly synthesized by methods of oligonucleotide synthesis known in the art. Alternatively, smaller fragments may be synthesized and joined to form a larger functional fragment using recombinant methods known in the art.

As used herein, the term “immunospecificity” can mean the binding region of the antibody or antigen-binding fragment thereof is capable of immunoreacting with one or more of a C1, C2, C3, C4, C5, and/or C6 domain of VWF (preferably C5), or a variant or fragment thereof, by specifically binding therewith. The antibody or antigen-binding fragment thereof can preferably selectively interact with an antigen (one or more of a C1, C2, C3, C4, C5, and/or C6 domain of VWF-preferably C5) with an affinity constant of approximately 10⁻⁵ to 10⁻¹³ M⁻¹, preferably 10⁻⁶ to 10⁻⁹ M⁻¹, even more preferably, 10⁻¹⁰ to 10⁻¹² M⁻¹.

The antibody or antigen-binding fragment thereof preferably does not substantially bind to A1, A2, and/or A3 domains of VWF, such that the affinity constant is approximately more than 10⁻¹⁰ M⁻¹, 10⁻⁹ M⁻¹, 10⁻⁸ M⁻¹, 10⁻⁷ M⁻¹, or 10⁻⁶ M⁻¹, preferably more than 10⁻⁵ M⁻¹, 10⁻⁴M⁻¹ or 10⁻³M⁻¹ and even more preferably 10⁻²M⁻¹ 10⁻¹M⁻¹ or 10⁻²M⁻¹ and most preferably 10⁺¹M⁻¹, 10⁺²M⁻¹ or 10⁺³M⁻¹.

The term “immunoreact” can mean the binding region is capable of eliciting an immune response upon binding with one or more of a C1, C2, C3, C4, C5, and/or C6 domain of VWF, or an epitope thereof.

The term “epitope” can mean any region of an antigen with the ability to elicit, and combine with, a binding region of the antibody or antigen-binding fragment thereof.

The epitope may be linear. This can mean that the antibody interacts with a plurality of continuous amino acids of the antigen, and so the epitope can consist of these defined amino acids.

Alternatively, the epitope may be conformational, i.e. non-linear or discontinuous. This can mean that the antibody interacts with multiple, distinct segments from the primary amino acid sequence of the antigen.

Thus, the antibody or antigen-binding fragment thereof may comprise a heavy chain. The heavy chain may be selected from the group consisting of IgA; IgD; IgE; IgG and IgM. Preferably, the heavy chain is an IgG. Preferably, the heavy chain is an IgA.

The heavy chain may be an IgG1. The heavy chain may be an IgG2. The heavy chain may be an IgG3. The heavy chain may be an IgG4. The heavy chain may be an IgA1. The heavy chain may be an IgA2.

As described in the Examples and as shown in FIGS. 4 and 5, the inventors have surprisingly demonstrated that the antibodies and antigen-binding fragments referred to herein as 25B05, 28A12, 32H07, 33D03, 4D09 13C11 and 16A11 8F04, are able to significantly target one or more of the C1-C6 domains of VWF, and each of these antibodies are defined below in detail. The CDR, VH, VL, HC and LC sequences of these six antibodies are conveniently summarised in the table shown in FIG. 6.

### 33D03

In one embodiment, the antibody or antigen-binding fragment thereof is referred to herein as 33D03.

Preferably, the antibody or antigen-binding fragment thereof comprises a CDR-H1 domain comprising or consisting of a sequence as substantially set out in SEQ ID No: 42, or a variant or fragment thereof. Preferably, the antibody or antigen-binding fragment thereof comprises a CDR-H2 domain comprising or consisting of a sequence as substantially set out in SEQ ID No: 43, or a variant or fragment thereof. Preferably, the antibody or antigen-binding fragment thereof comprises a CDR-H3 domain comprising or consisting of a sequence as substantially set out in SEQ ID No: 44, or a variant or fragment thereof.

Preferably, the antibody or antigen-binding fragment thereof comprises a CDR-H1 domain comprising or consisting of SEQ ID No: 42, a CDR-H2 domain comprising or consisting of SEQ ID No: 43 and/or a CDR-H3 domain comprising or consisting of SEQ ID No: 44. Preferably, however, the antibody or antigen-binding fragment thereof comprises a CDR-H1 domain comprising or consisting of SEQ ID No: 42, a CDR-H2 domain comprising or consisting of SEQ ID No: 43 and a CDR-H3 domain comprising or consisting of SEQ ID No: 44.

Preferably, the antibody or antigen-binding fragment thereof comprises a heavy chain variable (VH) region comprising or consisting of a sequence as substantially set out in SEQ ID No: 45, or a variant or fragment thereof.

Preferably, the antibody or antigen-binding fragment thereof comprises a heavy chain variable (VH) region encoded by a nucleic acid sequence as substantially set out in SEQ ID No: 78, or a variant or fragment thereof.

Preferably, the antibody or antigen-binding fragment thereof comprises a CDR-L1 domain comprising or consisting of a sequence as substantially set out in SEQ ID No: 46, or a variant or fragment thereof. Preferably, the antibody or antigen-binding fragment thereof comprises a CDR-L2 domain comprising or consisting of a sequence as substantially set out in SEQ ID No: 47, or a variant or fragment thereof. Preferably, the antibody or antigen-binding fragment thereof comprises a CDR-L3 domain comprising or consisting of a sequence as substantially set out in SEQ ID No: 48, or a variant or fragment thereof.

Preferably, the antibody or antigen-binding fragment thereof comprises a CDR-L1 domain comprising or consisting of SEQ ID No: 46, a CDR-L2 domain comprising or consisting of SEQ ID No: 47, and/or a CDR-L3 domain comprising or consisting of SEQ ID No: 48. However, preferably the antibody or antigen-binding fragment thereof comprises a CDR-L1 domain comprising or consisting of SEQ ID No: 46, a CDR-L2 domain comprising or consisting of SEQ ID No: 47, and a CDR-L3 domain comprising or consisting of SEQ ID No: 48.

Preferably, the antibody or antigen-binding fragment thereof comprises a light chain variable region comprising or consisting of a sequence as substantially set out in SEQ ID No: 49, or a variant or fragment thereof.

Preferably, the antibody or antigen-binding fragment thereof comprises a light chain variable (VL) region encoded by a nucleic acid sequence as substantially set out in SEQ ID No: 79, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 42, a CDR-H2 domain comprising or consisting of SEQ ID No: 43; a CDR-H3 domain comprising or consisting of SEQ ID No: 44, a CDR-L1 domain comprising or consisting of SEQ ID No: 46, a CDR-L2 domain comprising or consisting of SEQ ID No: 47, and a CDR-L3 domain comprising or consisting of SEQ ID No: 48.

Preferably, the antibody or antigen-binding fragment thereof comprises a heavy chain variable region comprising or consisting of SEQ ID No: 45, and a light chain variable region comprising or consisting of SEQ ID No: 49.

Preferably, the antibody or antigen-binding fragment thereof comprises a heavy chain variable region encoded by a nucleic acid sequence comprising or consisting of SEQ ID No: 78, and a light chain variable region encoded by a nucleic acid sequence comprising or consisting of SEQ ID No: 79.

Preferably, the antibody or antigen-binding fragment thereof comprises a heavy chain constant (HC) region comprising or consisting of a sequence as substantially set out in SEQ ID No: 50, or a variant or fragment thereof.

Preferably, the antibody or antigen-binding fragment thereof comprises a light chain constant (LC) region comprising or consisting of a sequence as substantially set out in SEQ ID No: 51, or a variant or fragment thereof.

Preferably, the antibody or antigen-binding fragment thereof comprises a heavy chain constant region comprising or consisting of SEQ ID No: 50 and a light chain constant region comprising or consisting of SEQ ID No: 51.

### 25B05 H1

Accordingly, in one embodiment, the antibody or antigen-binding fragment thereof is referred to herein as 25B05_H1.

Preferably, the antibody or antigen-binding fragment thereof comprises a CDR-H1 domain comprising or consisting of a sequence as substantially set out in SEQ ID No: 12, or a variant or fragment thereof. Preferably, the antibody or antigen-binding fragment thereof comprises a CDR-H2 domain comprising or consisting of a sequence as substantially set out in SEQ ID No: 13, or a variant or fragment thereof. Preferably, the antibody or antigen-binding fragment thereof comprises a CDR-H3 domain comprising or consisting of a sequence as substantially set out in SEQ ID No: 14, or a variant or fragment thereof.

Preferably, the antibody or antigen-binding fragment thereof comprises a CDR-H1 domain comprising or consisting of SEQ ID No: 12, a CDR-H2 domain comprising or consisting of SEQ ID No: 13 and/or a CDR-H3 domain comprising or consisting of SEQ ID No: 14. Preferably, however, the antibody or antigen-binding fragment thereof comprises a CDR-H1 domain comprising or consisting of SEQ ID No: 12, a CDR-H2 domain comprising or consisting of SEQ ID No: 13 and a CDR-H3 domain comprising or consisting of SEQ ID No: 14.

Preferably, the antibody or antigen-binding fragment thereof comprises a heavy chain variable (VH) region comprising or consisting of a sequence as substantially set out in SEQ ID No: 15, or a variant or fragment thereof.

Preferably, the antibody or antigen-binding fragment thereof comprises a heavy chain variable (VH) region encoded by a nucleic acid sequence as substantially set out in SEQ ID No: 72, or a variant or fragment thereof.

Preferably, the antibody or antigen-binding fragment thereof comprises a CDR-L1 domain comprising or consisting of a sequence as substantially set out in SEQ ID No: 16, or a variant or fragment thereof. Preferably, the antibody or antigen-binding fragment thereof comprises a CDR-L2 domain comprising or consisting of a sequence as substantially set out in SEQ ID No: 17, or a variant or fragment thereof. Preferably, the antibody or antigen-binding fragment thereof comprises a CDR-L3 domain comprising or consisting of a sequence as substantially set out in SEQ ID No: 18, or a variant or fragment thereof.

Preferably, the antibody or antigen-binding fragment thereof comprises a CDR-L1 domain comprising or consisting of SEQ ID No: 16, a CDR-L2 domain comprising or consisting of SEQ ID No: 17, and/or a CDR-L3 domain comprising or consisting of SEQ ID No: 18. However, preferably the antibody or antigen-binding fragment thereof comprises a CDR-L1 domain comprising or consisting of SEQ ID No: 16, a CDR-L2 domain comprising or consisting of SEQ ID No: 17, and a CDR-L3 domain comprising or consisting of SEQ ID No: 18.

Preferably, the antibody or antigen-binding fragment thereof comprises a light chain variable region comprising or consisting of a sequence as substantially set out in SEQ ID No: 19, or a variant or fragment thereof.

Preferably, the antibody or antigen-binding fragment thereof comprises a light chain variable (VL) region encoded by a nucleic acid sequence as substantially set out in SEQ ID No: 73, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 12, a CDR-H2 domain comprising or consisting of SEQ ID No: 13; a CDR-H3 domain comprising or consisting of SEQ ID No: 14, a CDR-L1 domain comprising or consisting of SEQ ID No: 16, a CDR-L2 domain comprising or consisting of SEQ ID No: 17, and a CDR-L3 domain comprising or consisting of SEQ ID No: 18.

Preferably, the antibody or antigen-binding fragment thereof comprises a heavy chain variable region comprising or consisting of SEQ ID No: 15, and a light chain variable region comprising or consisting of SEQ ID No: 19.

Preferably, the antibody or antigen-binding fragment thereof comprises a heavy chain variable region encoded by a nucleic acid sequence comprising or consisting of SEQ ID No: 72, and a light chain variable region encoded by a nucleic acid sequence comprising or consisting of SEQ ID No: 73.

Preferably, the antibody or antigen-binding fragment thereof comprises a heavy chain constant (HC) region comprising or consisting of a sequence as substantially set out in SEQ ID No: 20, or a variant or fragment thereof.

Preferably, the antibody or antigen-binding fragment thereof comprises a light chain constant (LC) region comprising or consisting of a sequence as substantially set out in SEQ ID No: 21, or a variant or fragment thereof.

Preferably, the antibody or antigen-binding fragment thereof comprises a heavy chain constant region comprising or consisting of SEQ ID No: 20 and a light chain constant region comprising or consisting of SEQ ID No: 21.

### 28A12

In one embodiment, the antibody or antigen-binding fragment thereof is referred to herein as 28A12.

Preferably, the antibody or antigen-binding fragment thereof comprises a CDR-H1 domain comprising or consisting of a sequence as substantially set out in SEQ ID No: 22, or a variant or fragment thereof. Preferably, the antibody or antigen-binding fragment thereof comprises a CDR-H2 domain comprising or consisting of a sequence as substantially set out in SEQ ID No: 23, or a variant or fragment thereof. Preferably, the antibody or antigen-binding fragment thereof comprises a CDR-H3 domain comprising or consisting of a sequence as substantially set out in SEQ ID No: 24, or a variant or fragment thereof.

Preferably, the antibody or antigen-binding fragment thereof comprises a CDR-H1 domain comprising or consisting of SEQ ID No: 22, a CDR-H2 domain comprising or consisting of SEQ ID No: 23 and/or a CDR-H3 domain comprising or consisting of SEQ ID No: 24. Preferably, however, the antibody or antigen-binding fragment thereof comprises a CDR-H1 domain comprising or consisting of SEQ ID No: 22, a CDR-H2 domain comprising or consisting of SEQ ID No: 23 and a CDR-H3 domain comprising or consisting of SEQ ID No: 24.

Preferably, the antibody or antigen-binding fragment thereof comprises a heavy chain variable (VH) region comprising or consisting of a sequence as substantially set out in SEQ ID No: 25, or a variant or fragment thereof.

Preferably, the antibody or antigen-binding fragment thereof comprises a heavy chain variable (VH) region encoded by a nucleic acid sequence as substantially set out in SEQ ID No: 74, or a variant or fragment thereof.

Preferably, the antibody or antigen-binding fragment thereof comprises a CDR-L1 domain comprising or consisting of a sequence as substantially set out in SEQ ID No: 26, or a variant or fragment thereof. Preferably, the antibody or antigen-binding fragment thereof comprises a CDR-L2 domain comprising or consisting of a sequence as substantially set out in SEQ ID No: 27, or a variant or fragment thereof. Preferably, the antibody or antigen-binding fragment thereof comprises a CDR-L3 domain comprising or consisting of a sequence as substantially set out in SEQ ID No: 28, or a variant or fragment thereof.

Preferably, the antibody or antigen-binding fragment thereof comprises a CDR-L1 domain comprising or consisting of SEQ ID No: 26, a CDR-L2 domain comprising or consisting of SEQ ID No: 27, and/or a CDR-L3 domain comprising or consisting of SEQ ID No: 28. However, preferably the antibody or antigen-binding fragment thereof comprises a CDR-L1 domain comprising or consisting of SEQ ID No: 26, a CDR-L2 domain comprising or consisting of SEQ ID No: 27, and a CDR-L3 domain comprising or consisting of SEQ ID No: 28.

Preferably, the antibody or antigen-binding fragment thereof comprises a light chain variable region comprising or consisting of a sequence as substantially set out in SEQ ID No: 29, or a variant or fragment thereof.

Preferably, the antibody or antigen-binding fragment thereof comprises a light chain variable (VL) region encoded by a nucleic acid sequence as substantially set out in SEQ ID No: 75, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 22, a CDR-H2 domain comprising or consisting of SEQ ID No: 23; a CDR-H3 domain comprising or consisting of SEQ ID No: 24, a CDR-L1 domain comprising or consisting of SEQ ID No: 26, a CDR-L2 domain comprising or consisting of SEQ ID No: 27, and a CDR-L3 domain comprising or consisting of SEQ ID No: 28.

Preferably, the antibody or antigen-binding fragment thereof comprises a heavy chain variable region comprising or consisting of SEQ ID No: 25, and a light chain variable region comprising or consisting of SEQ ID No: 29.

Preferably, the antibody or antigen-binding fragment thereof comprises a heavy chain variable region encoded by a nucleic acid sequence comprising or consisting of SEQ ID No: 74, and a light chain variable region encoded by a nucleic acid sequence comprising or consisting of SEQ ID No: 75.

Preferably, the antibody or antigen-binding fragment thereof comprises a heavy chain constant (HC) region comprising or consisting of a sequence as substantially set out in SEQ ID No: 30, or a variant or fragment thereof.

Preferably, the antibody or antigen-binding fragment thereof comprises a light chain constant (LC) region comprising or consisting of a sequence as substantially set out in SEQ ID No: 31, or a variant or fragment thereof.

Preferably, the antibody or antigen-binding fragment thereof comprises a heavy chain constant region comprising or consisting of SEQ ID No: 30 and a light chain constant region comprising or consisting of SEQ ID No: 31.

### 16A11 8F04

In one embodiment, the antibody or antigen-binding fragment thereof is referred to herein as 16A11 8F04.

Preferably, the antibody or antigen-binding fragment thereof comprises a CDR-H1 domain comprising or consisting of a sequence as substantially set out in SEQ ID No: 62, or a variant or fragment thereof. Preferably, the antibody or antigen-binding fragment thereof comprises a CDR-H2 domain comprising or consisting of a sequence as substantially set out in SEQ ID No: 63, or a variant or fragment thereof. Preferably, the antibody or antigen-binding fragment thereof comprises a CDR-H3 domain comprising or consisting of a sequence as substantially set out in SEQ ID No: 64, or a variant or fragment thereof.

Preferably, the antibody or antigen-binding fragment thereof comprises a CDR-H1 domain comprising or consisting of SEQ ID No: 62, a CDR-H2 domain comprising or consisting of SEQ ID No: 63 and/or a CDR-H3 domain comprising or consisting of SEQ ID No: 64. Preferably, however, the antibody or antigen-binding fragment thereof comprises a CDR-H1 domain comprising or consisting of SEQ ID No: 62, a CDR-H2 domain comprising or consisting of SEQ ID No: 63 and a CDR-H3 domain comprising or consisting of SEQ ID No: 64.

Preferably, the antibody or antigen-binding fragment thereof comprises a heavy chain variable (VH) region comprising or consisting of a sequence as substantially set out in SEQ ID No: 65, or a variant or fragment thereof.

Preferably, the antibody or antigen-binding fragment thereof comprises a heavy chain variable (VH) region encoded by a nucleic acid sequence as substantially set out in SEQ ID No: 82, or a variant or fragment thereof.

Preferably, the antibody or antigen-binding fragment thereof comprises a CDR-L1 domain comprising or consisting of a sequence as substantially set out in SEQ ID No: 66, or a variant or fragment thereof. Preferably, the antibody or antigen-binding fragment thereof comprises a CDR-L2 domain comprising or consisting of a sequence as substantially set out in SEQ ID No: 67, or a variant or fragment thereof. Preferably, the antibody or antigen-binding fragment thereof comprises a CDR-L3 domain comprising or consisting of a sequence as substantially set out in SEQ ID No: 68, or a variant or fragment thereof.

Preferably, the antibody or antigen-binding fragment thereof comprises a CDR-L1 domain comprising or consisting of SEQ ID No: 66, a CDR-L2 domain comprising or consisting of SEQ ID No: 67, and/or a CDR-L3 domain comprising or consisting of SEQ ID No: 68. However, preferably the antibody or antigen-binding fragment thereof comprises a CDR-L1 domain comprising or consisting of SEQ ID No: 66, a CDR-L2 domain comprising or consisting of SEQ ID No: 67, and a CDR-L3 domain comprising or consisting of SEQ ID No: 68.

Preferably, the antibody or antigen-binding fragment thereof comprises a light chain variable region comprising or consisting of a sequence as substantially set out in SEQ ID No: 69, or a variant or fragment thereof.

Preferably, the antibody or antigen-binding fragment thereof comprises a light chain variable (VL) region encoded by a nucleic acid sequence as substantially set out in SEQ ID No: 83, or a variant or fragment thereof.

Preferably, the antibody or antigen-binding fragment thereof comprises at least one, at antibody or antigen-binding fragment thereof comprises at least CDR-H3.

Preferably, the antibody or antigen-binding fragment thereof comprises a CDR-H1 domain comprising or consisting of SEQ ID No: 62, a CDR-H2 domain comprising or consisting of SEQ ID No: 63; a CDR-H3 domain comprising or consisting of SEQ ID No: 64, a CDR-L1 domain comprising or consisting of SEQ ID No: 66, a CDR-L2 domain comprising or consisting of SEQ ID No: 67, and a CDR-L3 domain comprising or consisting of SEQ ID No: 68.

Preferably, the antibody or antigen-binding fragment thereof comprises a heavy chain variable region comprising or consisting of SEQ ID No: 65, and a light chain variable region comprising or consisting of SEQ ID No: 69.

Preferably, the antibody or antigen-binding fragment thereof comprises a heavy chain variable region encoded by a nucleic acid sequence comprising or consisting of SEQ ID No: 82, and a light chain variable region encoded by a nucleic acid sequence comprising or consisting of SEQ ID No: 83.

Preferably, the antibody or antigen-binding fragment thereof comprises a heavy chain constant (HC) region comprising or consisting of a sequence as substantially set out in SEQ ID No: 70, or a variant or fragment thereof.

Preferably, the antibody or antigen-binding fragment thereof comprises a light chain constant (LC) region comprising or consisting of a sequence as substantially set out in SEQ ID No: 71, or a variant or fragment thereof.

Preferably, the antibody or antigen-binding fragment thereof comprises a heavy chain constant region comprising or consisting of SEQ ID No: 70 and a light chain constant region comprising or consisting of SEQ ID No: 71.

### 32H07

In one embodiment, the antibody or antigen-binding fragment thereof is referred to herein as 32H07.

Preferably, the antibody or antigen-binding fragment thereof comprises a CDR-H1 domain comprising or consisting of a sequence as substantially set out in SEQ ID No: 32, or a variant or fragment thereof. Preferably, the antibody or antigen-binding fragment thereof comprises a CDR-H2 domain comprising or consisting of a sequence as substantially set out in SEQ ID No: 33, or a variant or fragment thereof. Preferably, the antibody or antigen-binding fragment thereof comprises a CDR-H3 domain comprising or consisting of a sequence as substantially set out in SEQ ID No: 34, or a variant or fragment thereof.

Preferably, the antibody or antigen-binding fragment thereof comprises a CDR-H1 domain comprising or consisting of SEQ ID No: 32, a CDR-H2 domain comprising or consisting of SEQ ID No: 33 and/or a CDR-H3 domain comprising or consisting of SEQ ID No: 34. Preferably, however, the antibody or antigen-binding fragment thereof comprises a CDR-H1 domain comprising or consisting of SEQ ID No: 32, a CDR-H2 domain comprising or consisting of SEQ ID No: 33 and a CDR-H3 domain comprising or consisting of SEQ ID No: 34.

Preferably, the antibody or antigen-binding fragment thereof comprises a heavy chain variable (VH) region comprising or consisting of a sequence as substantially set out in SEQ ID No: 35, or a variant or fragment thereof.

Preferably, the antibody or antigen-binding fragment thereof comprises a heavy chain variable (VH) region encoded by a nucleic acid sequence as substantially set out in SEQ ID No: 76, or a variant or fragment thereof.

Preferably, the antibody or antigen-binding fragment thereof comprises a CDR-L1 domain comprising or consisting of a sequence as substantially set out in SEQ ID No: 36, or a variant or fragment thereof. Preferably, the antibody or antigen-binding fragment thereof comprises a CDR-L2 domain comprising or consisting of a sequence as substantially set out in SEQ ID No: 37, or a variant or fragment thereof. Preferably, the antibody or antigen-binding fragment thereof comprises a CDR-L3 domain comprising or consisting of a sequence as substantially set out in SEQ ID No: 38, or a variant or fragment thereof.

Preferably, the antibody or antigen-binding fragment thereof comprises a CDR-L1 domain comprising or consisting of SEQ ID No: 36, a CDR-L2 domain comprising or consisting of SEQ ID No: 37, and/or a CDR-L3 domain comprising or consisting of SEQ ID No: 38. However, preferably the antibody or antigen-binding fragment thereof comprises a CDR-L1 domain comprising or consisting of SEQ ID No: 36, a CDR-L2 domain comprising or consisting of SEQ ID No: 37, and a CDR-L3 domain comprising or consisting of SEQ ID No: 38.

Preferably, the antibody or antigen-binding fragment thereof comprises a light chain variable region comprising or consisting of a sequence as substantially set out in SEQ ID No: 39, or a variant or fragment thereof.

Preferably, the antibody or antigen-binding fragment thereof comprises a light chain variable (VL) region encoded by a nucleic acid sequence as substantially set out in SEQ ID No: 77, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 32, a CDR-H2 domain comprising or consisting of SEQ ID No: 33; a CDR-H3 domain comprising or consisting of SEQ ID No: 34, a CDR-L1 domain comprising or consisting of SEQ ID No: 36, a CDR-L2 domain comprising or consisting of SEQ ID No: 37, and a CDR-L3 domain comprising or consisting of SEQ ID No: 38.

Preferably, the antibody or antigen-binding fragment thereof comprises a heavy chain variable region comprising or consisting of SEQ ID No: 35, and a light chain variable region comprising or consisting of SEQ ID No: 39.

Preferably, the antibody or antigen-binding fragment thereof comprises a heavy chain variable region encoded by a nucleic acid sequence comprising or consisting of SEQ ID No: 76, and a light chain variable region encoded by a nucleic acid sequence comprising or consisting of SEQ ID No: 77.

Preferably, the antibody or antigen-binding fragment thereof comprises a heavy chain constant (HC) region comprising or consisting of a sequence as substantially set out in SEQ ID No: 40, or a variant or fragment thereof.

Preferably, the antibody or antigen-binding fragment thereof comprises a light chain constant (LC) region comprising or consisting of a sequence as substantially set out in SEQ ID No: 41, or a variant or fragment thereof.

Preferably, the antibody or antigen-binding fragment thereof comprises a heavy chain constant region comprising or consisting of SEQ ID No: 40 and a light chain constant region comprising or consisting of SEQ ID No: 41.

### 4D09 13C11

In one embodiment, the antibody or antigen-binding fragment thereof is referred to herein as 4D09 13C11.

Preferably, the antibody or antigen-binding fragment thereof comprises a CDR-H1 domain comprising or consisting of a sequence as substantially set out in SEQ ID No: 52, or a variant or fragment thereof. Preferably, the antibody or antigen-binding fragment thereof comprises a CDR-H2 domain comprising or consisting of a sequence as substantially set out in SEQ ID No: 53, or a variant or fragment thereof. Preferably, the antibody or antigen-binding fragment thereof comprises a CDR-H3 domain comprising or consisting of a sequence as substantially set out in SEQ ID No: 54, or a variant or fragment thereof.

Preferably, the antibody or antigen-binding fragment thereof comprises a CDR-H1 domain comprising or consisting of SEQ ID No: 52, a CDR-H2 domain comprising or consisting of SEQ ID No: 53 and/or a CDR-H3 domain comprising or consisting of SEQ ID No: 54. Preferably, however, the antibody or antigen-binding fragment thereof comprises a CDR-H1 domain comprising or consisting of SEQ ID No: 52, a CDR-H2 domain comprising or consisting of SEQ ID No: 53 and a CDR-H3 domain comprising or consisting of SEQ ID No: 54.

Preferably, the antibody or antigen-binding fragment thereof comprises a heavy chain variable (VH) region comprising or consisting of a sequence as substantially set out in SEQ ID No: 55, or a variant or fragment thereof.

Preferably, the antibody or antigen-binding fragment thereof comprises a heavy chain variable (VH) region encoded by a nucleic acid sequence as substantially set out in SEQ ID No: 80, or a variant or fragment thereof.

Preferably, the antibody or antigen-binding fragment thereof comprises a CDR-L1 domain comprising or consisting of a sequence as substantially set out in SEQ ID No: 56, or a variant or fragment thereof. Preferably, the antibody or antigen-binding fragment thereof comprises a CDR-L2 domain comprising or consisting of a sequence as substantially set out in SEQ ID No: 57, or a variant or fragment thereof. Preferably, the antibody or antigen-binding fragment thereof comprises a CDR-L3 domain comprising or consisting of a sequence as substantially set out in SEQ ID No: 58, or a variant or fragment thereof.

Preferably, the antibody or antigen-binding fragment thereof comprises a CDR-L1 domain comprising or consisting of SEQ ID No: 56, a CDR-L2 domain comprising or consisting of SEQ ID No: 57, and/or a CDR-L3 domain comprising or consisting of SEQ ID No: 58. However, preferably the antibody or antigen-binding fragment thereof comprises a CDR-L1 domain comprising or consisting of SEQ ID No: 56, a CDR-L2 domain comprising or consisting of SEQ ID No: 57, and a CDR-L3 domain comprising or consisting of SEQ ID No: 58.

Preferably, the antibody or antigen-binding fragment thereof comprises a light chain variable region comprising or consisting of a sequence as substantially set out in SEQ ID No: 59, or a variant or fragment thereof.

Preferably, the antibody or antigen-binding fragment thereof comprises a light chain variable (VL) region encoded by a nucleic acid sequence as substantially set out in SEQ ID No: 81, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 52, a CDR-H2 domain comprising or consisting of SEQ ID No: 53; a CDR-H3 domain comprising or consisting of SEQ ID No: 54, a CDR-L1 domain comprising or consisting of SEQ ID No: 56, a CDR-L2 domain comprising or consisting of SEQ ID No: 57, and a CDR-L3 domain comprising or consisting of SEQ ID No: 58.

Preferably, the antibody or antigen-binding fragment thereof comprises a heavy chain variable region comprising or consisting of SEQ ID No: 55, and a light chain variable region comprising or consisting of SEQ ID No: 59.

Preferably, the antibody or antigen-binding fragment thereof comprises a heavy chain variable region encoded by a nucleic acid sequence comprising or consisting of SEQ ID No: 80, and a light chain variable region encoded by a nucleic acid sequence comprising or consisting of SEQ ID No: 81.

Preferably, the antibody or antigen-binding fragment thereof comprises a heavy chain constant (HC) region comprising or consisting of a sequence as substantially set out in SEQ ID No: 60, or a variant or fragment thereof.

Preferably, the antibody or antigen-binding fragment thereof comprises a light chain constant (LC) region comprising or consisting of a sequence as substantially set out in SEQ ID No: 61, or a variant or fragment thereof.

Preferably, the antibody or antigen-binding fragment thereof comprises a heavy chain constant region comprising or consisting of SEQ ID No: 60 and a light chain constant region comprising or consisting of SEQ ID No: 61.

### 33D03 VH-1 VL-1

Alternatively, in another embodiment, the antibody or antigen-binding fragment thereof is a humanised antibody. In one embodiment, the humanised antibody or antigen-binding fragment thereof is referred to herein as 33D03_VH-1_VL-1.

Preferably, the antibody or antigen-binding fragment thereof comprises a CDR-H1 domain comprising or consisting of a sequence as substantially set out in SEQ ID No: 84, or a variant or fragment thereof. Preferably, the antibody or antigen-binding fragment thereof comprises a CDR-H2 domain comprising or consisting of a sequence as substantially set out in SEQ ID No: 85, or a variant or fragment thereof. Preferably, the antibody or antigen-binding fragment thereof comprises a CDR-H3 domain comprising or consisting of a sequence as substantially set out in SEQ ID No: 86, or a variant or fragment thereof.

Preferably, the antibody or antigen-binding fragment thereof comprises a CDR-H1 domain comprising or consisting of SEQ ID No: 84, a CDR-H2 domain comprising or consisting of SEQ ID No: 85 and/or a CDR-H3 domain comprising or consisting of SEQ ID No: 86. Preferably, however, the antibody or antigen-binding fragment thereof comprises a CDR-H1 domain comprising or consisting of SEQ ID No: 84, a CDR-H2 domain comprising or consisting of SEQ ID No: 85 and a CDR-H3 domain comprising or consisting of SEQ ID No: 86.

Preferably, the antibody or antigen-binding fragment thereof comprises a heavy chain variable (VH) region comprising or consisting of a sequence as substantially set out in SEQ ID No: 90, or a variant or fragment thereof.

Preferably, the antibody or antigen-binding fragment thereof comprises a CDR-L1 domain comprising or consisting of a sequence as substantially set out in SEQ ID No: 87, or a variant or fragment thereof. Preferably, the antibody or antigen-binding fragment thereof comprises a CDR-L2 domain comprising or consisting of a sequence as substantially set out in SEQ ID No: 88, or a variant or fragment thereof. Preferably, the antibody or antigen-binding fragment thereof comprises a CDR-L3 domain comprising or consisting of a sequence as substantially set out in SEQ ID No: 89, or a variant or fragment thereof.

Preferably, the antibody or antigen-binding fragment thereof comprises a CDR-L1 domain comprising or consisting of SEQ ID No: 87, a CDR-L2 domain comprising or consisting of SEQ ID No: 88, and/or a CDR-L3 domain comprising or consisting of SEQ ID No: 89. However, preferably the antibody or antigen-binding fragment thereof comprises a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a light chain variable region comprising or consisting of a sequence as substantially set out in SEQ ID No: 91, or a variant or fragment thereof.

Preferably, the antibody or antigen-binding fragment thereof comprises at least one, at antibody or antigen-binding fragment thereof comprises at least CDR-H3.

Preferably, the antibody or antigen-binding fragment thereof comprises a CDR-H1 domain comprising or consisting of SEQ ID No: 84, a CDR-H2 domain comprising or consisting of SEQ ID No: 85; a CDR-H3 domain comprising or consisting of SEQ ID No: 86, a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a heavy chain variable region comprising or consisting of SEQ ID No: 90, and a light chain variable region comprising or consisting of SEQ ID No: 91.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 33D03 VH-1 VL-2

Alternatively, in another embodiment, the antibody or antigen-binding fragment thereof is a humanised antibody. In one embodiment, the humanised antibody or antigen-binding fragment thereof is referred to herein as 33D03_VH-1_VL-2.

Preferably, the antibody or antigen-binding fragment thereof comprises a CDR-H1 domain comprising or consisting of a sequence as substantially set out in SEQ ID No: 84, or a variant or fragment thereof. Preferably, the antibody or antigen-binding fragment thereof comprises a CDR-H2 domain comprising or consisting of a sequence as substantially set out in SEQ ID No: 85, or a variant or fragment thereof. Preferably, the antibody or antigen-binding fragment thereof comprises a CDR-H3 domain comprising or consisting of a sequence as substantially set out in SEQ ID No: 86, or a variant or fragment thereof.

Preferably, the antibody or antigen-binding fragment thereof comprises a CDR-H1 domain comprising or consisting of SEQ ID No: 84, a CDR-H2 domain comprising or consisting of SEQ ID No: 85 and/or a CDR-H3 domain comprising or consisting of SEQ ID No: 86. Preferably, however, the antibody or antigen-binding fragment thereof comprises a CDR-H1 domain comprising or consisting of SEQ ID No: 84, a CDR-H2 domain comprising or consisting of SEQ ID No: 85 and a CDR-H3 domain comprising or consisting of SEQ ID No: 86.

Preferably, the antibody or antigen-binding fragment thereof comprises a heavy chain variable (VH) region comprising or consisting of a sequence as substantially set out in SEQ ID No: 90, or a variant or fragment thereof.

Preferably, the antibody or antigen-binding fragment thereof comprises a CDR-L1 domain comprising or consisting of a sequence as substantially set out in SEQ ID No: 87, or a variant or fragment thereof. Preferably, the antibody or antigen-binding fragment thereof comprises a CDR-L2 domain comprising or consisting of a sequence as substantially set out in SEQ ID No: 88, or a variant or fragment thereof. Preferably, the antibody or antigen-binding fragment thereof comprises a CDR-L3 domain comprising or consisting of a sequence as substantially set out in SEQ ID No: 89, or a variant or fragment thereof.

Preferably, the antibody or antigen-binding fragment thereof comprises a CDR-L1 domain comprising or consisting of SEQ ID No: 87, a CDR-L2 domain comprising or consisting of SEQ ID No: 88, and/or a CDR-L3 domain comprising or consisting of SEQ ID No: 89. However, preferably the antibody or antigen-binding fragment thereof comprises a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a light chain variable region comprising or consisting of a sequence as substantially set out in SEQ ID No: 94, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 84, a CDR-H2 domain comprising or consisting of SEQ ID No: 85; a CDR-H3 domain comprising or consisting of SEQ ID No: 86, a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a heavy chain variable region comprising or consisting of SEQ ID No: 90, and a light chain variable region comprising or consisting of SEQ ID No: 94.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 33D03 VH-1 VL-3

Alternatively, in another embodiment, the antibody or antigen-binding fragment thereof is a humanised antibody. In one embodiment, the humanised antibody or antigen-binding fragment thereof is referred to herein as 33D03_VH-1_VL-3.

Preferably, the antibody or antigen-binding fragment thereof comprises a CDR-H1 domain comprising or consisting of a sequence as substantially set out in SEQ ID No: 84, or a variant or fragment thereof. Preferably, the antibody or antigen-binding fragment thereof comprises a CDR-H2 domain comprising or consisting of a sequence as substantially set out in SEQ ID No: 85, or a variant or fragment thereof. Preferably, the antibody or antigen-binding fragment thereof comprises a CDR-H3 domain comprising or consisting of a sequence as substantially set out in SEQ ID No: 86, or a variant or fragment thereof.

Preferably, the antibody or antigen-binding fragment thereof comprises a CDR-H1 domain comprising or consisting of SEQ ID No: 84, a CDR-H2 domain comprising or consisting of SEQ ID No: 85 and/or a CDR-H3 domain comprising or consisting of SEQ ID No: 86. Preferably, however, the antibody or antigen-binding fragment thereof comprises a CDR-H1 domain comprising or consisting of SEQ ID No: 84, a CDR-H2 domain comprising or consisting of SEQ ID No: 85 and a CDR-H3 domain comprising or consisting of SEQ ID No: 86.

Preferably, the antibody or antigen-binding fragment thereof comprises a heavy chain variable (VH) region comprising or consisting of a sequence as substantially set out in SEQ ID No: 90, or a variant or fragment thereof.

Preferably, the antibody or antigen-binding fragment thereof comprises a CDR-L1 domain comprising or consisting of a sequence as substantially set out in SEQ ID No: 87, or a variant or fragment thereof. Preferably, the antibody or antigen-binding fragment thereof comprises a CDR-L2 domain comprising or consisting of a sequence as substantially set out in SEQ ID No: 88, or a variant or fragment thereof. Preferably, the antibody or antigen-binding fragment thereof comprises a CDR-L3 domain comprising or consisting of a sequence as substantially set out in SEQ ID No: 89, or a variant or fragment thereof.

Preferably, the antibody or antigen-binding fragment thereof comprises a CDR-L1 domain comprising or consisting of SEQ ID No: 87, a CDR-L2 domain comprising or consisting of SEQ ID No: 88, and/or a CDR-L3 domain comprising or consisting of SEQ ID No: 89. However, preferably the antibody or antigen-binding fragment thereof comprises a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a light chain variable region comprising or consisting of a sequence as substantially set out in SEQ ID No: 95, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 84, a CDR-H2 domain comprising or consisting of SEQ ID No: 85; a CDR-H3 domain comprising or consisting of SEQ ID No: 86, a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a heavy chain variable region comprising or consisting of SEQ ID No: 90, and a light chain variable region comprising or consisting of SEQ ID No: 95.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 33D03 VH-1 VL-4

Alternatively, in another embodiment, the antibody or antigen-binding fragment antigen-binding fragment thereof is referred to herein as 33D03_VH-1_VL-4.

Preferably, the antibody or antigen-binding fragment thereof comprises a CDR-H1 domain comprising or consisting of a sequence as substantially set out in SEQ ID No: 84, or a variant or fragment thereof. Preferably, the antibody or antigen-binding fragment thereof comprises a CDR-H2 domain comprising or consisting of a sequence as substantially set out in SEQ ID No: 85, or a variant or fragment thereof. Preferably, the antibody or antigen-binding fragment thereof comprises a CDR-H3 domain comprising or consisting of a sequence as substantially set out in SEQ ID No: 86, or a variant or fragment thereof.

Preferably, the antibody or antigen-binding fragment thereof comprises a CDR-H1 domain comprising or consisting of SEQ ID No: 84, a CDR-H2 domain comprising or consisting of SEQ ID No: 85 and/or a CDR-H3 domain comprising or consisting of SEQ ID No: 86. Preferably, however, the antibody or antigen-binding fragment thereof comprises a CDR-H1 domain comprising or consisting of SEQ ID No: 84, a CDR-H2 domain comprising or consisting of SEQ ID No: 85 and a CDR-H3 domain comprising or consisting of SEQ ID No: 86.

Preferably, the antibody or antigen-binding fragment thereof comprises a heavy chain variable (VH) region comprising or consisting of a sequence as substantially set out in SEQ ID No: 90, or a variant or fragment thereof.

Preferably, the antibody or antigen-binding fragment thereof comprises a CDR-L1 domain comprising or consisting of a sequence as substantially set out in SEQ ID No: 87, or a variant or fragment thereof. Preferably, the antibody or antigen-binding fragment thereof comprises a CDR-L2 domain comprising or consisting of a sequence as substantially set out in SEQ ID No: 88, or a variant or fragment thereof. Preferably, the antibody or antigen-binding fragment thereof comprises a CDR-L3 domain comprising or consisting of a sequence as substantially set out in SEQ ID No: 89, or a variant or fragment thereof.

Preferably, the antibody or antigen-binding fragment thereof comprises a CDR-L1 domain comprising or consisting of SEQ ID No: 87, a CDR-L2 domain comprising or consisting of SEQ ID No: 88, and/or a CDR-L3 domain comprising or consisting of SEQ ID No: 89. However, preferably the antibody or antigen-binding fragment thereof comprises a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a light chain variable region comprising or consisting of a sequence as substantially set out in SEQ ID No: 96, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 84, a CDR-H2 domain comprising or consisting of SEQ ID No: 85; a CDR-H3 domain comprising or consisting of SEQ ID No: 86, a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a heavy chain variable region comprising or consisting of SEQ ID No: 90, and a light chain variable region comprising or consisting of SEQ ID No: 96.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 33D03 VH-2 VL-1

Alternatively, in another embodiment, the antibody or antigen-binding fragment thereof is a humanised antibody. In one embodiment, the humanised antibody or antigen-binding fragment thereof is referred to herein as 33D03_VH-2_VL-1.

Preferably, the antibody or antigen-binding fragment thereof comprises a CDR-H1 domain comprising or consisting of a sequence as substantially set out in SEQ ID No: 84, or a variant or fragment thereof. Preferably, the antibody or antigen-binding fragment thereof comprises a CDR-H2 domain comprising or consisting of a sequence as substantially set out in SEQ ID No: 85, or a variant or fragment thereof. Preferably, the antibody or antigen-binding fragment thereof comprises a CDR-H3 domain comprising or consisting of a sequence as substantially set out in SEQ ID No: 86, or a variant or fragment thereof.

Preferably, the antibody or antigen-binding fragment thereof comprises a CDR-H1 domain comprising or consisting of SEQ ID No: 84, a CDR-H2 domain comprising or consisting of SEQ ID No: 85 and/or a CDR-H3 domain comprising or consisting of SEQ ID No: 86. Preferably, however, the antibody or antigen-binding fragment thereof comprises a CDR-H1 domain comprising or consisting of SEQ ID No: 84, a CDR-H2 domain comprising or consisting of SEQ ID No: 85 and a CDR-H3 domain comprising or consisting of SEQ ID No: 86.

Preferably, the antibody or antigen-binding fragment thereof comprises a heavy chain variable (VH) region comprising or consisting of a sequence as substantially set out in SEQ ID No: 97, or a variant or fragment thereof.

Preferably, the antibody or antigen-binding fragment thereof comprises a CDR-L1 domain comprising or consisting of a sequence as substantially set out in SEQ ID No: 87, or a variant or fragment thereof. Preferably, the antibody or antigen-binding fragment thereof comprises a CDR-L2 domain comprising or consisting of a sequence as substantially set out in SEQ ID No: 88, or a variant or fragment thereof. Preferably, the antibody or antigen-binding fragment thereof comprises a CDR-L3 domain comprising or consisting of a sequence as substantially set out in SEQ ID No: 89, or a

Preferably, the antibody or antigen-binding fragment thereof comprises a CDR-L1 domain comprising or consisting of SEQ ID No: 87, a CDR-L2 domain comprising or consisting of SEQ ID No: 88, and/or a CDR-L3 domain comprising or consisting of SEQ ID No: 89. However, preferably the antibody or antigen-binding fragment thereof comprises a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a light chain variable region comprising or consisting of a sequence as substantially set out in SEQ ID No: 91, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 84, a CDR-H2 domain comprising or consisting of SEQ ID No: 85; a CDR-H3 domain comprising or consisting of SEQ ID No: 86, a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a heavy chain variable region comprising or consisting of SEQ ID No: 97, and a light chain variable region comprising or consisting of SEQ ID No: 91.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 33D03 VH-2 VL-2

Alternatively, in another embodiment, the antibody or antigen-binding fragment thereof is a humanised antibody. In one embodiment, the humanised antibody or antigen-binding fragment thereof is referred to herein as 33D03_VH-2_VL-2.

Preferably, the antibody or antigen-binding fragment thereof comprises a CDR-H1 domain comprising or consisting of a sequence as substantially set out in SEQ ID No: 84, or a variant or fragment thereof. Preferably, the antibody or antigen-binding fragment thereof comprises a CDR-H2 domain comprising or consisting of a sequence as substantially set out in SEQ ID No: 85, or a variant or fragment thereof. Preferably, the antibody or antigen-binding fragment thereof comprises a CDR-H3 domain comprising or consisting of a sequence as substantially set out in SEQ ID No: 86, or a variant or fragment thereof.

Preferably, the antibody or antigen-binding fragment thereof comprises a CDR-H1 domain comprising or consisting of SEQ ID No: 84, a CDR-H2 domain comprising or consisting of SEQ ID No: 85 and/or a CDR-H3 domain comprising or consisting of SEQ ID No: 86. Preferably, however, the antibody or antigen-binding fragment thereof comprises a CDR-H1 domain comprising or consisting of SEQ ID No: 84, a CDR-H2 domain comprising or consisting of SEQ ID No: 85 and a CDR-H3 domain comprising or consisting of SEQ ID No: 86.

Preferably, the antibody or antigen-binding fragment thereof comprises a heavy chain variable (VH) region comprising or consisting of a sequence as substantially set out in SEQ ID No: 97, or a variant or fragment thereof.

Preferably, the antibody or antigen-binding fragment thereof comprises a CDR-L1 domain comprising or consisting of a sequence as substantially set out in SEQ ID No: 87, or a variant or fragment thereof. Preferably, the antibody or antigen-binding fragment thereof comprises a CDR-L2 domain comprising or consisting of a sequence as substantially set out in SEQ ID No: 88, or a variant or fragment thereof. Preferably, the antibody or antigen-binding fragment thereof comprises a CDR-L3 domain comprising or consisting of a sequence as substantially set out in SEQ ID No: 89, or a variant or fragment thereof.

Preferably, the antibody or antigen-binding fragment thereof comprises a CDR-L1 domain comprising or consisting of SEQ ID No: 87, a CDR-L2 domain comprising or consisting of SEQ ID No: 88, and/or a CDR-L3 domain comprising or consisting of SEQ ID No: 89. However, preferably the antibody or antigen-binding fragment thereof comprises a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a light chain variable region comprising or consisting of a sequence as substantially set out in SEQ ID No: 94, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 84, a CDR-H2 domain comprising or consisting of SEQ ID No: 85; a CDR-H3 domain comprising or consisting of SEQ ID No: 86, a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a heavy chain variable region comprising or consisting of SEQ ID No: 97, and a light chain variable region comprising or consisting of SEQ ID No: 94.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 33D03 VH-2 VL-3

Alternatively, in another embodiment, the antibody or antigen-binding fragment thereof is a humanised antibody. In one embodiment, the humanised antibody or antigen-binding fragment thereof is referred to herein as 33D03_VH-2_VL-3.

Preferably, the antibody or antigen-binding fragment thereof comprises a CDR-H1 domain comprising or consisting of a sequence as substantially set out in SEQ ID No: 84, or a variant or fragment thereof. Preferably, the antibody or antigen-binding fragment thereof comprises a CDR-H2 domain comprising or consisting of a sequence as substantially set out in SEQ ID No: 85, or a variant or fragment thereof. Preferably, the antibody or antigen-binding fragment thereof comprises a CDR-H3 domain comprising or consisting of a sequence as substantially set out in SEQ ID No: 86, or a variant or fragment thereof.

Preferably, the antibody or antigen-binding fragment thereof comprises a CDR-H1 domain comprising or consisting of SEQ ID No: 84, a CDR-H2 domain comprising or consisting of SEQ ID No: 85 and/or a CDR-H3 domain comprising or consisting of SEQ ID No: 86. Preferably, however, the antibody or antigen-binding fragment thereof comprises a CDR-H1 domain comprising or consisting of SEQ ID No: 84, a CDR-H2 domain comprising or consisting of SEQ ID No: 85 and a CDR-H3 domain comprising or consisting of SEQ ID No: 86.

Preferably, the antibody or antigen-binding fragment thereof comprises a heavy chain variable (VH) region comprising or consisting of a sequence as substantially set out in SEQ ID No: 97, or a variant or fragment thereof.

Preferably, the antibody or antigen-binding fragment thereof comprises a CDR-L1 domain comprising or consisting of a sequence as substantially set out in SEQ ID No: 87, or a variant or fragment thereof. Preferably, the antibody or antigen-binding fragment thereof comprises a CDR-L2 domain comprising or consisting of a sequence as substantially set out in SEQ ID No: 88, or a variant or fragment thereof. Preferably, the antibody or antigen-binding fragment thereof comprises a CDR-L3 domain comprising or consisting of a sequence as substantially set out in SEQ ID No: 89, or a variant or fragment thereof.

Preferably, the antibody or antigen-binding fragment thereof comprises a CDR-L1 domain comprising or consisting of SEQ ID No: 87, a CDR-L2 domain comprising or consisting of SEQ ID No: 88, and/or a CDR-L3 domain comprising or consisting of SEQ ID No: 89. However, preferably the antibody or antigen-binding fragment thereof comprises a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a light chain variable region comprising or consisting of a sequence as substantially set out in SEQ ID No: 95, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 84, a CDR-H2 domain comprising or consisting of SEQ ID No: 85; a CDR-H3 domain comprising or consisting of SEQ ID No: 86, a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a heavy chain variable region comprising or consisting of SEQ ID No: 97, and a light chain variable region comprising or consisting of SEQ ID No: 95.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 33D03 VH-2 VL-4

Alternatively, in another embodiment, the antibody or antigen-binding fragment thereof is a humanised antibody. In one embodiment, the humanised antibody or antigen-binding fragment thereof is referred to herein as 33D03_VH-2_VL-4.

Preferably, the antibody or antigen-binding fragment thereof comprises a CDR-H1 domain comprising or consisting of a sequence as substantially set out in SEQ ID No: 84, or a variant or fragment thereof. Preferably, the antibody or antigen-binding fragment thereof comprises a CDR-H2 domain comprising or consisting of a sequence as substantially set out in SEQ ID No: 85, or a variant or fragment thereof. Preferably, the antibody or antigen-binding fragment thereof comprises a CDR-H3 domain comprising or consisting of a sequence as substantially set out in SEQ ID No: 86, or a variant or fragment thereof.

Preferably, the antibody or antigen-binding fragment thereof comprises a CDR-H1 domain comprising or consisting of SEQ ID No: 84, a CDR-H2 domain comprising or consisting of SEQ ID No: 85 and/or a CDR-H3 domain comprising or consisting of SEQ ID No: 86. Preferably, however, the antibody or antigen-binding fragment thereof comprises a CDR-H1 domain comprising or consisting of SEQ ID No: 84, a CDR-H2 domain comprising or consisting of SEQ ID No: 85 and a CDR-H3 domain comprising or consisting of SEQ ID No: 86.

Preferably, the antibody or antigen-binding fragment thereof comprises a heavy chain variable (VH) region comprising or consisting of a sequence as substantially set out in SEQ ID No: 97, or a variant or fragment thereof.

Preferably, the antibody or antigen-binding fragment thereof comprises a CDR-L1 domain comprising or consisting of a sequence as substantially set out in SEQ ID No: 87, or a variant or fragment thereof. Preferably, the antibody or antigen-binding fragment thereof comprises a CDR-L2 domain comprising or consisting of a sequence as substantially set out in SEQ ID No: 88, or a variant or fragment thereof. Preferably, the antibody or antigen-binding fragment thereof comprises a CDR-L3 domain comprising or consisting of a sequence as substantially set out in SEQ ID No: 89, or a variant or fragment thereof.

Preferably, the antibody or antigen-binding fragment thereof comprises a CDR-L1 domain comprising or consisting of SEQ ID No: 87, a CDR-L2 domain comprising or consisting of SEQ ID No: 88, and/or a CDR-L3 domain comprising or consisting of SEQ ID No: 89. However, preferably the antibody or antigen-binding fragment thereof comprises a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a light chain variable region comprising or consisting of a sequence as substantially set out in SEQ ID No: 96, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 84, a CDR-H2 domain comprising or consisting of SEQ ID No: 85; a CDR-H3 domain comprising or consisting of SEQ ID No: 86, a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a heavy chain variable region comprising or consisting of SEQ ID No: 97, and a light chain variable region comprising or consisting of SEQ ID No: 96.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 33D03 VH-3 VL-1

Alternatively, in another embodiment, the antibody or antigen-binding fragment thereof is a humanised antibody. In one embodiment, the humanised antibody or antigen-binding fragment thereof is referred to herein as 33D03_VH-3_VL-1.

Preferably, the antibody or antigen-binding fragment thereof comprises a CDR-H1 domain comprising or consisting of a sequence as substantially set out in SEQ ID No: 84, or a variant or fragment thereof. Preferably, the antibody or antigen-binding fragment thereof comprises a CDR-H2 domain comprising or consisting of a sequence as substantially set out in SEQ ID No: 85, or a variant or fragment thereof. Preferably, the antibody or antigen-binding fragment thereof comprises a CDR-H3 domain comprising or consisting of a sequence as substantially set out in SEQ ID No: 86, or a variant or fragment thereof.

Preferably, the antibody or antigen-binding fragment thereof comprises a CDR-H1 domain comprising or consisting of SEQ ID No: 84, a CDR-H2 domain comprising or consisting of SEQ ID No: 85 and/or a CDR-H3 domain comprising or consisting of SEQ ID No: 86. Preferably, however, the antibody or antigen-binding fragment thereof comprises a CDR-H1 domain comprising or consisting of SEQ ID No: 84, a CDR-H2 domain comprising or consisting of SEQ ID No: 85 and a CDR-H3 domain comprising or consisting of SEQ ID No: 86.

Preferably, the antibody or antigen-binding fragment thereof comprises a heavy chain variable (VH) region comprising or consisting of a sequence as substantially set out in SEQ ID No: 98, or a variant or fragment thereof.

Preferably, the antibody or antigen-binding fragment thereof comprises a CDR-L1 domain comprising or consisting of a sequence as substantially set out in SEQ ID No: 87, or a variant or fragment thereof. Preferably, the antibody or antigen-binding fragment thereof comprises a CDR-L2 domain comprising or consisting of a sequence as substantially set out in SEQ ID No: 88, or a variant or fragment thereof. Preferably, the antibody or antigen-binding fragment thereof comprises a CDR-L3 domain comprising or consisting of a sequence as substantially set out in SEQ ID No: 89, or a variant or fragment thereof.

Preferably, the antibody or antigen-binding fragment thereof comprises a CDR-L1 domain comprising or consisting of SEQ ID No: 87, a CDR-L2 domain comprising or consisting of SEQ ID No: 88, and/or a CDR-L3 domain comprising or consisting of SEQ ID No: 89. However, preferably the antibody or antigen-binding fragment thereof comprises a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a light chain variable region comprising or consisting of a sequence as substantially set out in SEQ ID No: 91, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 84, a CDR-H2 domain comprising or consisting of SEQ ID No: 85; a CDR-H3 domain comprising or consisting of SEQ ID No: 86, a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a heavy chain variable region comprising or consisting of SEQ ID No: 98, and a light chain variable region comprising or consisting of SEQ ID No: 91.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 33D03 VH-3 VL-2

Alternatively, in another embodiment, the antibody or antigen-binding fragment thereof is a humanised antibody. In one embodiment, the humanised antibody or antigen-binding fragment thereof is referred to herein as 33D03_VH-3_VL-2.

Preferably, the antibody or antigen-binding fragment thereof comprises a CDR-H1 domain comprising or consisting of a sequence as substantially set out in SEQ ID No: 84, or a variant or fragment thereof. Preferably, the antibody or antigen-binding fragment thereof comprises a CDR-H2 domain comprising or consisting of a sequence as substantially set out in SEQ ID No: 85, or a variant or fragment thereof. Preferably, the antibody or antigen-binding fragment thereof comprises a CDR-H3 domain comprising or consisting of a sequence as substantially set out in SEQ ID No: 86, or a variant or fragment thereof.

Preferably, the antibody or antigen-binding fragment thereof comprises a CDR-H1 domain comprising or consisting of SEQ ID No: 84, a CDR-H2 domain comprising or consisting of SEQ ID No: 85 and/or a CDR-H3 domain comprising or consisting of SEQ ID No: 86. Preferably, however, the antibody or antigen-binding fragment thereof comprises a CDR-H1 domain comprising or consisting of SEQ ID No: 84, a CDR-H2 domain comprising or consisting of SEQ ID No: 85 and a CDR-H3 domain comprising or consisting of SEQ ID No: 86.

Preferably, the antibody or antigen-binding fragment thereof comprises a heavy chain variable (VH) region comprising or consisting of a sequence as substantially set out in SEQ ID No: 98, or a variant or fragment thereof.

Preferably, the antibody or antigen-binding fragment thereof comprises a CDR-L1 domain comprising or consisting of a sequence as substantially set out in SEQ ID No: 87, or a variant or fragment thereof. Preferably, the antibody or antigen-binding fragment thereof comprises a CDR-L2 domain comprising or consisting of a sequence as substantially set out in SEQ ID No: 88, or a variant or fragment thereof. Preferably, the antibody or antigen-binding fragment thereof comprises a CDR-L3 domain comprising or consisting of a sequence as substantially set out in SEQ ID No: 89, or a variant or fragment thereof.

Preferably, the antibody or antigen-binding fragment thereof comprises a CDR-L1 domain comprising or consisting of SEQ ID No: 87, a CDR-L2 domain comprising or consisting of SEQ ID No: 88, and/or a CDR-L3 domain comprising or consisting of SEQ ID No: 89. However, preferably the antibody or antigen-binding fragment thereof comprises a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a light chain variable region comprising or consisting of a sequence as substantially set out in SEQ ID No: 94, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 84, a CDR-H2 domain comprising or consisting of SEQ ID No: 85; a CDR-H3 domain comprising or consisting of SEQ ID No: 86, a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a heavy chain variable region comprising or consisting of SEQ ID No: 98, and a light chain variable region comprising or consisting of SEQ ID No: 94.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 33D03 VH-3 VL-3

Alternatively, in another embodiment, the antibody or antigen-binding fragment thereof is a humanised antibody. In one embodiment, the humanised antibody or antigen-binding fragment thereof is referred to herein as 33D03_VH-3_VL-3.

Preferably, the antibody or antigen-binding fragment thereof comprises a CDR-H1 domain comprising or consisting of a sequence as substantially set out in SEQ ID No: 84, or a variant or fragment thereof. Preferably, the antibody or antigen-binding fragment thereof comprises a CDR-H2 domain comprising or consisting of a sequence as substantially set out in SEQ ID No: 85, or a variant or fragment thereof. Preferably, the antibody or antigen-binding fragment thereof comprises a CDR-H3 domain comprising or consisting of a sequence as substantially set out in SEQ ID No: 86, or a variant or fragment thereof.

Preferably, the antibody or antigen-binding fragment thereof comprises a CDR-H1 domain comprising or consisting of SEQ ID No: 84, a CDR-H2 domain comprising or consisting of SEQ ID No: 85 and/or a CDR-H3 domain comprising or consisting of SEQ ID No: 86. Preferably, however, the antibody or antigen-binding fragment thereof comprises a CDR-H1 domain comprising or consisting of SEQ ID No: 84, a CDR-H2 domain comprising or consisting of SEQ ID No: 85 and a CDR-H3 domain comprising or consisting of SEQ ID No: 86.

Preferably, the antibody or antigen-binding fragment thereof comprises a heavy chain variable (VH) region comprising or consisting of a sequence as substantially set out in SEQ ID No: 98, or a variant or fragment thereof.

Preferably, the antibody or antigen-binding fragment thereof comprises a CDR-L1 domain comprising or consisting of a sequence as substantially set out in SEQ ID No: 87, or a variant or fragment thereof. Preferably, the antibody or antigen-binding fragment thereof comprises a CDR-L2 domain comprising or consisting of a sequence as substantially set out in SEQ ID No: 88, or a variant or fragment thereof. Preferably, the antibody or antigen-binding fragment thereof comprises a CDR-L3 domain comprising or consisting of a sequence as substantially set out in SEQ ID No: 89, or a variant or fragment thereof.

Preferably, the antibody or antigen-binding fragment thereof comprises a CDR-L1 domain comprising or consisting of SEQ ID No: 87, a CDR-L2 domain comprising or consisting of SEQ ID No: 88, and/or a CDR-L3 domain comprising or consisting of SEQ ID No: 89. However, preferably the antibody or antigen-binding fragment thereof comprises a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a light chain variable region comprising or consisting of a sequence as substantially set out in SEQ ID No: 95, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 84, a CDR-H2 domain comprising or consisting of SEQ ID No: 85; a CDR-H3 domain comprising or consisting of SEQ ID No: 86, a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a heavy chain variable region comprising or consisting of SEQ ID No: 98, and a light chain variable region comprising or consisting of SEQ ID No: 95.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 33D03 VH-3 VL-4

Alternatively, in another embodiment, the antibody or antigen-binding fragment thereof is a humanised antibody. In one embodiment, the humanised antibody or antigen-binding fragment thereof is referred to herein as 33D03_VH-3_VL-4.

Preferably, the antibody or antigen-binding fragment thereof comprises a CDR-H1 domain comprising or consisting of a sequence as substantially set out in SEQ ID No: 84, or a variant or fragment thereof. Preferably, the antibody or antigen-binding fragment thereof comprises a CDR-H2 domain comprising or consisting of a sequence as substantially set out in SEQ ID No: 85, or a variant or fragment thereof. Preferably, the antibody or antigen-binding fragment thereof comprises a CDR-H3 domain comprising or consisting of a sequence as substantially set out in SEQ ID No: 86, or a variant or fragment thereof.

Preferably, the antibody or antigen-binding fragment thereof comprises a CDR-H1 domain comprising or consisting of SEQ ID No: 84, a CDR-H2 domain comprising or consisting of SEQ ID No: 85 and/or a CDR-H3 domain comprising or consisting of SEQ ID No: 86. Preferably, however, the antibody or antigen-binding fragment thereof comprises a CDR-H1 domain comprising or consisting of SEQ ID No: 84, a CDR-H2 domain comprising or consisting of SEQ ID No: 85 and a CDR-H3 domain comprising or consisting of SEQ ID No: 86.

Preferably, the antibody or antigen-binding fragment thereof comprises a heavy chain variable (VH) region comprising or consisting of a sequence as substantially set out in SEQ ID No: 98, or a variant or fragment thereof.

Preferably, the antibody or antigen-binding fragment thereof comprises a CDR-L1 domain comprising or consisting of a sequence as substantially set out in SEQ ID No: 87, or a variant or fragment thereof. Preferably, the antibody or antigen-binding fragment thereof comprises a CDR-L2 domain comprising or consisting of a sequence as substantially set out in SEQ ID No: 88, or a variant or fragment thereof. Preferably, the antibody or antigen-binding fragment thereof comprises a CDR-L3 domain comprising or consisting of a sequence as substantially set out in SEQ ID No: 89, or a variant or fragment thereof.

Preferably, the antibody or antigen-binding fragment thereof comprises a CDR-L1 domain comprising or consisting of SEQ ID No: 87, a CDR-L2 domain comprising or consisting of SEQ ID No: 88, and/or a CDR-L3 domain comprising or consisting of SEQ ID No: 89. However, preferably the antibody or antigen-binding fragment thereof comprises a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a light chain variable region comprising or consisting of a sequence as substantially set out in SEQ ID No: 96, or a variant or fragment thereof.

Preferably, the antibody or antigen-binding fragment thereof comprises at least one, at antibody or antigen-binding fragment thereof comprises at least CDR-H3.

Preferably, the antibody or antigen-binding fragment thereof comprises a CDR-H1 domain comprising or consisting of SEQ ID No: 84, a CDR-H2 domain comprising or consisting of SEQ ID No: 85; a CDR-H3 domain comprising or consisting of SEQ ID No: 86, a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a heavy chain variable region comprising or consisting of SEQ ID No: 98, and a light chain variable region comprising or consisting of SEQ ID No: 96.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 33D03 VH-4 VL-1

Alternatively, in another embodiment, the antibody or antigen-binding fragment thereof is a humanised antibody. In one embodiment, the humanised antibody or antigen-binding fragment thereof is referred to herein as 33D03_VH-4_VL-1.

Preferably, the antibody or antigen-binding fragment thereof comprises a CDR-H1 domain comprising or consisting of a sequence as substantially set out in SEQ ID No: 84, or a variant or fragment thereof. Preferably, the antibody or antigen-binding fragment thereof comprises a CDR-H2 domain comprising or consisting of a sequence as substantially set out in SEQ ID No: 85, or a variant or fragment thereof. Preferably, the antibody or antigen-binding fragment thereof comprises a CDR-H3 domain comprising or consisting of a sequence as substantially set out in SEQ ID No: 86, or a variant or fragment thereof.

Preferably, the antibody or antigen-binding fragment thereof comprises a CDR-H1 domain comprising or consisting of SEQ ID No: 84, a CDR-H2 domain comprising or consisting of SEQ ID No: 85 and/or a CDR-H3 domain comprising or consisting of SEQ ID No: 86. Preferably, however, the antibody or antigen-binding fragment thereof comprises a CDR-H1 domain comprising or consisting of SEQ ID No: 84, a CDR-H2 domain comprising or consisting of SEQ ID No: 85 and a CDR-H3 domain comprising or consisting of SEQ ID No: 86.

Preferably, the antibody or antigen-binding fragment thereof comprises a heavy chain variable (VH) region comprising or consisting of a sequence as substantially set out in SEQ ID No: 99, or a variant or fragment thereof.

Preferably, the antibody or antigen-binding fragment thereof comprises a CDR-L1 domain comprising or consisting of a sequence as substantially set out in SEQ ID No: 87, or a variant or fragment thereof. Preferably, the antibody or antigen-binding fragment thereof comprises a CDR-L2 domain comprising or consisting of a sequence as substantially set out in SEQ ID No: 88, or a variant or fragment thereof. Preferably, the antibody or antigen-binding fragment thereof comprises a CDR-L3 domain comprising or consisting of a sequence as substantially set out in SEQ ID No: 89, or a variant or fragment thereof.

Preferably, the antibody or antigen-binding fragment thereof comprises a CDR-L1 domain comprising or consisting of SEQ ID No: 87, a CDR-L2 domain comprising or consisting of SEQ ID No: 88, and/or a CDR-L3 domain comprising or consisting of SEQ ID No: 89. However, preferably the antibody or antigen-binding fragment thereof comprises a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a light chain variable region comprising or consisting of a sequence as substantially set out in SEQ ID No: 91, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 84, a CDR-H2 domain comprising or consisting of SEQ ID No: 85; a CDR-H3 domain comprising or consisting of SEQ ID No: 86, a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a heavy chain variable region comprising or consisting of SEQ ID No: 99, and a light chain variable region comprising or consisting of SEQ ID No: 91.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 33D03 VH-4 VL-2

Alternatively, in another embodiment, the antibody or antigen-binding fragment thereof is a humanised antibody. In one embodiment, the humanised antibody or antigen-binding fragment thereof is referred to herein as 33D03_VH-4_VL-2.

Preferably, the antibody or antigen-binding fragment thereof comprises a CDR-H1 domain comprising or consisting of a sequence as substantially set out in SEQ ID No: 84, or a variant or fragment thereof. Preferably, the antibody or antigen-binding fragment thereof comprises a CDR-H2 domain comprising or consisting of a sequence as substantially set out in SEQ ID No: 85, or a variant or fragment thereof. Preferably, the antibody or antigen-binding fragment thereof comprises a CDR-H3 domain comprising or consisting of a sequence as substantially set out in SEQ ID No: 86, or a variant or fragment thereof.

Preferably, the antibody or antigen-binding fragment thereof comprises a CDR-H1 domain comprising or consisting of SEQ ID No: 84, a CDR-H2 domain comprising or consisting of SEQ ID No: 85 and/or a CDR-H3 domain comprising or consisting of SEQ ID No: 86. Preferably, however, the antibody or antigen-binding fragment thereof comprises a CDR-H1 domain comprising or consisting of SEQ ID No: 84, a CDR-H2 domain comprising or consisting of SEQ ID No: 85 and a CDR-H3 domain comprising or consisting of SEQ ID No: 86.

Preferably, the antibody or antigen-binding fragment thereof comprises a heavy chain variable (VH) region comprising or consisting of a sequence as substantially set out in SEQ ID No: 99, or a variant or fragment thereof.

Preferably, the antibody or antigen-binding fragment thereof comprises a CDR-L1 domain comprising or consisting of a sequence as substantially set out in SEQ ID No: 87, or a variant or fragment thereof. Preferably, the antibody or antigen-binding fragment thereof comprises a CDR-L2 domain comprising or consisting of a sequence as substantially set out in SEQ ID No: 88, or a variant or fragment thereof. Preferably, the antibody or antigen-binding fragment thereof comprises a CDR-L3 domain comprising or consisting of a sequence as substantially set out in SEQ ID No: 89, or a variant or fragment thereof.

Preferably, the antibody or antigen-binding fragment thereof comprises a CDR-L1 domain comprising or consisting of SEQ ID No: 87, a CDR-L2 domain comprising or consisting of SEQ ID No: 88, and/or a CDR-L3 domain comprising or consisting of SEQ ID No: 89. However, preferably the antibody or antigen-binding fragment thereof comprises a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a light chain variable region comprising or consisting of a sequence as substantially set out in SEQ ID No: 94, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 84, a CDR-H2 domain comprising or consisting of SEQ ID No: 85; a CDR-H3 domain comprising or consisting of SEQ ID No: 86, a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a heavy chain variable region comprising or consisting of SEQ ID No: 99, and a light chain variable region comprising or consisting of SEQ ID No: 94.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 33D03 VH-4 VL-3

Alternatively, in another embodiment, the antibody or antigen-binding fragment thereof is a humanised antibody. In one embodiment, the humanised antibody or antigen-binding fragment thereof is referred to herein as 33D03_VH-4_VL-3.

Preferably, the antibody or antigen-binding fragment thereof comprises a CDR-H1 domain comprising or consisting of a sequence as substantially set out in SEQ ID No: 84, or a variant or fragment thereof. Preferably, the antibody or antigen-binding fragment thereof comprises a CDR-H2 domain comprising or consisting of a sequence as substantially set out in SEQ ID No: 85, or a variant or fragment thereof. Preferably, the antibody or antigen-binding fragment thereof comprises a CDR-H3 domain comprising or consisting of a sequence as substantially set out in SEQ ID No: 86, or a variant or fragment thereof.

Preferably, the antibody or antigen-binding fragment thereof comprises a CDR-H1 domain comprising or consisting of SEQ ID No: 84, a CDR-H2 domain comprising or consisting of SEQ ID No: 85 and/or a CDR-H3 domain comprising or consisting of SEQ ID No: 86. Preferably, however, the antibody or antigen-binding fragment thereof comprises a CDR-H1 domain comprising or consisting of SEQ ID No: 84, a CDR-H2 domain comprising or consisting of SEQ ID No: 85 and a CDR-H3 domain comprising or consisting of SEQ ID No: 86.

Preferably, the antibody or antigen-binding fragment thereof comprises a heavy chain variable (VH) region comprising or consisting of a sequence as substantially set out in SEQ ID No: 99, or a variant or fragment thereof.

Preferably, the antibody or antigen-binding fragment thereof comprises a CDR-L1 domain comprising or consisting of a sequence as substantially set out in SEQ ID No: 87, or a variant or fragment thereof. Preferably, the antibody or antigen-binding fragment thereof comprises a CDR-L2 domain comprising or consisting of a sequence as substantially set out in SEQ ID No: 88, or a variant or fragment thereof. Preferably, the antibody or antigen-binding fragment thereof comprises a CDR-L3 domain comprising or consisting of a sequence as substantially set out in SEQ ID No: 89, or a variant or fragment thereof.

Preferably, the antibody or antigen-binding fragment thereof comprises a CDR-L1 domain comprising or consisting of SEQ ID No: 87, a CDR-L2 domain comprising or consisting of SEQ ID No: 88, and/or a CDR-L3 domain comprising or consisting of SEQ ID No: 89. However, preferably the antibody or antigen-binding fragment thereof comprises a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a light chain variable region comprising or consisting of a sequence as substantially set out in SEQ ID No: 95, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 84, a CDR-H2 domain comprising or consisting of SEQ ID No: 85; a CDR-H3 domain comprising or consisting of SEQ ID No: 86, a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a heavy chain variable region comprising or consisting of SEQ ID No: 99, and a light chain variable region comprising or consisting of SEQ ID No: 95.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 33D03 VH-4 VL-4

Alternatively, in another embodiment, the antibody or antigen-binding fragment thereof is a humanised antibody. In one embodiment, the humanised antibody or antigen-binding fragment thereof is referred to herein as 33D03_VH-4_VL-4.

Preferably, the antibody or antigen-binding fragment thereof comprises a CDR-H1 domain comprising or consisting of a sequence as substantially set out in SEQ ID No: 84, or a variant or fragment thereof. Preferably, the antibody or antigen-binding fragment thereof comprises a CDR-H2 domain comprising or consisting of a sequence as substantially set out in SEQ ID No: 85, or a variant or fragment thereof. Preferably, the antibody or antigen-binding fragment thereof comprises a CDR-H3 domain comprising or consisting of a sequence as substantially set out in SEQ ID No: 86, or a variant or fragment thereof.

Preferably, the antibody or antigen-binding fragment thereof comprises a CDR-H1 domain comprising or consisting of SEQ ID No: 84, a CDR-H2 domain comprising or consisting of SEQ ID No: 85 and/or a CDR-H3 domain comprising or consisting of SEQ ID No: 86. Preferably, however, the antibody or antigen-binding fragment thereof comprises a CDR-H1 domain comprising or consisting of SEQ ID No: 84, a CDR-H2 domain comprising or consisting of SEQ ID No: 85 and a CDR-H3 domain comprising or consisting of SEQ ID No: 86.

Preferably, the antibody or antigen-binding fragment thereof comprises a heavy chain variable (VH) region comprising or consisting of a sequence as substantially set out in SEQ ID No: 99, or a variant or fragment thereof.

Preferably, the antibody or antigen-binding fragment thereof comprises a CDR-L1 domain comprising or consisting of a sequence as substantially set out in SEQ ID No: 87, or a variant or fragment thereof. Preferably, the antibody or antigen-binding fragment thereof comprises a CDR-L2 domain comprising or consisting of a sequence as substantially set out in SEQ ID No: 88, or a variant or fragment thereof. Preferably, the antibody or antigen-binding fragment thereof comprises a CDR-L3 domain comprising or consisting of a sequence as substantially set out in SEQ ID No: 89, or a

Preferably, the antibody or antigen-binding fragment thereof comprises a CDR-L1 domain comprising or consisting of SEQ ID No: 87, a CDR-L2 domain comprising or consisting of SEQ ID No: 88, and/or a CDR-L3 domain comprising or consisting of SEQ ID No: 89. However, preferably the antibody or antigen-binding fragment thereof comprises a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a light chain variable region comprising or consisting of a sequence as substantially set out in SEQ ID No: 96, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 84, a CDR-H2 domain comprising or consisting of SEQ ID No: 85; a CDR-H3 domain comprising or consisting of SEQ ID No: 86, a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a heavy chain variable region comprising or consisting of SEQ ID No: 99, and a light chain variable region comprising or consisting of SEQ ID No: 96.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 33D03 VH-5 VL-1

Alternatively, in another embodiment, the antibody or antigen-binding fragment thereof is a humanised antibody. In one embodiment, the humanised antibody or antigen-binding fragment thereof is referred to herein as 33D03_VH-5_VL-1.

Preferably, the antibody or antigen-binding fragment thereof comprises a CDR-H1 domain comprising or consisting of a sequence as substantially set out in SEQ ID No: 84, or a variant or fragment thereof. Preferably, the antibody or antigen-binding fragment thereof comprises a CDR-H2 domain comprising or consisting of a sequence as substantially set out in SEQ ID No: 85, or a variant or fragment thereof. Preferably, the antibody or antigen-binding fragment thereof comprises a CDR-H3 domain comprising or consisting of a sequence as substantially set out in SEQ ID No: 86, or a variant or fragment thereof.

Preferably, the antibody or antigen-binding fragment thereof comprises a CDR-H1 domain comprising or consisting of SEQ ID No: 84, a CDR-H2 domain comprising or consisting of SEQ ID No: 85 and/or a CDR-H3 domain comprising or consisting of SEQ ID No: 86. Preferably, however, the antibody or antigen-binding fragment thereof comprises a CDR-H1 domain comprising or consisting of SEQ ID No: 84, a CDR-H2 domain comprising or consisting of SEQ ID No: 85 and a CDR-H3 domain comprising or consisting of SEQ ID No: 86.

Preferably, the antibody or antigen-binding fragment thereof comprises a heavy chain variable (VH) region comprising or consisting of a sequence as substantially set out in SEQ ID No: 100, or a variant or fragment thereof.

Preferably, the antibody or antigen-binding fragment thereof comprises a CDR-L1 domain comprising or consisting of a sequence as substantially set out in SEQ ID No: 87, or a variant or fragment thereof. Preferably, the antibody or antigen-binding fragment thereof comprises a CDR-L2 domain comprising or consisting of a sequence as substantially set out in SEQ ID No: 88, or a variant or fragment thereof. Preferably, the antibody or antigen-binding fragment thereof comprises a CDR-L3 domain comprising or consisting of a sequence as substantially set out in SEQ ID No: 89, or a variant or fragment thereof.

Preferably, the antibody or antigen-binding fragment thereof comprises a CDR-L1 domain comprising or consisting of SEQ ID No: 87, a CDR-L2 domain comprising or consisting of SEQ ID No: 88, and/or a CDR-L3 domain comprising or consisting of SEQ ID No: 89. However, preferably the antibody or antigen-binding fragment thereof comprises a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a light chain variable region comprising or consisting of a sequence as substantially set out in SEQ ID No: 91, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 84, a CDR-H2 domain comprising or consisting of SEQ ID No: 85; a CDR-H3 domain comprising or consisting of SEQ ID No: 86, a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a heavy chain variable region comprising or consisting of SEQ ID No: 100, and a light chain variable region comprising or consisting of SEQ ID No: 91.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 33D03 VH-5 VL-2

Alternatively, in another embodiment, the antibody or antigen-binding fragment thereof is a humanised antibody. In one embodiment, the humanised antibody or antigen-binding fragment thereof is referred to herein as 33D03_VH-5_VL-2.

Preferably, the antibody or antigen-binding fragment thereof comprises a CDR-H1 domain comprising or consisting of a sequence as substantially set out in SEQ ID No: 84, or a variant or fragment thereof. Preferably, the antibody or antigen-binding fragment thereof comprises a CDR-H2 domain comprising or consisting of a sequence as substantially set out in SEQ ID No: 85, or a variant or fragment thereof. Preferably, the antibody or antigen-binding fragment thereof comprises a CDR-H3 domain comprising or consisting of a sequence as substantially set out in SEQ ID No: 86, or a variant or fragment thereof.

Preferably, the antibody or antigen-binding fragment thereof comprises a CDR-H1 domain comprising or consisting of SEQ ID No: 84, a CDR-H2 domain comprising or consisting of SEQ ID No: 85 and/or a CDR-H3 domain comprising or consisting of SEQ ID No: 86. Preferably, however, the antibody or antigen-binding fragment thereof comprises a CDR-H1 domain comprising or consisting of SEQ ID No: 84, a CDR-H2 domain comprising or consisting of SEQ ID No: 85 and a CDR-H3 domain comprising or consisting of SEQ ID No: 86.

Preferably, the antibody or antigen-binding fragment thereof comprises a heavy chain variable (VH) region comprising or consisting of a sequence as substantially set out in SEQ ID No: 100, or a variant or fragment thereof.

Preferably, the antibody or antigen-binding fragment thereof comprises a CDR-L1 domain comprising or consisting of a sequence as substantially set out in SEQ ID No: 87, or a variant or fragment thereof. Preferably, the antibody or antigen-binding fragment thereof comprises a CDR-L2 domain comprising or consisting of a sequence as substantially set out in SEQ ID No: 88, or a variant or fragment thereof. Preferably, the antibody or antigen-binding fragment thereof comprises a CDR-L3 domain comprising or consisting of a sequence as substantially set out in SEQ ID No: 89, or a variant or fragment thereof.

Preferably, the antibody or antigen-binding fragment thereof comprises a CDR-L1 domain comprising or consisting of SEQ ID No: 87, a CDR-L2 domain comprising or consisting of SEQ ID No: 88, and/or a CDR-L3 domain comprising or consisting of SEQ ID No: 89. However, preferably the antibody or antigen-binding fragment thereof comprises a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a light chain variable region comprising or consisting of a sequence as substantially set out in SEQ ID No: 94, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 84, a CDR-H2 domain comprising or consisting of SEQ ID No: 85; a CDR-H3 domain comprising or consisting of SEQ ID No: 86, a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a heavy chain variable region comprising or consisting of SEQ ID No: 100, and a light chain variable region comprising or consisting of SEQ ID No: 94.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 33D03 VH-5 VL-3

Alternatively, in another embodiment, the antibody or antigen-binding fragment thereof is a humanised antibody. In one embodiment, the humanised antibody or antigen-binding fragment thereof is referred to herein as 33D03_VH-5_VL-3.

Preferably, the antibody or antigen-binding fragment thereof comprises a CDR-H1 domain comprising or consisting of a sequence as substantially set out in SEQ ID No: 84, or a variant or fragment thereof. Preferably, the antibody or antigen-binding fragment thereof comprises a CDR-H2 domain comprising or consisting of a sequence as substantially set out in SEQ ID No: 85, or a variant or fragment thereof. Preferably, the antibody or antigen-binding fragment thereof comprises a CDR-H3 domain comprising or consisting of a sequence as substantially set out in SEQ ID No: 86, or a variant or fragment thereof.

Preferably, the antibody or antigen-binding fragment thereof comprises a CDR-H1 domain comprising or consisting of SEQ ID No: 84, a CDR-H2 domain comprising or consisting of SEQ ID No: 85 and/or a CDR-H3 domain comprising or consisting of SEQ ID No: 86. Preferably, however, the antibody or antigen-binding fragment thereof comprises a CDR-H1 domain comprising or consisting of SEQ ID No: 84, a CDR-H2 domain comprising or consisting of SEQ ID No: 85 and a CDR-H3 domain comprising or consisting of SEQ ID No: 86.

Preferably, the antibody or antigen-binding fragment thereof comprises a heavy chain variable (VH) region comprising or consisting of a sequence as substantially set out in SEQ ID No: 100, or a variant or fragment thereof.

Preferably, the antibody or antigen-binding fragment thereof comprises a CDR-L1 domain comprising or consisting of a sequence as substantially set out in SEQ ID No: 87, or a variant or fragment thereof. Preferably, the antibody or antigen-binding fragment thereof comprises a CDR-L2 domain comprising or consisting of a sequence as substantially set out in SEQ ID No: 88, or a variant or fragment thereof. Preferably, the antibody or antigen-binding fragment thereof comprises a CDR-L3 domain comprising or consisting of a sequence as substantially set out in SEQ ID No: 89, or a variant or fragment thereof.

Preferably, the antibody or antigen-binding fragment thereof comprises a CDR-L1 domain comprising or consisting of SEQ ID No: 87, a CDR-L2 domain comprising or consisting of SEQ ID No: 88, and/or a CDR-L3 domain comprising or consisting of SEQ ID No: 89. However, preferably the antibody or antigen-binding fragment thereof comprises a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a light chain variable region comprising or consisting of a sequence as substantially set out in SEQ ID No: 95, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 84, a CDR-H2 domain comprising or consisting of SEQ ID No: 85; a CDR-H3 domain comprising or consisting of SEQ ID No: 86, a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a heavy chain variable region comprising or consisting of SEQ ID No: 100, and a light chain variable region comprising or consisting of SEQ ID No: 95.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 33D03 VH-5 VL-4

Alternatively, in another embodiment, the antibody or antigen-binding fragment thereof is a humanised antibody. In one embodiment, the humanised antibody or antigen-binding fragment thereof is referred to herein as 33D03_VH-5_VL-4.

Preferably, the antibody or antigen-binding fragment thereof comprises a CDR-H1 domain comprising or consisting of a sequence as substantially set out in SEQ ID No: 84, or a variant or fragment thereof. Preferably, the antibody or antigen-binding fragment thereof comprises a CDR-H2 domain comprising or consisting of a sequence as substantially set out in SEQ ID No: 85, or a variant or fragment thereof. Preferably, the antibody or antigen-binding fragment thereof comprises a CDR-H3 domain comprising or consisting of a sequence as substantially set out in SEQ ID No: 86, or a variant or fragment thereof.

Preferably, the antibody or antigen-binding fragment thereof comprises a CDR-H1 domain comprising or consisting of SEQ ID No: 84, a CDR-H2 domain comprising or consisting of SEQ ID No: 85 and/or a CDR-H3 domain comprising or consisting of SEQ ID No: 86. Preferably, however, the antibody or antigen-binding fragment thereof comprises a CDR-H1 domain comprising or consisting of SEQ ID No: 84, a CDR-H2 domain comprising or consisting of SEQ ID No: 85 and a CDR-H3 domain comprising or consisting of SEQ ID No: 86.

Preferably, the antibody or antigen-binding fragment thereof comprises a heavy chain variable (VH) region comprising or consisting of a sequence as substantially set out in SEQ ID No: 100, or a variant or fragment thereof.

Preferably, the antibody or antigen-binding fragment thereof comprises a CDR-L1 domain comprising or consisting of a sequence as substantially set out in SEQ ID No: 87, or a variant or fragment thereof. Preferably, the antibody or antigen-binding fragment thereof comprises a CDR-L2 domain comprising or consisting of a sequence as substantially set out in SEQ ID No: 88, or a variant or fragment thereof. Preferably, the antibody or antigen-binding fragment thereof comprises a CDR-L3 domain comprising or consisting of a sequence as substantially set out in SEQ ID No: 89, or a variant or fragment thereof.

Preferably, the antibody or antigen-binding fragment thereof comprises a CDR-L1 domain comprising or consisting of SEQ ID No: 87, a CDR-L2 domain comprising or consisting of SEQ ID No: 88, and/or a CDR-L3 domain comprising or consisting of SEQ ID No: 89. However, preferably the antibody or antigen-binding fragment thereof comprises a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a light chain variable region comprising or consisting of a sequence as substantially set out in SEQ ID No: 96, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 84, a CDR-H2 domain comprising or consisting of SEQ ID No: 85; a CDR-H3 domain comprising or consisting of SEQ ID No: 86, a CDR-L1 domain comprising or consisting of SEQ ID No: 87, a CDR-L2 domain comprising or consisting of SEQ ID No: 88, and a CDR-L3 domain comprising or consisting of SEQ ID No: 89.

Preferably, the antibody or antigen-binding fragment thereof comprises a heavy chain variable region comprising or consisting of SEQ ID No: 100, and a light chain variable region comprising or consisting of SEQ ID No: 96.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 25B05 H1 VH-1 VL-1

Alternatively, in another embodiment, the antibody or antigen-binding fragment thereof is a humanised antibody. In one embodiment, the humanised antibody or antigen-binding fragment thereof is referred to herein as 25B05_H1_VH-1_VL-1.

Preferably, the antibody or antigen-binding fragment thereof comprises a CDR-H1 domain comprising or consisting of a sequence as substantially set out in SEQ ID No: 101, or a variant or fragment thereof. Preferably, the antibody or antigen-binding fragment thereof comprises a CDR-H2 domain comprising or consisting of a sequence as substantially set out in SEQ ID No: 102, or a variant or fragment thereof. Preferably, the antibody or antigen-binding fragment thereof comprises a CDR-H3 domain comprising or consisting of a sequence as substantially set out in SEQ ID No: 103, or a variant or fragment thereof.

Preferably, the antibody or antigen-binding fragment thereof comprises a CDR-H1 domain comprising or consisting of SEQ ID No: 101, a CDR-H2 domain comprising or consisting of SEQ ID No: 102 and/or a CDR-H3 domain comprising or consisting of SEQ ID No: 103. Preferably, however, the antibody or antigen-binding fragment thereof comprises a CDR-H1 domain comprising or consisting of SEQ ID No: 101, a CDR-H2 domain comprising or consisting of SEQ ID No: 102 and a CDR-H3 domain comprising or consisting of SEQ ID No: 103.

Preferably, the antibody or antigen-binding fragment thereof comprises a heavy chain variable (VH) region comprising or consisting of a sequence as substantially set out in SEQ ID No: 107, or a variant or fragment thereof.

Preferably, the antibody or antigen-binding fragment thereof comprises a CDR-L1 domain comprising or consisting of a sequence as substantially set out in SEQ ID No: 104, or a variant or fragment thereof. Preferably, the antibody or antigen-binding fragment thereof comprises a CDR-L2 domain comprising or consisting of a sequence as substantially set out in SEQ ID No: 105, or a variant or fragment thereof. Preferably, the antibody or antigen-binding fragment thereof comprises a CDR-L3 domain comprising or consisting of a sequence as substantially set out in SEQ ID No: 106, or a variant or fragment thereof.

Preferably, the antibody or antigen-binding fragment thereof comprises a CDR-L1 domain comprising or consisting of SEQ ID No: 104, a CDR-L2 domain comprising or consisting of SEQ ID No: 105, and/or a CDR-L3 domain comprising or consisting of SEQ ID No: 106. However, preferably the antibody or antigen-binding fragment thereof comprises a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a light chain variable region comprising or consisting of a sequence as substantially set out in SEQ ID No: 108, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 101, a CDR-H2 domain comprising or consisting of SEQ ID No: 102; a CDR-H3 domain comprising or consisting of SEQ ID No: 103, a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a heavy chain variable region comprising or consisting of SEQ ID No: 107, and a light chain variable region comprising or consisting of SEQ ID No: 108.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 25B05 H1 VH-1 VL-2

Alternatively, in another embodiment, the antibody or antigen-binding fragment thereof is a humanised antibody. In one embodiment, the humanised antibody or antigen-binding fragment thereof is referred to herein as 25B05_H1_VH-1_VL-2.

Preferably, the antibody or antigen-binding fragment thereof comprises a CDR-H1 domain comprising or consisting of a sequence as substantially set out in SEQ ID No: 101, or a variant or fragment thereof. Preferably, the antibody or antigen-binding fragment thereof comprises a CDR-H2 domain comprising or consisting of a sequence as substantially set out in SEQ ID No: 102, or a variant or fragment thereof. Preferably, the antibody or antigen-binding fragment thereof comprises a CDR-H3 domain comprising or consisting of a sequence as substantially set out in SEQ ID No: 103, or a variant or fragment thereof.

Preferably, the antibody or antigen-binding fragment thereof comprises a CDR-H1 domain comprising or consisting of SEQ ID No: 101, a CDR-H2 domain comprising or consisting of SEQ ID No: 102 and/or a CDR-H3 domain comprising or consisting of SEQ ID No: 103. Preferably, however, the antibody or antigen-binding fragment thereof comprises a CDR-H1 domain comprising or consisting of SEQ ID No: 101, a CDR-H2 domain comprising or consisting of SEQ ID No: 102 and a CDR-H3 domain comprising or consisting of SEQ ID No: 103.

Preferably, the antibody or antigen-binding fragment thereof comprises a heavy chain variable (VH) region comprising or consisting of a sequence as substantially set out in SEQ ID No: 107, or a variant or fragment thereof.

Preferably, the antibody or antigen-binding fragment thereof comprises a CDR-L1 domain comprising or consisting of a sequence as substantially set out in SEQ ID No: 104, or a variant or fragment thereof. Preferably, the antibody or antigen-binding fragment thereof comprises a CDR-L2 domain comprising or consisting of a sequence as substantially set out in SEQ ID No: 105, or a variant or fragment thereof. Preferably, the antibody or antigen-binding fragment thereof comprises a CDR-L3 domain comprising or consisting of a sequence as substantially set out in SEQ ID No: 106, or a variant or fragment thereof.

Preferably, the antibody or antigen-binding fragment thereof comprises a CDR-L1 domain comprising or consisting of SEQ ID No: 104, a CDR-L2 domain comprising or consisting of SEQ ID No: 105, and/or a CDR-L3 domain comprising or consisting of SEQ ID No: 106. However, preferably the antibody or antigen-binding fragment thereof comprises a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a light chain variable region comprising or consisting of a sequence as substantially set out in SEQ ID No: 109, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 101, a CDR-H2 domain comprising or consisting of SEQ ID No: 102; a CDR-H3 domain comprising or consisting of SEQ ID No: 103, a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a heavy chain variable region comprising or consisting of SEQ ID No: 107, and a light chain variable region comprising or consisting of SEQ ID No: 109.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 25B05 H1 VH-1 VL-3

Alternatively, in another embodiment, the antibody or antigen-binding fragment thereof is a humanised antibody. In one embodiment, the humanised antibody or antigen-binding fragment thereof is referred to herein as 25B05_H1_VH-1_VL-3.

Preferably, the antibody or antigen-binding fragment thereof comprises a CDR-H1 domain comprising or consisting of a sequence as substantially set out in SEQ ID No: 101, or a variant or fragment thereof. Preferably, the antibody or antigen-binding fragment thereof comprises a CDR-H2 domain comprising or consisting of a sequence as substantially set out in SEQ ID No: 102, or a variant or fragment thereof. Preferably, the antibody or antigen-binding fragment thereof comprises a CDR-H3 domain comprising or consisting of a sequence as substantially set out in SEQ ID No: 103, or a variant or fragment thereof.

Preferably, the antibody or antigen-binding fragment thereof comprises a CDR-H1 domain comprising or consisting of SEQ ID No: 101, a CDR-H2 domain comprising or consisting of SEQ ID No: 102 and/or a CDR-H3 domain comprising or consisting of SEQ ID No: 103. Preferably, however, the antibody or antigen-binding fragment thereof comprises a CDR-H1 domain comprising or consisting of SEQ ID No: 101, a CDR-H2 domain comprising or consisting of SEQ ID No: 102 and a CDR-H3 domain comprising or consisting of SEQ ID No: 103.

Preferably, the antibody or antigen-binding fragment thereof comprises a heavy chain variable (VH) region comprising or consisting of a sequence as substantially set out in SEQ ID No: 107, or a variant or fragment thereof.

Preferably, the antibody or antigen-binding fragment thereof comprises a CDR-L1 domain comprising or consisting of a sequence as substantially set out in SEQ ID No: 104, or a variant or fragment thereof. Preferably, the antibody or antigen-binding fragment thereof comprises a CDR-L2 domain comprising or consisting of a sequence as substantially set out in SEQ ID No: 105, or a variant or fragment thereof. Preferably, the antibody or antigen-binding fragment thereof comprises a CDR-L3 domain comprising or consisting of a sequence as substantially set out in SEQ ID No: 106, or a variant or fragment thereof.

Preferably, the antibody or antigen-binding fragment thereof comprises a CDR-L1 domain comprising or consisting of SEQ ID No: 104, a CDR-L2 domain comprising or consisting of SEQ ID No: 105, and/or a CDR-L3 domain comprising or consisting of SEQ ID No: 106. However, preferably the antibody or antigen-binding fragment thereof comprises a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a light chain variable region comprising or consisting of a sequence as substantially set out in SEQ ID No: 110, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 101, a CDR-H2 domain comprising or consisting of SEQ ID No: 102; a CDR-H3 domain comprising or consisting of SEQ ID No: 103, a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a heavy chain variable region comprising or consisting of SEQ ID No: 107, and a light chain variable region comprising or consisting of SEQ ID No: 110.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 25B05 H1 VH-1 VL-4

Alternatively, in another embodiment, the antibody or antigen-binding fragment thereof is a humanised antibody. In one embodiment, the humanised antibody or antigen-binding fragment thereof is referred to herein as 25B05_H1_VH-1_VL-4.

Preferably, the antibody or antigen-binding fragment thereof comprises a CDR-H1 domain comprising or consisting of a sequence as substantially set out in SEQ ID No: 101, or a variant or fragment thereof. Preferably, the antibody or antigen-binding fragment thereof comprises a CDR-H2 domain comprising or consisting of a sequence as substantially set out in SEQ ID No: 102, or a variant or fragment thereof. Preferably, the antibody or antigen-binding fragment thereof comprises a CDR-H3 domain comprising or consisting of a sequence as substantially set out in SEQ ID No: 103, or a variant or fragment thereof.

Preferably, the antibody or antigen-binding fragment thereof comprises a CDR-H1 domain comprising or consisting of SEQ ID No: 101, a CDR-H2 domain comprising or consisting of SEQ ID No: 102 and/or a CDR-H3 domain comprising or consisting of SEQ ID No: 103. Preferably, however, the antibody or antigen-binding fragment thereof comprises a CDR-H1 domain comprising or consisting of SEQ ID No: 101, a CDR-H2 domain comprising or consisting of SEQ ID No: 102 and a CDR-H3 domain comprising or consisting of SEQ ID No: 103.

Preferably, the antibody or antigen-binding fragment thereof comprises a heavy chain variable (VH) region comprising or consisting of a sequence as substantially set out in SEQ ID No: 107, or a variant or fragment thereof.

Preferably, the antibody or antigen-binding fragment thereof comprises a CDR-L1 domain comprising or consisting of a sequence as substantially set out in SEQ ID No: 104, or a variant or fragment thereof. Preferably, the antibody or antigen-binding fragment thereof comprises a CDR-L2 domain comprising or consisting of a sequence as substantially set out in SEQ ID No: 105, or a variant or fragment thereof. Preferably, the antibody or antigen-binding fragment thereof comprises a CDR-L3 domain comprising or consisting of a sequence as substantially set out in SEQ ID No: 106, or a variant or fragment thereof.

Preferably, the antibody or antigen-binding fragment thereof comprises a CDR-L1 domain comprising or consisting of SEQ ID No: 104, a CDR-L2 domain comprising or consisting of SEQ ID No: 105, and/or a CDR-L3 domain comprising or consisting of SEQ ID No: 106. However, preferably the antibody or antigen-binding fragment thereof comprises a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a light chain variable region comprising or consisting of a sequence as substantially set out in SEQ ID No: 111, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 101, a CDR-H2 domain comprising or consisting of SEQ ID No: 102; a CDR-H3 domain comprising or consisting of SEQ ID No: 103, a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a heavy chain variable region comprising or consisting of SEQ ID No: 107, and a light chain variable region comprising or consisting of SEQ ID No: 111.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 25B05 H1 VH-2 VL-1

Alternatively, in another embodiment, the antibody or antigen-binding fragment thereof is a humanised antibody. In one embodiment, the humanised antibody or antigen-binding fragment thereof is referred to herein as 25B05_H1_VH-2_VL-1.

Preferably, the antibody or antigen-binding fragment thereof comprises a CDR-H1 domain comprising or consisting of a sequence as substantially set out in SEQ ID No: 101, or a variant or fragment thereof. Preferably, the antibody or antigen-binding fragment thereof comprises a CDR-H2 domain comprising or consisting of a sequence as substantially set out in SEQ ID No: 102, or a variant or fragment thereof. Preferably, the antibody or antigen-binding fragment thereof comprises a CDR-H3 domain comprising or consisting of a sequence as substantially set out in SEQ ID No: 103, or a variant or fragment thereof.

Preferably, the antibody or antigen-binding fragment thereof comprises a CDR-H1 domain comprising or consisting of SEQ ID No: 101, a CDR-H2 domain comprising or consisting of SEQ ID No: 102 and/or a CDR-H3 domain comprising or consisting of SEQ ID No: 103. Preferably, however, the antibody or antigen-binding fragment thereof comprises a CDR-H1 domain comprising or consisting of SEQ ID No: 101, a CDR-H2 domain comprising or consisting of SEQ ID No: 102 and a CDR-H3 domain comprising or consisting of SEQ ID No: 103.

Preferably, the antibody or antigen-binding fragment thereof comprises a heavy chain variable (VH) region comprising or consisting of a sequence as substantially set out in SEQ ID No: 112, or a variant or fragment thereof.

Preferably, the antibody or antigen-binding fragment thereof comprises a CDR-L1 domain comprising or consisting of a sequence as substantially set out in SEQ ID No: 104, or a variant or fragment thereof. Preferably, the antibody or antigen-binding fragment thereof comprises a CDR-L2 domain comprising or consisting of a sequence as substantially set out in SEQ ID No: 105, or a variant or fragment thereof. Preferably, the antibody or antigen-binding fragment thereof comprises a CDR-L3 domain comprising or consisting of a sequence as substantially set out in SEQ ID No: 106, or a variant or fragment thereof.

Preferably, the antibody or antigen-binding fragment thereof comprises a CDR-L1 domain comprising or consisting of SEQ ID No: 104, a CDR-L2 domain comprising or consisting of SEQ ID No: 105, and/or a CDR-L3 domain comprising or consisting of SEQ ID No: 106. However, preferably the antibody or antigen-binding fragment thereof comprises a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a light chain variable region comprising or consisting of a sequence as substantially set out in SEQ ID No: 108, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 101, a CDR-H2 domain comprising or consisting of SEQ ID No: 102; a CDR-H3 domain comprising or consisting of SEQ ID No: 103, a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a heavy chain variable region comprising or consisting of SEQ ID No: 112, and a light chain variable region comprising or consisting of SEQ ID No: 108.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 25B05 H1 VH-2 VL-2

Alternatively, in another embodiment, the antibody or antigen-binding fragment thereof is humanised antibody. In one embodiment, the humanised antibody or antigen-binding fragment thereof is referred to herein as 25B05_H1_VH-2_VL-2.

Preferably, the antibody or antigen-binding fragment thereof comprises a CDR-H1 domain comprising or consisting of a sequence as substantially set out in SEQ ID No: 101, or a variant or fragment thereof. Preferably, the antibody or antigen-binding fragment thereof comprises a CDR-H2 domain comprising or consisting of a sequence as substantially set out in SEQ ID No: 102, or a variant or fragment thereof. Preferably, the antibody or antigen-binding fragment thereof comprises a CDR-H3 domain comprising or consisting of a sequence as substantially set out in SEQ ID No: 103, or a variant or fragment thereof.

Preferably, the antibody or antigen-binding fragment thereof comprises a CDR-H1 domain comprising or consisting of SEQ ID No: 101, a CDR-H2 domain comprising or consisting of SEQ ID No: 102 and/or a CDR-H3 domain comprising or consisting of SEQ ID No: 103. Preferably, however, the antibody or antigen-binding fragment thereof comprises a CDR-H1 domain comprising or consisting of SEQ ID No: 101, a CDR-H2 domain comprising or consisting of SEQ ID No: 102 and a CDR-H3 domain comprising or consisting of SEQ ID No: 103.

Preferably, the antibody or antigen-binding fragment thereof comprises a heavy chain variable (VH) region comprising or consisting of a sequence as substantially set out in SEQ ID No: 112, or a variant or fragment thereof.

Preferably, the antibody or antigen-binding fragment thereof comprises a CDR-L1 domain comprising or consisting of a sequence as substantially set out in SEQ ID No: 104, or a variant or fragment thereof. Preferably, the antibody or antigen-binding fragment thereof comprises a CDR-L2 domain comprising or consisting of a sequence as substantially set out in SEQ ID No: 105, or a variant or fragment thereof. Preferably, the antibody or antigen-binding fragment thereof comprises a CDR-L3 domain comprising or consisting of a sequence as substantially set out in SEQ ID No: 106, or a variant or fragment thereof.

Preferably, the antibody or antigen-binding fragment thereof comprises a CDR-L1 domain comprising or consisting of SEQ ID No: 104, a CDR-L2 domain comprising or consisting of SEQ ID No: 105, and/or a CDR-L3 domain comprising or consisting of SEQ ID No: 106. However, preferably the antibody or antigen-binding fragment thereof comprises a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a light chain variable region comprising or consisting of a sequence as substantially set out in SEQ ID No: 109, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 101, a CDR-H2 domain comprising or consisting of SEQ ID No: 102; a CDR-H3 domain comprising or consisting of SEQ ID No: 103, a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a heavy chain variable region comprising or consisting of SEQ ID No: 112, and a light chain variable region comprising or consisting of SEQ ID No: 109.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 25B05 H1 VH-2 VL-3

Alternatively, in another embodiment, the antibody or antigen-binding fragment thereof is a humanised antibody. In one embodiment, the humanised antibody or antigen-binding fragment thereof is referred to herein as 25B05_H1_VH-2_VL-3.

Preferably, the antibody or antigen-binding fragment thereof comprises a CDR-H1 domain comprising or consisting of a sequence as substantially set out in SEQ ID No: 101, or a variant or fragment thereof. Preferably, the antibody or antigen-binding fragment thereof comprises a CDR-H2 domain comprising or consisting of a sequence as substantially set out in SEQ ID No: 102, or a variant or fragment thereof. Preferably, the antibody or antigen-binding fragment thereof comprises a CDR-H3 domain comprising or consisting of a sequence as substantially set out in SEQ ID No: 103, or a variant or fragment thereof.

Preferably, the antibody or antigen-binding fragment thereof comprises a CDR-H1 domain comprising or consisting of SEQ ID No: 101, a CDR-H2 domain comprising or consisting of SEQ ID No: 102 and/or a CDR-H3 domain comprising or consisting of SEQ ID No: 103. Preferably, however, the antibody or antigen-binding fragment thereof comprises a CDR-H1 domain comprising or consisting of SEQ ID No: 101, a CDR-H2 domain comprising or consisting of SEQ ID No: 102 and a CDR-H3 domain comprising or consisting of SEQ ID No: 103.

Preferably, the antibody or antigen-binding fragment thereof comprises a heavy chain variable (VH) region comprising or consisting of a sequence as substantially set out in SEQ ID No: 112, or a variant or fragment thereof.

Preferably, the antibody or antigen-binding fragment thereof comprises a CDR-L1 domain comprising or consisting of a sequence as substantially set out in SEQ ID No: 104, or a variant or fragment thereof. Preferably, the antibody or antigen-binding fragment thereof comprises a CDR-L2 domain comprising or consisting of a sequence as substantially set out in SEQ ID No: 105, or a variant or fragment thereof. Preferably, the antibody or antigen-binding fragment thereof comprises a CDR-L3 domain comprising or consisting of a sequence as substantially set out in SEQ ID No: 106, or a

Preferably, the antibody or antigen-binding fragment thereof comprises a CDR-L1 domain comprising or consisting of SEQ ID No: 104, a CDR-L2 domain comprising or consisting of SEQ ID No: 105, and/or a CDR-L3 domain comprising or consisting of SEQ ID No: 106. However, preferably the antibody or antigen-binding fragment thereof comprises a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a light chain variable region comprising or consisting of a sequence as substantially set out in SEQ ID No: 110, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 101, a CDR-H2 domain comprising or consisting of SEQ ID No: 102; a CDR-H3 domain comprising or consisting of SEQ ID No: 103, a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a heavy chain variable region comprising or consisting of SEQ ID No: 112, and a light chain variable region comprising or consisting of SEQ ID No: 110.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 25B05 H1 VH-2 VL-4

Alternatively, in another embodiment, the antibody or antigen-binding fragment thereof is a humanised antibody. In one embodiment, the humanised antibody or antigen-binding fragment thereof is referred to herein as 25B05_H1_VH-2_VL-4.

Preferably, the antibody or antigen-binding fragment thereof comprises a CDR-H1 domain comprising or consisting of a sequence as substantially set out in SEQ ID No: 101, or a variant or fragment thereof. Preferably, the antibody or antigen-binding fragment thereof comprises a CDR-H2 domain comprising or consisting of a sequence as substantially set out in SEQ ID No: 102, or a variant or fragment thereof. Preferably, the antibody or antigen-binding fragment thereof comprises a CDR-H3 domain comprising or consisting of a sequence as substantially set out in SEQ ID No: 103, or a variant or fragment thereof.

Preferably, the antibody or antigen-binding fragment thereof comprises a CDR-H1 domain comprising or consisting of SEQ ID No: 101, a CDR-H2 domain comprising or consisting of SEQ ID No: 102 and/or a CDR-H3 domain comprising or consisting of SEQ ID No: 103. Preferably, however, the antibody or antigen-binding fragment thereof comprises a CDR-H1 domain comprising or consisting of SEQ ID No: 101, a CDR-H2 domain comprising or consisting of SEQ ID No: 102 and a CDR-H3 domain comprising or consisting of SEQ ID No: 103.

Preferably, the antibody or antigen-binding fragment thereof comprises a heavy chain variable (VH) region comprising or consisting of a sequence as substantially set out in SEQ ID No: 112, or a variant or fragment thereof.

Preferably, the antibody or antigen-binding fragment thereof comprises a CDR-L1 domain comprising or consisting of a sequence as substantially set out in SEQ ID No: 104, or a variant or fragment thereof. Preferably, the antibody or antigen-binding fragment thereof comprises a CDR-L2 domain comprising or consisting of a sequence as substantially set out in SEQ ID No: 105, or a variant or fragment thereof. Preferably, the antibody or antigen-binding fragment thereof comprises a CDR-L3 domain comprising or consisting of a sequence as substantially set out in SEQ ID No: 106, or a variant or fragment thereof.

Preferably, the antibody or antigen-binding fragment thereof comprises a CDR-L1 domain comprising or consisting of SEQ ID No: 104, a CDR-L2 domain comprising or consisting of SEQ ID No: 105, and/or a CDR-L3 domain comprising or consisting of SEQ ID No: 106. However, preferably the antibody or antigen-binding fragment thereof comprises a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a light chain variable region comprising or consisting of a sequence as substantially set out in SEQ ID No: 111, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 101, a CDR-H2 domain comprising or consisting of SEQ ID No: 102; a CDR-H3 domain comprising or consisting of SEQ ID No: 103, a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a heavy chain variable region comprising or consisting of SEQ ID No: 112, and a light chain variable region comprising or consisting of SEQ ID No: 111.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 25B05 H1 VH-3 VL-1

Alternatively, in another embodiment, the antibody or antigen-binding fragment thereof is a humanised antibody. In one embodiment, the humanised antibody or antigen-binding fragment thereof is referred to herein as 25B05_H1_VH-3_VL-1.

Preferably, the antibody or antigen-binding fragment thereof comprises a CDR-H1 domain comprising or consisting of a sequence as substantially set out in SEQ ID No: 101, or a variant or fragment thereof. Preferably, the antibody or antigen-binding fragment thereof comprises a CDR-H2 domain comprising or consisting of a sequence as substantially set out in SEQ ID No: 102, or a variant or fragment thereof. Preferably, the antibody or antigen-binding fragment thereof comprises a CDR-H3 domain comprising or consisting of a sequence as substantially set out in SEQ ID No: 103, or a variant or fragment thereof.

Preferably, the antibody or antigen-binding fragment thereof comprises a CDR-H1 domain comprising or consisting of SEQ ID No: 101, a CDR-H2 domain comprising or consisting of SEQ ID No: 102 and/or a CDR-H3 domain comprising or consisting of SEQ ID No: 103. Preferably, however, the antibody or antigen-binding fragment thereof comprises a CDR-H1 domain comprising or consisting of SEQ ID No: 101, a CDR-H2 domain comprising or consisting of SEQ ID No: 102 and a CDR-H3 domain comprising or consisting of SEQ ID No: 103.

Preferably, the antibody or antigen-binding fragment thereof comprises a heavy chain variable (VH) region comprising or consisting of a sequence as substantially set out in SEQ ID No: 113, or a variant or fragment thereof.

Preferably, the antibody or antigen-binding fragment thereof comprises a CDR-L1 domain comprising or consisting of a sequence as substantially set out in SEQ ID No: 104, or a variant or fragment thereof. Preferably, the antibody or antigen-binding fragment thereof comprises a CDR-L2 domain comprising or consisting of a sequence as substantially set out in SEQ ID No: 105, or a variant or fragment thereof. Preferably, the antibody or antigen-binding fragment thereof comprises a CDR-L3 domain comprising or consisting of a sequence as substantially set out in SEQ ID No: 106, or a variant or fragment thereof.

Preferably, the antibody or antigen-binding fragment thereof comprises a CDR-L1 domain comprising or consisting of SEQ ID No: 104, a CDR-L2 domain comprising or consisting of SEQ ID No: 105, and/or a CDR-L3 domain comprising or consisting of SEQ ID No: 106. However, preferably the antibody or antigen-binding fragment thereof comprises a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a light chain variable region comprising or consisting of a sequence as substantially set out in SEQ ID No: 108, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 101, a CDR-H2 domain comprising or consisting of SEQ ID No: 102; a CDR-H3 domain comprising or consisting of SEQ ID No: 103, a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a heavy chain variable region comprising or consisting of SEQ ID No: 113, and a light chain variable region comprising or consisting of SEQ ID No: 108.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 25B05 H1 VH-3 VL-2

Alternatively, in another embodiment, the antibody or antigen-binding fragment thereof is a humanised antibody. In one embodiment, the humanised antibody or antigen-binding fragment thereof is referred to herein as 25B05_H1_VH-3_VL-2.

Preferably, the antibody or antigen-binding fragment thereof comprises a CDR-H1 domain comprising or consisting of a sequence as substantially set out in SEQ ID No: 101, or a variant or fragment thereof. Preferably, the antibody or antigen-binding fragment thereof comprises a CDR-H2 domain comprising or consisting of a sequence as substantially set out in SEQ ID No: 102, or a variant or fragment thereof. Preferably, the antibody or antigen-binding fragment thereof comprises a CDR-H3 domain comprising or consisting of a sequence as substantially set out in SEQ ID No: 103, or a variant or fragment thereof.

Preferably, the antibody or antigen-binding fragment thereof comprises a CDR-H1 domain comprising or consisting of SEQ ID No: 101, a CDR-H2 domain comprising or consisting of SEQ ID No: 102 and/or a CDR-H3 domain comprising or consisting of SEQ ID No: 103. Preferably, however, the antibody or antigen-binding fragment thereof comprises a CDR-H1 domain comprising or consisting of SEQ ID No: 101, a CDR-H2 domain comprising or consisting of SEQ ID No: 102 and a CDR-H3 domain comprising or consisting of SEQ ID No: 103.

Preferably, the antibody or antigen-binding fragment thereof comprises a heavy chain variable (VH) region comprising or consisting of a sequence as substantially set out in SEQ ID No: 113, or a variant or fragment thereof.

Preferably, the antibody or antigen-binding fragment thereof comprises a CDR-L1 domain comprising or consisting of a sequence as substantially set out in SEQ ID No: 104, or a variant or fragment thereof. Preferably, the antibody or antigen-binding fragment thereof comprises a CDR-L2 domain comprising or consisting of a sequence as substantially set out in SEQ ID No: 105, or a variant or fragment thereof. Preferably, the antibody or antigen-binding fragment thereof comprises a CDR-L3 domain comprising or consisting of a sequence as substantially set out in SEQ ID No: 106, or a variant or fragment thereof.

Preferably, the antibody or antigen-binding fragment thereof comprises a CDR-L1 domain comprising or consisting of SEQ ID No: 104, a CDR-L2 domain comprising or consisting of SEQ ID No: 105, and/or a CDR-L3 domain comprising or consisting of SEQ ID No: 106. However, preferably the antibody or antigen-binding fragment thereof comprises a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a light chain variable region comprising or consisting of a sequence as substantially set out in SEQ ID No: 109, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 101, a CDR-H2 domain comprising or consisting of SEQ ID No: 102; a CDR-H3 domain comprising or consisting of SEQ ID No: 103, a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a heavy chain variable region comprising or consisting of SEQ ID No: 113, and a light chain variable region comprising or consisting of SEQ ID No: 109.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 25B05 H1 VH-3 VL-3

Alternatively, in another embodiment, the antibody or antigen-binding fragment thereof is a humanised antibody. In one embodiment, the humanised antibody or antigen-binding fragment thereof is referred to herein as 25B05_H1_VH-3_VL-3.

Preferably, the antibody or antigen-binding fragment thereof comprises a CDR-H1 domain comprising or consisting of a sequence as substantially set out in SEQ ID No: 101, or a variant or fragment thereof. Preferably, the antibody or antigen-binding fragment thereof comprises a CDR-H2 domain comprising or consisting of a sequence as substantially set out in SEQ ID No: 102, or a variant or fragment thereof. Preferably, the antibody or antigen-binding fragment thereof comprises a CDR-H3 domain comprising or consisting of a sequence as substantially set out in SEQ ID No: 103, or a variant or fragment thereof.

Preferably, the antibody or antigen-binding fragment thereof comprises a CDR-H1 domain comprising or consisting of SEQ ID No: 101, a CDR-H2 domain comprising or consisting of SEQ ID No: 102 and/or a CDR-H3 domain comprising or consisting of SEQ ID No: 103. Preferably, however, the antibody or antigen-binding fragment thereof comprises a CDR-H1 domain comprising or consisting of SEQ ID No: 101, a CDR-H2 domain comprising or consisting of SEQ ID No: 102 and a CDR-H3 domain comprising or consisting of SEQ ID No: 103.

Preferably, the antibody or antigen-binding fragment thereof comprises a heavy chain variable (VH) region comprising or consisting of a sequence as substantially set out in SEQ ID No: 113, or a variant or fragment thereof.

Preferably, the antibody or antigen-binding fragment thereof comprises a CDR-L1 domain comprising or consisting of a sequence as substantially set out in SEQ ID No: 104, or a variant or fragment thereof. Preferably, the antibody or antigen-binding fragment thereof comprises a CDR-L2 domain comprising or consisting of a sequence as substantially set out in SEQ ID No: 105, or a variant or fragment thereof. Preferably, the antibody or antigen-binding fragment thereof comprises a CDR-L3 domain comprising or consisting of a sequence as substantially set out in SEQ ID No: 106, or a variant or fragment thereof.

Preferably, the antibody or antigen-binding fragment thereof comprises a CDR-L1 domain comprising or consisting of SEQ ID No: 104, a CDR-L2 domain comprising or consisting of SEQ ID No: 105, and/or a CDR-L3 domain comprising or consisting of SEQ ID No: 106. However, preferably the antibody or antigen-binding fragment thereof comprises a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a light chain variable region comprising or consisting of a sequence as substantially set out in SEQ ID No: 110, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 101, a CDR-H2 domain comprising or consisting of SEQ ID No: 102; a CDR-H3 domain comprising or consisting of SEQ ID No: 103, a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a heavy chain variable region comprising or consisting of SEQ ID No: 113, and a light chain variable region comprising or consisting of SEQ ID No: 110.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 25B05 H1 VH-3 VL-4

Alternatively, in another embodiment, the antibody or antigen-binding fragment thereof is a humanised antibody. In one embodiment, the humanised antibody or antigen-binding fragment thereof is referred to herein as 25B05_H1_VH-3_VL-4.

Preferably, the antibody or antigen-binding fragment thereof comprises a CDR-H1 domain comprising or consisting of a sequence as substantially set out in SEQ ID No: 101, or a variant or fragment thereof. Preferably, the antibody or antigen-binding fragment thereof comprises a CDR-H2 domain comprising or consisting of a sequence as substantially set out in SEQ ID No: 102, or a variant or fragment thereof. Preferably, the antibody or antigen-binding fragment thereof comprises a CDR-H3 domain comprising or consisting of a sequence as substantially set out in SEQ ID No: 103, or a variant or fragment thereof.

Preferably, the antibody or antigen-binding fragment thereof comprises a CDR-H1 domain comprising or consisting of SEQ ID No: 101, a CDR-H2 domain comprising or consisting of SEQ ID No: 102 and/or a CDR-H3 domain comprising or consisting of SEQ ID No: 103. Preferably, however, the antibody or antigen-binding fragment thereof comprises a CDR-H1 domain comprising or consisting of SEQ ID No: 101, a CDR-H2 domain comprising or consisting of SEQ ID No: 102 and a CDR-H3 domain comprising or consisting of SEQ ID No: 103.

Preferably, the antibody or antigen-binding fragment thereof comprises a heavy chain variable (VH) region comprising or consisting of a sequence as substantially set out in SEQ ID No: 113, or a variant or fragment thereof.

Preferably, the antibody or antigen-binding fragment thereof comprises a CDR-L1 domain comprising or consisting of a sequence as substantially set out in SEQ ID No: 104, or a variant or fragment thereof. Preferably, the antibody or antigen-binding fragment thereof comprises a CDR-L2 domain comprising or consisting of a sequence as substantially set out in SEQ ID No: 105, or a variant or fragment thereof. Preferably, the antibody or antigen-binding fragment thereof comprises a CDR-L3 domain comprising or consisting of a sequence as substantially set out in SEQ ID No: 106, or a variant or fragment thereof.

Preferably, the antibody or antigen-binding fragment thereof comprises a CDR-L1 domain comprising or consisting of SEQ ID No: 104, a CDR-L2 domain comprising or consisting of SEQ ID No: 105, and/or a CDR-L3 domain comprising or consisting of SEQ ID No: 106. However, preferably the antibody or antigen-binding fragment thereof comprises a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a light chain variable region comprising or consisting of a sequence as substantially set out in SEQ ID No: 111, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 101, a CDR-H2 domain comprising or consisting of SEQ ID No: 102; a CDR-H3 domain comprising or consisting of SEQ ID No: 103, a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a heavy chain variable region comprising or consisting of SEQ ID No: 113, and a light chain variable region comprising or consisting of SEQ ID No: 111.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 25B05 H1 VH-4 VL-1

Alternatively, in another embodiment, the antibody or antigen-binding fragment thereof is a humanised antibody. In one embodiment, the humanised antibody or antigen-binding fragment thereof is referred to herein as 25B05_H1_VH-4_VL-1.

Preferably, the antibody or antigen-binding fragment thereof comprises a CDR-H1 domain comprising or consisting of a sequence as substantially set out in SEQ ID No: 101, or a variant or fragment thereof. Preferably, the antibody or antigen-binding fragment thereof comprises a CDR-H2 domain comprising or consisting of a sequence as substantially set out in SEQ ID No: 102, or a variant or fragment thereof. Preferably, the antibody or antigen-binding fragment thereof comprises a CDR-H3 domain comprising or consisting of a sequence as substantially set out in SEQ ID No: 103, or a variant or fragment thereof.

Preferably, the antibody or antigen-binding fragment thereof comprises a CDR-H1 domain comprising or consisting of SEQ ID No: 101, a CDR-H2 domain comprising or consisting of SEQ ID No: 102 and/or a CDR-H3 domain comprising or consisting of SEQ ID No: 103. Preferably, however, the antibody or antigen-binding fragment thereof comprises a CDR-H1 domain comprising or consisting of SEQ ID No: 101, a CDR-H2 domain comprising or consisting of SEQ ID No: 102 and a CDR-H3 domain comprising or consisting of SEQ ID No: 103.

Preferably, the antibody or antigen-binding fragment thereof comprises a heavy chain variable (VH) region comprising or consisting of a sequence as substantially set out in SEQ ID No: 114, or a variant or fragment thereof.

Preferably, the antibody or antigen-binding fragment thereof comprises a CDR-L1 domain comprising or consisting of a sequence as substantially set out in SEQ ID No: 104, or a variant or fragment thereof. Preferably, the antibody or antigen-binding fragment thereof comprises a CDR-L2 domain comprising or consisting of a sequence as substantially set out in SEQ ID No: 105, or a variant or fragment thereof. Preferably, the antibody or antigen-binding fragment thereof comprises a CDR-L3 domain comprising or consisting of a sequence as substantially set out in SEQ ID No: 106, or a variant or fragment thereof.

Preferably, the antibody or antigen-binding fragment thereof comprises a CDR-L1 domain comprising or consisting of SEQ ID No: 104, a CDR-L2 domain comprising or consisting of SEQ ID No: 105, and/or a CDR-L3 domain comprising or consisting of SEQ ID No: 106. However, preferably the antibody or antigen-binding fragment thereof comprises a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a light chain variable region comprising or consisting of a sequence as substantially set out in SEQ ID No: 108, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 101, a CDR-H2 domain comprising or consisting of SEQ ID No: 102; a CDR-H3 domain comprising or consisting of SEQ ID No: 103, a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a heavy chain variable region comprising or consisting of SEQ ID No: 114, and a light chain variable region comprising or consisting of SEQ ID No: 108.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 25B05 H1 VH-4 VL-2

Alternatively, in another embodiment, the antibody or antigen-binding fragment thereof is a humanised antibody. In one embodiment, the humanised antibody or antigen-binding fragment thereof is referred to herein as 25B05_H1_VH-4_VL-2.

Preferably, the antibody or antigen-binding fragment thereof comprises a CDR-H1 domain comprising or consisting of a sequence as substantially set out in SEQ ID No: 101, or a variant or fragment thereof. Preferably, the antibody or antigen-binding fragment thereof comprises a CDR-H2 domain comprising or consisting of a sequence as substantially set out in SEQ ID No: 102, or a variant or fragment thereof. Preferably, the antibody or antigen-binding fragment thereof comprises a CDR-H3 domain comprising or consisting of a sequence as substantially set out in SEQ ID No: 103, or a variant or fragment thereof.

Preferably, the antibody or antigen-binding fragment thereof comprises a CDR-H1 domain comprising or consisting of SEQ ID No: 101, a CDR-H2 domain comprising or consisting of SEQ ID No: 102 and/or a CDR-H3 domain comprising or consisting of SEQ ID No: 103. Preferably, however, the antibody or antigen-binding fragment thereof comprises a CDR-H1 domain comprising or consisting of SEQ ID No: 101, a CDR-H2 domain comprising or consisting of SEQ ID No: 102 and a CDR-H3 domain comprising or consisting of SEQ ID No: 103.

Preferably, the antibody or antigen-binding fragment thereof comprises a heavy chain variable (VH) region comprising or consisting of a sequence as substantially set out in SEQ ID No: 114, or a variant or fragment thereof.

Preferably, the antibody or antigen-binding fragment thereof comprises a CDR-L1 domain comprising or consisting of a sequence as substantially set out in SEQ ID No: 104, or a variant or fragment thereof. Preferably, the antibody or antigen-binding fragment thereof comprises a CDR-L2 domain comprising or consisting of a sequence as substantially set out in SEQ ID No: 105, or a variant or fragment thereof. Preferably, the antibody or antigen-binding fragment thereof comprises a CDR-L3 domain comprising or consisting of a sequence as substantially set out in SEQ ID No: 106, or a variant or fragment thereof.

Preferably, the antibody or antigen-binding fragment thereof comprises a CDR-L1 domain comprising or consisting of SEQ ID No: 104, a CDR-L2 domain comprising or consisting of SEQ ID No: 105, and/or a CDR-L3 domain comprising or consisting of SEQ ID No: 106. However, preferably the antibody or antigen-binding fragment thereof comprises a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a light chain variable region comprising or consisting of a sequence as substantially set out in SEQ ID No: 109, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 101, a CDR-H2 domain comprising or consisting of SEQ ID No: 102; a CDR-H3 domain comprising or consisting of SEQ ID No: 103, a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a heavy chain variable region comprising or consisting of SEQ ID No: 114, and a light chain variable region comprising or consisting of SEQ ID No: 109.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 25B05 H1 VH-4 VL-3

Alternatively, in another embodiment, the antibody or antigen-binding fragment thereof is a humanised antibody. In one embodiment, the humanised antibody or antigen-binding fragment thereof is referred to herein as 25B05_H1_VH-4_VL-3.

Preferably, the antibody or antigen-binding fragment thereof comprises a CDR-H1 domain comprising or consisting of a sequence as substantially set out in SEQ ID No: 101, or a variant or fragment thereof. Preferably, the antibody or antigen-binding fragment thereof comprises a CDR-H2 domain comprising or consisting of a sequence as substantially set out in SEQ ID No: 102, or a variant or fragment thereof. Preferably, the antibody or antigen-binding fragment thereof comprises a CDR-H3 domain comprising or consisting of a sequence as substantially set out in SEQ ID No: 103, or a variant or fragment thereof.

Preferably, the antibody or antigen-binding fragment thereof comprises a CDR-H1 domain comprising or consisting of SEQ ID No: 101, a CDR-H2 domain comprising or consisting of SEQ ID No: 102 and/or a CDR-H3 domain comprising or consisting of SEQ ID No: 103. Preferably, however, the antibody or antigen-binding fragment thereof comprises a CDR-H1 domain comprising or consisting of SEQ ID No: 101, a CDR-H2 domain comprising or consisting of SEQ ID No: 102 and a CDR-H3 domain comprising or consisting of SEQ ID No: 103.

Preferably, the antibody or antigen-binding fragment thereof comprises a heavy chain variable (VH) region comprising or consisting of a sequence as substantially set out in SEQ ID No: 114, or a variant or fragment thereof.

Preferably, the antibody or antigen-binding fragment thereof comprises a CDR-L1 domain comprising or consisting of a sequence as substantially set out in SEQ ID No: 104, or a variant or fragment thereof. Preferably, the antibody or antigen-binding fragment thereof comprises a CDR-L2 domain comprising or consisting of a sequence as substantially set out in SEQ ID No: 105, or a variant or fragment thereof. Preferably, the antibody or antigen-binding fragment thereof comprises a CDR-L3 domain comprising or consisting of a sequence as substantially set out in SEQ ID No: 106, or a variant or fragment thereof.

Preferably, the antibody or antigen-binding fragment thereof comprises a CDR-L1 domain comprising or consisting of SEQ ID No: 104, a CDR-L2 domain comprising or consisting of SEQ ID No: 105, and/or a CDR-L3 domain comprising or consisting of SEQ ID No: 106. However, preferably the antibody or antigen-binding fragment thereof comprises a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a light chain variable region comprising or consisting of a sequence as substantially set out in SEQ ID No: 110, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 101, a CDR-H2 domain comprising or consisting of SEQ ID No: 102; a CDR-H3 domain comprising or consisting of SEQ ID No: 103, a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a heavy chain variable region comprising or consisting of SEQ ID No: 114, and a light chain variable region comprising or consisting of SEQ ID No: 110.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

### 25B05 H1 VH-4 VL-4

Alternatively, in another embodiment, the antibody or antigen-binding fragment thereof is a humanised antibody. In one embodiment, the humanised antibody or antigen-binding fragment thereof is referred to herein as 25B05_H1_VH-4_VL-4.

Preferably, the antibody or antigen-binding fragment thereof comprises a CDR-H1 domain comprising or consisting of a sequence as substantially set out in SEQ ID No: 101, or a variant or fragment thereof. Preferably, the antibody or antigen-binding fragment thereof comprises a CDR-H2 domain comprising or consisting of a sequence as substantially set out in SEQ ID No: 102, or a variant or fragment thereof. Preferably, the antibody or antigen-binding fragment thereof comprises a CDR-H3 domain comprising or consisting of a sequence as substantially set out in SEQ ID No: 103, or a variant or fragment thereof.

Preferably, the antibody or antigen-binding fragment thereof comprises a CDR-H1 domain comprising or consisting of SEQ ID No: 101, a CDR-H2 domain comprising or consisting of SEQ ID No: 102 and/or a CDR-H3 domain comprising or consisting of SEQ ID No: 103. Preferably, however, the antibody or antigen-binding fragment thereof comprises a CDR-H1 domain comprising or consisting of SEQ ID No: 101, a CDR-H2 domain comprising or consisting of SEQ ID No: 102 and a CDR-H3 domain comprising or consisting of SEQ ID No: 103.

Preferably, the antibody or antigen-binding fragment thereof comprises a heavy chain variable (VH) region comprising or consisting of a sequence as substantially set out in SEQ ID No: 114, or a variant or fragment thereof.

Preferably, the antibody or antigen-binding fragment thereof comprises a CDR-L1 domain comprising or consisting of a sequence as substantially set out in SEQ ID No: 104, or a variant or fragment thereof. Preferably, the antibody or antigen-binding fragment thereof comprises a CDR-L2 domain comprising or consisting of a sequence as substantially set out in SEQ ID No: 105, or a variant or fragment thereof. Preferably, the antibody or antigen-binding fragment thereof comprises a CDR-L3 domain comprising or consisting of a sequence as substantially set out in SEQ ID No: 106, or a variant or fragment thereof.

Preferably, the antibody or antigen-binding fragment thereof comprises a CDR-L1 domain comprising or consisting of SEQ ID No: 104, a CDR-L2 domain comprising or consisting of SEQ ID No: 105, and/or a CDR-L3 domain comprising or consisting of SEQ ID No: 106. However, preferably the antibody or antigen-binding fragment thereof comprises a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a light chain variable region comprising or consisting of a sequence as substantially set out in SEQ ID No: 111, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 101, a CDR-H2 domain comprising or consisting of SEQ ID No: 102; a CDR-H3 domain comprising or consisting of SEQ ID No: 103, a CDR-L1 domain comprising or consisting of SEQ ID No: 104, a CDR-L2 domain comprising or consisting of SEQ ID No: 105, and a CDR-L3 domain comprising or consisting of SEQ ID No: 106.

Preferably, the antibody or antigen-binding fragment thereof comprises a heavy chain variable region comprising or consisting of SEQ ID No: 114, and a light chain variable region comprising or consisting of SEQ ID No: 111.

Preferably, the antibody or antigen-binding fragment thereof comprises a heavy chain constant (HC) region comprising or consisting of a sequence as substantially set out in SEQ ID No: 92, or a variant or fragment thereof.

Preferably, the antibody or antigen-binding fragment thereof comprises a light chain constant (LC) region comprising or consisting of a sequence as substantially set out in SEQ ID No: 93, or a variant or fragment thereof.

Preferably, the antibody or antigen-binding fragment thereof comprises a heavy chain constant region comprising or consisting of SEQ ID No: 92 and a light chain constant region comprising or consisting of SEQ ID No: 93.

The Fc fragment is involved in platelet aggregation, and therefore, is associated with an increased risk of blood clotting and thrombosis. As such, it is particularly advantageous to disable the Fc fragment of the antibody and reduce the risk of blood clotting, when treating a patient with a condition caused by platelet-mediated aggregation.

Thus, in one embodiment, the antibody or antigen-binding fragment thereof of the invention comprises a disabled Fc fragment. Preferably, the disabled Fc fragment comprises one or more amino acid substitution that silences or reduces the effector function of the antibody or antigen-binding fragment thereof. Preferably, the disabled Fc fragment comprises one or more amino acid substitution selected from the group consisting of: L234A, L235A, and P329G. Preferably, the disabled Fc fragment comprises the amino acid substitutions L234A and L235A. Preferably, the disabled Fc fragment comprises the amino acid substitutions L234A, L235A, and P329G.

Advantageously, the anti-C1-C6-VWF activity of the antibody or antigen-binding fragment thereof according to the first aspect of the invention means that it has significant utility as a therapeutic agent in its own right, and may be used in the treatment, amelioration or prevention of a condition caused by platelet-mediated aggregation, such as a thrombotic-related condition.

Accordingly, in a second aspect of the invention, there is provided an antibody or antigen-binding fragment thereof according to the first aspect, for use in therapy.

In a third aspect of the invention, there is provided an antibody or antigen-binding fragment thereof according to the first aspect, for use in treating, preventing or ameliorating a condition caused by platelet-mediated aggregation.

According to a fourth aspect of the invention, there is provided a method of treating, preventing or ameliorating a condition caused by platelet-mediated aggregation in a subject, the method comprising administering, or having administered, to a patient in need of such treatment, a therapeutically effective amount of an antibody or antigen-binding fragment thereof according to the first aspect.

The condition caused by platelet-mediated aggregation may be selected from the group consisting of: a thrombotic-related condition; thrombotic thrombocytopenia purpura (TTP) (also referred to as acquired thrombotic thrombocytopenia purpura (aTTP) or immune thrombotic thrombocytopeniarpura (iTTP) or congenital thrombotic thrombocytopenia purpura (cTTP)), acute coronary syndrome (ACS), atherosclerosis, ischemic stroke, atrial fibrillation (AF), acute myocardial infarction (AMI), cardiovascular disease (CVD), thrombosis, unstable angina, stable angina, angina pectoris, embolus formation, deep vein thrombosis, haemolytic uremic syndrome, haemolytic anaemia, acute renal failure, thrombolytic complications, disseminated intravascular coagulation, coronary heart disease, thromboembolic complications, restenosis, chronic unstable angina, peripheral vascular disease, arterial thrombosis, pre-eclampsia, embolism, restenosis, sepsis, vascular inflammation, glomerulonephritis, and thrombotic condition resulting from a coronavirus infection.

Preferably, the use or method in treating, preventing or ameliorating a condition caused by platelet-mediated aggregation comprises inhibiting platelet binding under conditions of high shear rate, i.e. pathological conditions.

The gradient in the blood flow speed (slope of the velocity profile) in the laminar layers is highest at the vessel wall. This shear rate is termed wall shear rate. Under normal physiological flow conditions, the wall shear rate increases from about 10 s⁻¹ in veins to about 15000 s⁻¹ in the smallest arteries, whereas maximal wall shear rates up to 40,000 s⁻¹ have been described for severe atherosclerotic arteries. One possible method for measuring shear rate in vivo, is further discussed in Brands et al., 1999, “An integrated system for the non-invasive assessment of vessel wall and hemodynamic properties of large arteries by means of ultrasound”. Specifically, a system referred to as arterial laboratory (ART-lab), measures radio frequency ultrasound signals to determine haemodynamic properties of arteries. The radio frequency signals received from an echo scanner are acquired by means of a data acquisition system and are then stored on a hard-disk. The assessment of blood flow velocity is based on the estimation of the temporal and spatial mean frequency in a given estimation window, which includes radiofrequency-samples filtered by a clutter filter, to discriminate between reflection and scattering. The temporal and spatial mean frequencies are directly related to velocity by means of the Doppler equation. From this velocity profile, the wall shear rate is calculated based on the maximum value of the derivative of the observed spatial velocity distribution with respect to the radius.

It will be appreciated that antibodies or antigen-binding fragments thereof according to the invention (referred to herein as “agents”) may be used in a monotherapy (e.g. the use of an antibody or antigen-binding fragment thereof alone), for treating, ameliorating or preventing a condition caused by platelet-mediated aggregation. Alternatively, agents according to the invention may be used as an adjunct to, or in combination with, known therapies for treating, ameliorating, or preventing a condition caused by platelet-mediated aggregation, such as aspirin, clopidogrel, abciximab, heparin, warfarin, and direct oral anticoagulants (DOACs) including, dabigatran (Pradaxa), rivaroxaban (Xarelto), apixaban (Eliquis), edoxaban (Savaysa), and betrixaban (Bevyxxa).

The agents according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising agents of the invention may be used in a number of ways. For instance, oral administration may be required, in which case the agents may be contained within a composition that may, for example, be ingested orally in the form of a tablet, capsule or liquid. Compositions comprising agents and medicaments of the invention may be administered by inhalation (e.g. intranasally). Compositions may also be formulated for topical use. For instance, creams or ointments may be applied to the skin. Alternatively, the agent may be delivered by gene therapy, for example through a AAV vector.

Agents and medicaments according to the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with agents used according to the invention is required and which would normally require frequent administration (e.g. at least daily injection).

In a preferred embodiment, agents and medicaments according to the invention may be administered to a subject by injection into the blood stream or directly into a site requiring treatment. Injections may be intravenous (bolus or infusion) or subcutaneous (bolus or infusion), or intradermal (bolus or infusion).

It will be appreciated that the amount of the antibody or antigen-binding fragment thereof (i.e. agent) that is required is determined by its biological activity and bioavailability, which in turn depends on the mode of administration, the physiochemical properties of the agent, and whether it is being used as a monotherapy or in a combined therapy. The frequency of administration will also be influenced by the half-life of the agent within the subject being treated. Optimal dosages to be administered may be determined by those skilled in the art, and will vary with the particular agent in use, the strength of the pharmaceutical composition, the mode of administration, and the advancement of the thrombotic-related condition. Additional factors depending on the particular subject being treated will result in a need to adjust dosages, including subject age, weight, gender, diet, and time of administration.

Generally, a daily dose of between 0.01 ug/kg of body weight and 100 mg/kg of body weight of agent according to the invention may be used for treating, ameliorating, or preventing a thrombotic-related condition, depending upon which agent. More preferably, the daily dose of agent is between 1 ug/kg of body weight and 100 mg/kg of body weight, more preferably between 10 ug/kg and 10 mg/kg body weight, and most preferably between approximately 100 ug/kg and 10 mg/kg body weight.

The agent may be administered before, during or after onset of a thrombotic-related condition. Daily doses may be given as a single administration (e.g. a single daily injection). Alternatively, the agent may require administration twice or more times during a day. As an example, agents may be administered as two (or more depending upon the severity of the thrombotic-related condition being treated) daily doses of between 0.07 μg and 700 mg (i.e. assuming a body weight of 70 kg). A patient receiving treatment may take a first dose upon waking and then a second dose in the evening (if on a two dose regime) or at 3- or 4-hourly intervals thereafter. Alternatively, the agent may be administered less frequently, such as once every two days, once every week, or once every two weeks. Alternatively, a slow release device may be used to provide optimal doses of agents according to the invention to a patient without the need to administer repeated doses. Known procedures, such as those conventionally employed by the pharmaceutical industry (e.g. in vivo experimentation, clinical trials, etc.), may be used to form specific formulations of the agents according to the invention and precise therapeutic regimes (such as daily doses of the agents and the frequency of administration).

In a fifth aspect of the invention, there is provided a pharmaceutical composition comprising an antibody or antigen-binding fragment thereof according to the first aspect, and optionally a pharmaceutically acceptable vehicle.

The pharmaceutical composition is preferably anti-thrombotic, i.e. a pharmaceutical formulation used in the therapeutic amelioration, prevention or treatment of a condition caused by platelet-mediated aggregation.

The invention also provides in a sixth aspect, a process for making the pharmaceutical composition according to the fifth aspect, the process comprising combining a therapeutically effective amount of an antibody or antigen-binding fragment thereof as defined in the first aspect, with a pharmaceutically acceptable vehicle.

The antibody or antigen-binding fragment thereof may be as defined with respect to the first aspect. Preferably, the antibody or antigen-binding fragment thereof is one of the antibodies from FIG. 6, or an antigen-binding fragment thereof.

A “subject” may be a vertebrate, mammal, or domestic animal. Hence, medicaments according to the invention may be used to treat any mammal, for example livestock (e.g. a horse), pets, or may be used in other veterinary applications. Most preferably, the subject is a human being.

A “therapeutically effective amount” of the antibody or antigen-binding fragment thereof is any amount which, when administered to a subject, is the amount of agent that is needed to treat the thrombotic-related condition, or produce the desired effect.

For example, the therapeutically effective amount of antibody or antigen-binding fragment thereof used may be from about 0.1 ng/kg to about 100 mg/kg, and preferably from about 1 ng/kg to about 10 mg/kg. It is preferred that the amount of antibody or antigen-binding fragment thereof is an amount from about 10 ng/kg to about 10 mg/kg, and most preferably from about 50 ng/kg to about 5 mg/kg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The active agent according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The agent may be prepared as a sterile solid composition that may be dissolved or suspended at the time of administration using sterile water, saline, or other appropriate sterile injectable medium.

The agents and compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The agents used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

In a seventh aspect of the invention, there is provided a polynucleotide sequence encoding the antibody, or antigen-binding fragment thereof as defined in the first aspect.

In an eighth aspect of the invention, there is provided an expression cassette comprising a polynucleotide sequence according to the seventh aspect.

The polynucleotide sequence encoding the antibody, or antigen-binding fragment of the invention is preferably harboured in a recombinant vector, for example a recombinant vector for delivery into a host cell of interest to enable production of the antibody, or antigen-binding fragment thereof.

Accordingly, in a ninth aspect of the invention, there is provided a recombinant vector comprising the expression cassette according to the eighth aspect.

The vector encoding the antibody, or antigen-binding fragment may for example be a plasmid, cosmid or phage and/or be a viral vector. Such recombinant vectors are highly useful in the delivery systems of the invention for transforming cells with the nucleotide sequences. The nucleotide sequences may preferably be a DNA sequence, and it is this DNA sequence which encodes the antibody, or antigen-binding fragment.

Recombinant vectors encoding the antibody, or antigen-binding fragment may also include other functional elements. For example, they may further comprise a variety of other functional elements including a suitable promoter for initiating transgene expression upon introduction of the vector in a host cell. For instance, the vector is preferably capable of autonomously replicating in the nucleus of the host cell. In this case, elements which induce or regulate DNA replication may be required in the recombinant vector. Alternatively, the recombinant vector may be designed such that it integrates into the genome of a host cell. In this case, DNA sequences which favour targeted integration (e.g. by homologous recombination) are envisaged. Suitable promoters may include the SV40 promoter, CMV, EF1a, PGK, viral long terminal repeats, as well as inducible promoters, such as the Tetracycline inducible system, as examples. The cassette or vector may also comprise a terminator, such as the Beta globin, SV40 polyadenylation sequences or synthetic polyadenylation sequences. The recombinant vector may also comprise a promoter or regulator or enhancer to control expression of the nucleic acid as required.

The vector may also comprise DNA coding for a gene that may be used as a selectable marker in the cloning process, i.e. to enable selection of cells that have been transfected or transformed, and to enable the selection of cells harbouring vectors incorporating heterologous DNA. For example, ampicillin, neomycin, puromycin or chloramphenicol resistance is envisaged. Alternatively, the selectable marker gene may be in a different vector to be used simultaneously with the vector containing the transgene. The cassette or vector may also comprise DNA involved with regulating expression of the nucleotide sequence, or for targeting the expressed polypeptide to a certain part of the host cell.

Purified vector may be inserted directly into a host cell by suitable means, e.g. direct endocytotic uptake. The vector may be introduced directly into a host cell (e.g. a eukaryotic or prokaryotic cell) by transfection, infection, electroporation, microinjection, cell fusion, protoplast fusion, calcium phosphate, cationic lipid-based lipofection, polymer or dendrimer-based methods or ballistic bombardment.

Alternatively, vectors of the invention may be introduced directly into a host cell using a particle gun.

Alternatively, the delivery system may provide the polynucleotide to the host cell without it being incorporated in a vector. For instance, the nucleic acid molecule may be incorporated within a liposome or virus particle. Alternatively a “naked” polynucleotide may be inserted into a host cell by a suitable means e.g. direct endocytotic uptake.

In a tenth aspect of the invention, there is provided a host cell comprising the polynucleotide sequence according to the seventh aspect, the expression cassette according to the eighth aspect, or the vector according to the ninth aspect.

The host cell may be a eukaryotic or prokaryotic host cell. Preferably, the host cell is a eukaryotic host cell. More preferably, the host cell is a mammalian host cell such as NSo murine myeloma cells, PER.C6® human cells, Human embryonic kidney 293 cells or Chinese hamster ovary (CHO) cells. Most preferably, the host cell is a CHO cell.

In an eleventh aspect, there is provided a method of preparing the antibody, or antigen-binding fragment thereof according to the first aspect, the method comprising:
a) introducing, into a host cell, the vector of the ninth aspect; and
b) culturing the host cell under conditions to result in the production of the antibody, or antigen-binding fragment thereof according to the first aspect.

The host cell of step a) may be a eukaryotic or prokaryotic host cell. Preferably, the host cell is a eukaryotic host cell. More preferably, the host cell is a mammalian host cell such as NSo murine myeloma cells, PER.C6® human cells, Human embryonic kidney 293 cells or Chinese hamster ovary (CHO) cells. Most preferably, the host cell is a CHO cell.

The method may further comprise (c) harvesting, centrifuging and/or filtering the cell culture media to obtain a cell culture supernatant comprising the antibody or antigen binding fragment thereof.

The method may further comprise (d) separating and purifying the antibody or antigen-binding fragment thereof from the cell culture supernatant. Preferably, purification is performed by at least one chromatographic step.

Suitable chromatographic steps include affinity chromatography and/or ion exchange chromatography. Preferably, affinity chromatography is protein A chromatography. Ion exchange chromatography may be anionic exchange chromatography and/or cationic exchange chromatography.

Preferably, step (d) comprises separating and purifying the antibody or antigen-binding fragment thereof from the cell culture supernatant by:
i) protein A chromatography;
ii) anionic exchange chromatography; and/or
iii) cationic exchange chromatography.

The method may further comprise (e) filtering the purified antibody or antigen-binding fragment thereof resulting from step (d). Preferably, step (e) comprises virus filtration. Thus, preferably the purified antibody or antigen-binding fragment thereof resulting from step (d) is filtered using a virus filtration membrane. Suitable membranes would be known to those skilled in the art.

Alternatively, in another aspect, there is provided an antibody or antigen-binding fragment thereof obtained by a method comprising selecting an antibody or antigen-binding fragment thereof that specifically binds to one or more of the C1, C2, C3, C4, C5, and/or C6 domain of VWF, using directed evolution or a computational approach.

As discussed herein, VWF expression is increased in a number of conditions caused by platelet-mediated aggregation, including ischemic stroke, heart attack, acquired thrombotic thrombocytopenia purpura and atrial fibrillation. Thus, given that the antibodies of the invention are able to bind to one or more of the C1, C2, C3, C4, C5, and/or C6 domain of VWF, the antibodies or antigen-binding fragments thereof may be used as a robust diagnostic tool by detecting the presence, and determining the concentration of, VWF.

Thus, in a twelfth aspect, there is provided the antibody or antigen-binding fragment thereof according to the first aspect, for use in diagnosis or prognosis.

According to a thirteenth aspect of the invention, there is provided the antibody or antigen-binding fragment thereof according to the first aspect, for use in diagnosing or prognosing a condition caused by platelet-mediated aggregation.

According to the fourteenth aspect, there is provided a method of diagnosing or prognosing a condition caused by platelet-mediated aggregation in a subject, the method comprising detecting VWF in a biological sample obtained from the subject with the antibody or antigen-binding fragment thereof according to the first aspect.

The condition caused by platelet-mediated aggregation may be selected from the group consisting of: a thrombotic-related condition; thrombotic thrombocytopenia purpura (TTP) (also referred to as acquired thrombotic thrombocytopeniaurpura (aTTP) or immune thrombotic thrombocytopeniarpura (iTTP) or congenital thrombotic thrombocytopenia purpura (cTTP)), acute coronary syndrome (ACS), atherosclerosis, ischemic stroke, atrial fibrillation (AF), acute myocardial infarction (AMI), cardiovascular disease (CVD), thrombosis, unstable angina, stable angina, angina pectoris, embolus formation, deep vein thrombosis, haemolytic uremic syndrome, haemolytic anaemia, acute renal failure, thrombolytic complications, disseminated intravascular coagulation, coronary heart disease, thromboembolic complications, restenosis, chronic unstable angina, peripheral vascular disease, arterial thrombosis, pre-eclampsia, embolism, restenosis, sepsis, vascular inflammation, glomerulonephritis, and thrombotic condition resulting from a coronavirus infection (e.g. COVID-19).

The method may be an in vitro or ex vivo method. Preferably, the method is an in vitro method.

The use or method may comprise determining the level of expression of VWF in a subject, preferably wherein an increase in the concentration of VWF in the biological sample when compared to a reference concentration from a healthy control population is indicative of a condition caused by platelet-mediated aggregation or a poor prognosis.

In one embodiment, a 1 fold increase of VWF when compared to the reference from a healthy control population is indicative of a condition caused by platelet-mediated aggregation or a poor prognosis. In one embodiment, a 2 fold, 3 fold, 4 fold or 5 fold increase of VWF when compared to the reference from a healthy control population is indicative of a condition caused by platelet-mediated aggregation or a poor prognosis. In one embodiment, a 10 fold, 50 fold or 100 fold increase of VWF when compared to the reference from a healthy control population is indicative of a condition caused by platelet-mediated aggregation or a poor prognosis.

According to the fifteenth aspect of the invention, there is provided a kit for diagnosing a subject suffering from a condition caused by platelet-mediated aggregation, or for providing a prognosis of the subject's condition, the kit comprising an antibody or antigen-binding fragment thereof according to the first aspect for detecting VWF in a sample from a test subject.

The kit may further comprise instructions for use and/or a receptacle for obtaining a biological sample from a subject.

Preferably, the condition caused by platelet-mediated aggregation is as defined above.

Prognosis may relate to determining the therapeutic outcome in a subject that has been diagnosed with a condition caused by platelet-mediated aggregation. Prognosis may relate to predicting the rate of progression or improvement and/or the duration of a condition caused by platelet-mediated aggregation in a subject, the probability of survival, and/or the efficacy of various treatment regimes. Thus, a poor prognosis may be indicative of progression of a condition caused by platelet-mediated aggregation, low probability of survival and reduced efficacy of a treatment regime. A favourable prognosis may be indicative of resolution of a condition caused by platelet-mediated aggregation, high probability of survival and increased efficacy of a treatment regime.

Preferably, the sample comprises a biological sample. The sample may be any material that is obtainable from a subject from which protein is obtainable.

The biological sample may be tissue or a biological fluid. The biological sample may be any material that is obtainable from the subject from which blood plasma, endothelial cells, megakaryocytes, and platelets are obtainable. Furthermore, the sample may be blood, plasma, serum, spinal fluid, urine, sweat, saliva, tears, breast aspirate, breast milk, prostate fluid, seminal fluid, vaginal fluid, stool, cervical scraping, cytes, amniotic fluid, intraocular fluid, mucous, moisture in breath, animal tissue, cell lysates, tumour tissue, hair, skin, buccal scrapings, lymph, interstitial fluid, nails, bone marrow, cartilage, prions, bone powder, ear wax, lymph, granuloma, cancer biopsy or combinations thereof.

The sample may be a liquid aspirate. For example, the sample may be bronchial alveolar lavage (BAL), ascites, pleural lavage, or pericardial lavage.

The sample may comprise blood, urine, tissue etc. In one preferred embodiment, the biological sample comprises a blood sample. The blood may be venous or arterial blood. Blood samples may be assayed immediately. Alternatively, the blood sample may be stored at low temperatures, for example in a fridge or even frozen before the method is conducted. Alternatively, the blood sample may be stored at room temperature, for example between 18 to 22 degrees Celsius, before the method is conducted. The blood sample may comprise comprises blood serum. The blood sample may comprise blood plasma. Preferably, however the detection is carried out on whole blood and most preferably the blood sample is peripheral blood.

The blood may be further processed before the use of the first aspect is performed. For instance, an anticoagulant, such as citrate (such as sodium citrate), hirudin, heparin, PPACK, or sodium fluoride may be added. Thus, the sample collection container may contain an anticoagulant in order to prevent the blood sample from clotting.

Preferably, the sample may comprise blood plasma, endothelial cells, megakaryocytes, and/or platelets.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1-114 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and Tis the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:−Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in, for example, in those of SEQ ID Nos: 1 to 114 that are amino acid sequences.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

FIG. 1 provides a schematic of VWF peptide structure showing the propeptide and mature VWF regions. Platelet binding is predominantly via the A1 and A3 domains, which are targeted by current therapies, including Caplacizumab. The antibodies or antigen-binding fragments thereof of the invention target one or more of the C1-C6 domains.

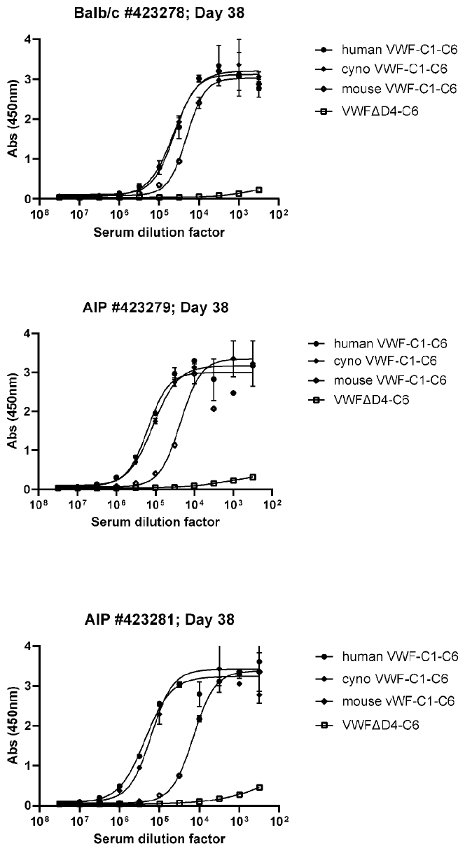

FIG. 2 shows mouse serum reactivity to VWF proteins. 38 days after the primary immunisation, blood was withdrawn and reactivity to VWF proteins was determined by ELISA. Serum from immunised mice showed good reactivity towards human, mouse and cynomolgus recombinantly produced VWF-C1-C6 protein. There was minimal background reactivity to a recombinant fragment of VWF with C1-C6 domains deleted (VWFΔD4-C6). Mice #423277 and #423281 were selected for harvest and storage of splenocytes and bone marrow.

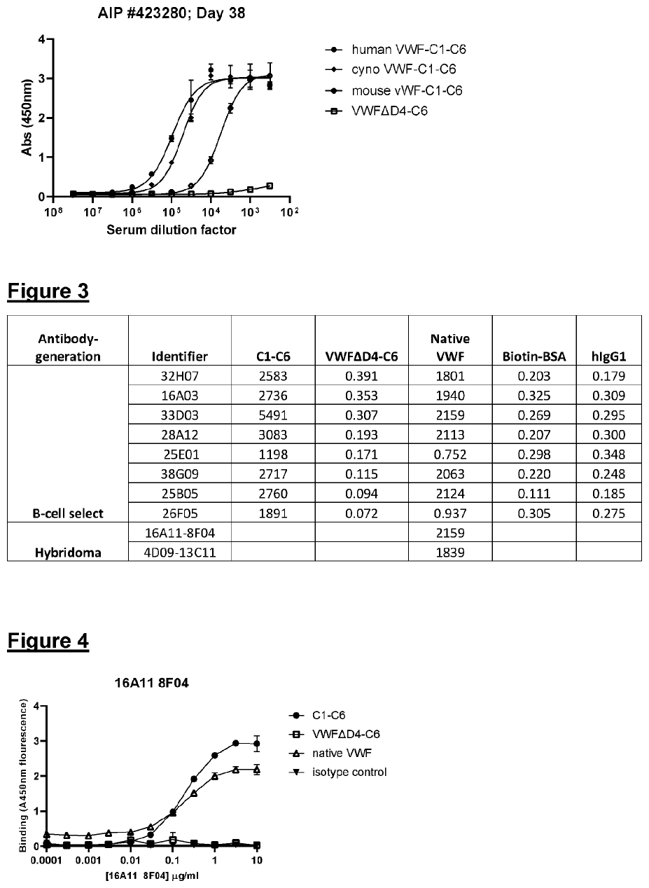

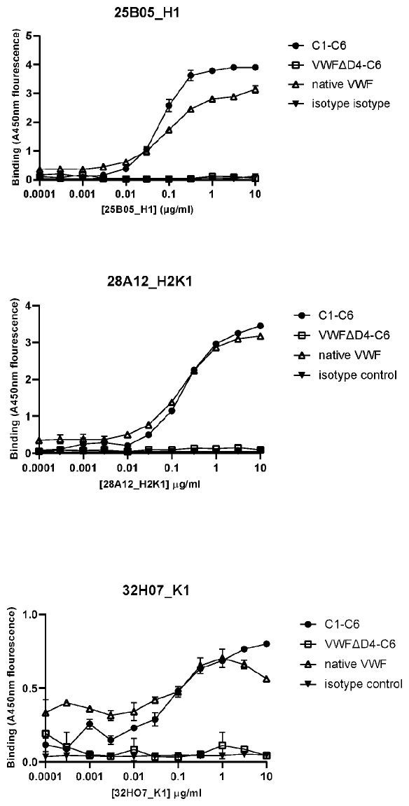

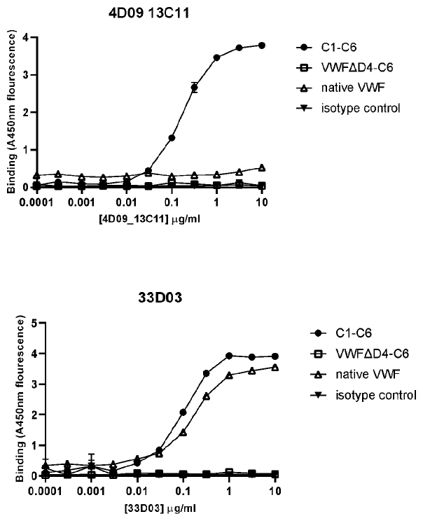

FIG. 3 shows ELISA-based reactivity screening of primary hybridomas and B-cell selections. Hybridoma and B-cell selections were conducted on the two mice selected from the plasma reactivity screening. Ten potential clones showing cross-reactivity between human, macaca (cynomolgus monkey) and mouse VWF C1-C6 recombinant protein were identified by ELISA for sub-cloning and generation of mAbs.

FIG. 4 shows ELISA binding of unique mAbs to native VWF protein and recombinant C1-C6 protein. The six unique sequences were cloned into an IgG1 human expression vector and expressed as monoclonal antibodies. Binding to recombinant human C1-C6 and/or native human VWF was confirmed by ELISA. All mAbs showed minimal binding reactivity to the isotype control and VWF without the C1-C6 domain (VWFΔD4-C6). Clone 28A12_H2K1 demonstrated equivalent binding to both C1-C6 and native VWF. 4D09_13C11 showed preferential binding to C1-C6 protein, with little binding to native VWF. All remaining clones showed similar binding to C1-C6 and native VWF.

FIG. 5 shows ELISA binding of unique mAbs to cynomolgus monkey recombinant VWF C1-C6 protein. The six unique sequences were cloned into an IgG1 human expression vector and expressed as monoclonal antibodies. Binding to human and cynomolgus VWF-C1-C6 was confirmed by ELISA. All clones were reactive to both human and cynomolgus VWF-C1-C6 by ELISA. All clones showed minimal binding to VWF without the C1-C6 domain (VWFΔD4-C6).

FIG. 6 is a table showing the various sequences of six embodiments of the anti-VWF antibody of the invention. CDR=complementarity determining region; VH=variable heavy chain sequence; VL=variable light chain sequence; HC=constant heavy chain sequence; LC=constant light chain sequence.

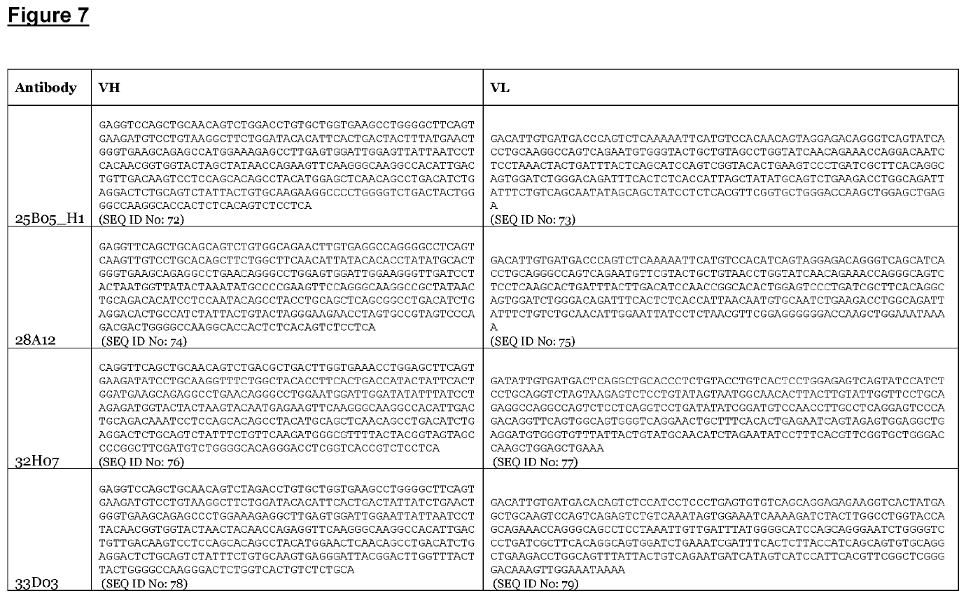

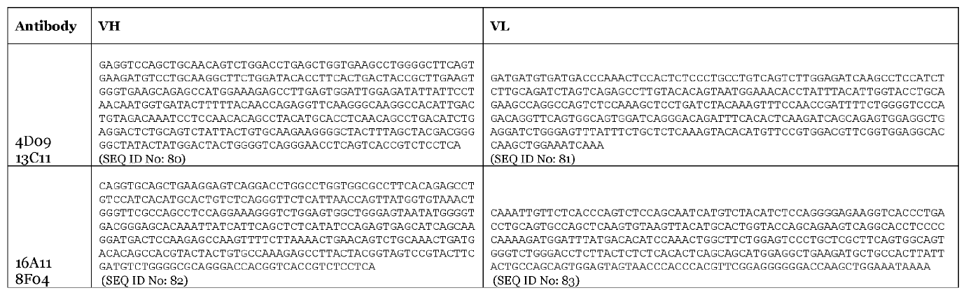

FIG. 7 is a table showing the nucleotide sequences encoding the VH and VL regions of six embodiments of the anti-VWF antibodies according to the invention.

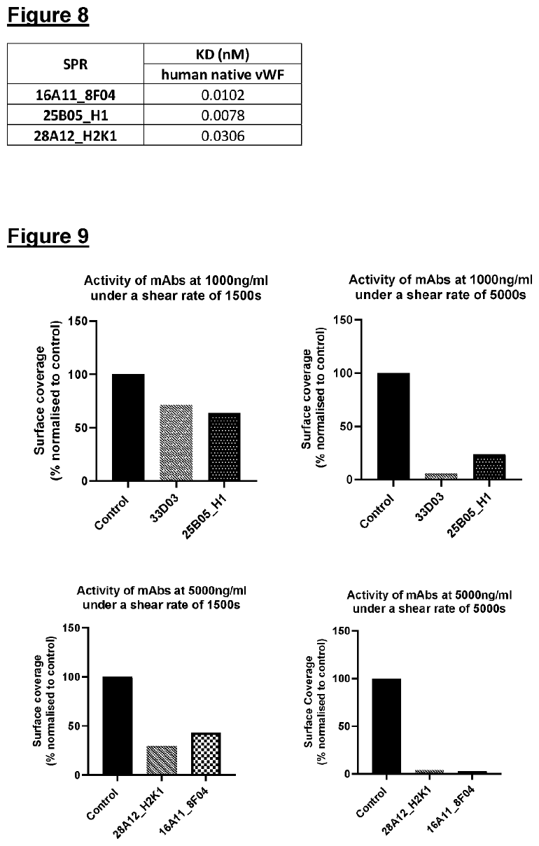

FIG. 8 is a table showing KD determination to human native VWF for the selected monoclonal antibodies. Three clones were selected for affinity determination (KD) for binding to native human VWF. All tested clones showed sub nM affinities for binding to native human VWF protein.

FIG. 9 shows the results of selected anti-VWF C1-C6 antibodies in a whole blood flow assay under normal (1500s) and high shear (5000s) rates. 33D03, 25B05_H1, 28A12_H2K1 and 16A11_8F04 antibodies inhibit platelet capture under high shear rate (5000s⁻¹) to a greater extent than under normal shear rate conditions (1500s⁻¹) in a whole blood flow assay, indicating that these antibodies can block the prothrombotic function of VWF while maintaining its normal haemostatic function.

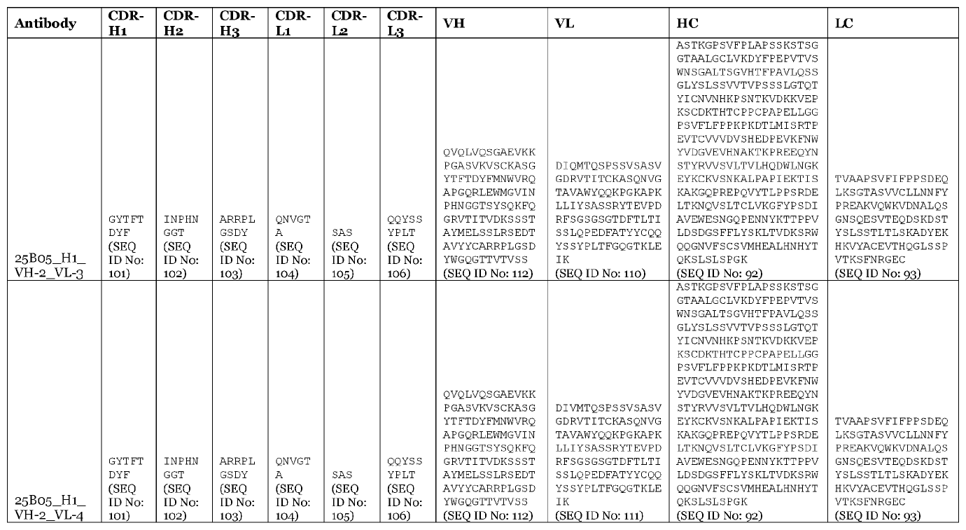

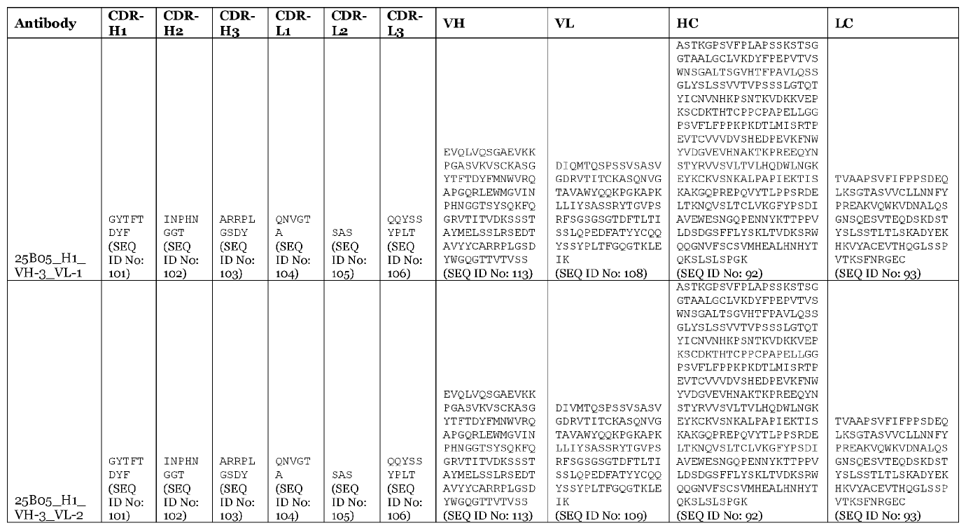

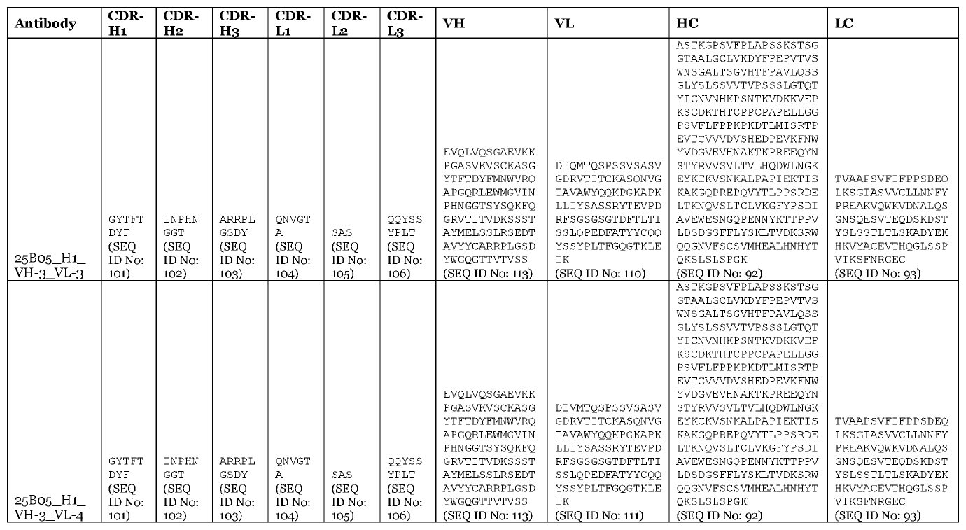

FIG. 10 shows the results of Octet epitope binning of the selected monoclonal antibodies. Epitope binning is a technique used to cluster different monoclonal antibodies by the specific region on the antigen that is recognised by the antibody, the epitope. The table shows data interpretation for the Octet epitope binning experiment, where none of the antibodies tested displayed competition for binding, indicating that the antibodies may have different binding epitopes. NC=no competition; NA=not applicable.

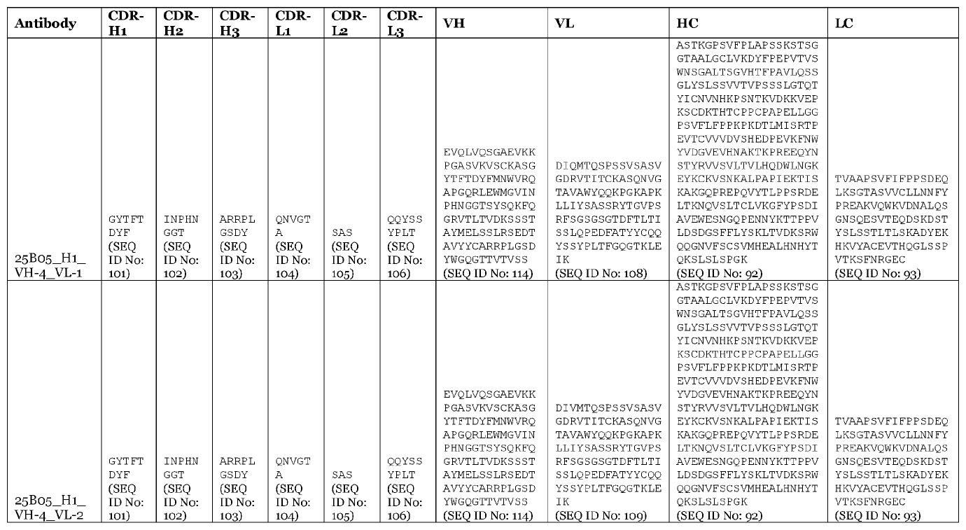

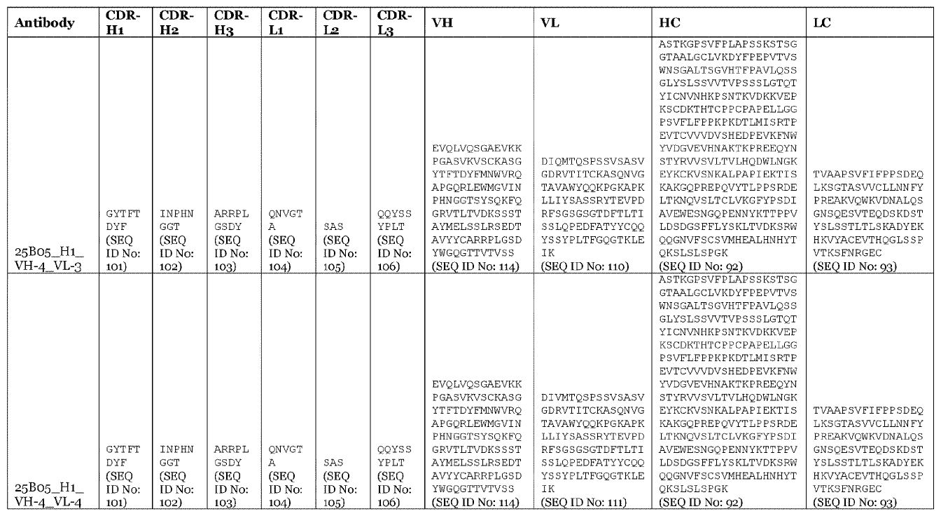

FIG. 11 is a table showing the various sequences of the humanised anti-VWF antibodies according to the invention.

### EXAMPLES

The accumulation of VWF has been associated with an increased risk to a number of conditions caused by platelet-mediated aggregation, including various cardiovascular diseases and thrombotic-related conditions. Currently, however, therapies target the essential A1 domain for VWF, inhibiting platelet binding required for haemostasis, leading to an increased bleeding risk in patients. Therefore, the inventors set out to inhibit a previously untargeted region of VWF, i.e. the C1-C6 domains. The inventors developed antibodies that are capable of specifically binding to the C1-C6 region (i.e. one or more of the C1-C6 domains as shown in FIG. 1), providing an improved treatment for a number of thrombotic-related conditions. As discussed below, several novel C1-C6 targeting antibodies have been produced, which could therefore be used in therapy and diagnosis. Additionally, the inventors have also produced several humanised antibodies which exhibit the desired effects (i.e. strong binding affinity for human VWF), and which could therefore be used in therapy and diagnosis.

### Example 1—Generation of Anti-Human VWF mAbs

Anti-human VWF mAbs were generated from mice (BALB/c or AIP) immunised with human VWF C1-C6-GST (Table 1). Mice were immunised with antigen and boosted between one and three times at approximately one-month intervals (Table 1).

TABLE 1
Bi-weekly immunisation strategy

| | Mouse (n = 5) |
|---|---|
| Blood withdrawal | Day 0 |
| Primary immunization (s.c.) | C1-C6-GST |
| day 1 | |
| Boost 1 (s.c.) Day 14 | C1-C6-Fc |
| Boost 2 (s.c.) Day 28 | C1-C6-GST |
| blood withdrawal and serum | Day 38 |
| titration | |

Balb/c or AIP mice were immunised with recombinantly produced human VWF C1-C6-GST protein. After 14 days the mice received the first boost human VWF C1-C6-Fc and after 28 days, the animals received their second boost of human VWF C1-C6-GST. Blood was withdrawn for analysis 38 days after the first immunisation.

### Example 2—Mouse Serum Reactivity to VWF Proteins

ELISA-based serum reactivity screening of immunised mice was conducted towards human, mouse and cynomolgus recombinantly produced VWF-C1-C6 protein. ELISA plates were coated overnight with all targets coated at 1 ug/ml, blocked with 1% BSA in PBS and subsequently incubated with a semi-log dilution series of mouse immune sera (starting dilution 1:316 or 1:1000, 11 dilutions, in duplicate). Detection was carried out using anti-mouse IgG-HRP and visualized using TMB. Average A450 signal ±sd of measured samples is shown.

As illustrated in FIG. 2, the serum from immunised mice showed good reactivity towards human, mouse and cynomolgus recombinantly produced VWF-C1-C6 protein. There was minimal background reactivity to a recombinant fragment of VWF with C1-C6 domains deleted (VWFΔD4-C6).

### Example 3—ELISA-Based Reactivity Screening of Primary Hybridomas and B-Cell Selections

Hybridomas were either generated and cloned using the ClonaCell-HY hybridoma cloning kit (StemCell Technologies, Vancouver, BC) or using conventional methods. In the conventional method, B cells from the spleens of the immunised animals were fused, by electrofusion, with NS-1 myeloma cells. After overnight recovery, fused cells were plated at limiting dilution in 96-well plates and subjected to hypoxanthine-aminopterin thymidine selection. Hybridoma culture supernatants were examined for the presence of anti-VWF antibodies by ELISA (FIG. 3). Immune serum activity was observed from two hybridomas, which were selected for subcloning and generation of chimeric antibodies.

B-cell selections were conducted by panning of splenocytes towards an irrelevant IgG protein, an off-target protein and a positive selection on VWF protein. Selected B-cells were seeded into 96-well plates, cultured for 8 days, and supernatant reactivity to VWF C1-C6 and native VWF was screened using an ELISA. Immune serum activity was observed from eight B-cell selections, which were selected for subcloning and generation of chimeric antibodies.

### Example 4—ELISA Binding of Unique mAbs to Native VWF and Recombinant C1-C6 Protein

RNA was isolated from TRIzol samples. cDNA was generated and amplification of V-regions was completed according to standard procedures. PCR amplification of VH- and VL-gene fragments using standard procedures or by using degenerate forward primers. Amplified V-genes were gel purified and cloned into human IgG1/IgK vectors using T4 ligase for DNA sequencing. Ligation mixes (~25 ng vector) were transformed into chemocompetent E. coli XL1-Blue cells. Miniprep DNA was isolated from full-length insert containing clones. Isolated DNA (up to 10 VH and VL fragments per hybridoma) was sequenced and analysed using standard methods. Plasmids containing the correct genes were stored as glycerol stocks.

The DNA expression constructs encoding the chimeric antibody were prepared using restriction sites for cloning into mammalian expression vectors as well as a human signal sequence. BsiWI and BsmI restriction sites were introduced to frame the variable domains containing the signal sequence for cloning into mammalian expression vectors containing the human γ 1 or human kappa constant regions.

The correct clones were confirmed using DNA sequencing. Plasmid DNA was transfected into HEK293 cells using FectoPro. Supernatants were harvested after ~5 days. Antibody concentration was measured in culture supernatant the yield was calculated using Octet or ELISA. From the original ten selected clones (two from hybridomas and eight from B-cell selections), two clones were removed due to sequencing errors and two were removed due to low reactivity in the ELISA. The six remaining clones were progressed to full mAbs.

Antibodies were purified via protein A (Mab Select SuRe) resin and antibody concentrations were measured using Nanodrop. Antibody integrity and purity was confirmed using reducing SDS-PAGE and SEC-HPLC. Antibody target reactivity was determined using ELISA.

ELISA-based reactivity screening of generated mAbs was conducted towards human C1-C6, VWFΔD4-C6, native VWF and an isotype control. ELISA plates were coated overnight with all targets coated at 1 ug/ml, blocked with 1% BSA in PBS and subsequently incubated with a semi-log dilution series of purified antibodies (starting concentration 10 ug/ml, in duplicate). Detection was carried out using anti-human-Ig-kappa HRP and visualized using TMB. Average A450 signal ±SD of measured samples is shown.

As illustrated in FIG. 4, the six mAbs demonstrated binding to recombinant C1-C6 and/or native human VWF. Additionally, all mAbs showed minimal binding reactivity to the isotype control and VWF without the C1-C6 domain (VWFΔD4-C6). Clone 28A12_H2K1 demonstrated equivalent binding to both C1-C6 and native VWF. 4D09_13C11 showed preferential binding to C1-C6 protein, with little binding to native VWF. All remaining clones showed similar binding to C1-C6 and native VWF.

### Example 5—ELISA Binding of Unique mAbs to Cynomolgus Monkey Recombinant VWF C1-C6 Protein

ELISA-based reactivity screening of generated mAbs was conducted towards human VWF C1-C6, VWFΔD4-C6, and cynomolgus VWF-C1-C6 proteins. ELISA plates were coated overnight with all targets coated at 1 μg/ml, blocked with 1% BSA in PBS and subsequently incubated with a semi-log dilution series of purified antibodies (starting concentration 10 μg/ml, in duplicate). Detection was carried out using anti-human-Ig-kappa HRP and visualized using TMB. Average A450 signal ±SD of measured samples is shown.

As illustrated in FIG. 5, all clones were reactive to both human and cynomolgous VWF-C1-C6 by ELSA. Additionally, all clones showed minimal binding to VWF without the C1-C6 domain (VWFΔD4-C6).

### Example 6—Binding of Anti-VWF Antibodies to Human Native VWF as Determined by SPR

Antibodies were captured on Protein A sensor chip. The system was purged using running buffer (10 mM HEPES pH 7.4, 300 mM NaCl, 3 mM EDTA, 0.05% P20) and a series S Protein A chip was docked in the Biacore T200. The surface was conditioned with 10 mM glycine-HCl pH 1.5 regeneration solution (3 injections). Each antibody was diluted to ~0.8 μg/ml in running buffer to capture ~350 RU on flow cells 2, 3 and 4 (flow cell 1 used as in-line reference cell). Single-cycle kinetic analysis of the native human VWF protein binding to the antibodies was performed using the following parameters:
Flow cell 1-4
Flow rate (μl/min) 30
Sample compartment temperature 10 (° C.)
Flow cell temperature (° C.) 25
Contact time(s) 120
Dissociation time(s) 7200
Protein concentrations (nM)
   500, 166.67, 55.56, 18.52, 6.17
   100, 33.33, 11.11, 3.70, 1.23
   20, 6.67, 2.22, 0.74, 0.25

The chip surface was regenerated after each cycle with 10 mM glycine-HCl pH 1.5 for 30 s at 50 μl/min. Affinities are reported for each antibody tested against human native VWF. As illustrated in FIG. 8, all of the tested antibody clones showed sub nM affinities for binding to native human VWF protein.

### Example 7—Whole Blood Flow Assay

Slides were coated with collagen, perfused with whole blood with test antibody at concentrations as indicated. Surface coverage was calculated from the mean fluorescent intensity from platelet images taken after five minutes of blood flow and normalised to the isotype control, presented as percentage.

As illustrated in FIG. 9, 33D03, 25B05_H1, 28A12_H2K1 and 16A11_8F04 antibodies inhibit platelet capture under high shear rate (5000s⁻¹) to a greater extent than under normal shear rate conditions (1500s⁻¹) in a whole blood flow assay, indicating that these antibodies can block the pro-thrombotic function of VWF while maintaining its normal haemostatic function.

### Example 8—Octet Epitope Binding

Pre-wetted biosensors were loaded with VWF C1-C6-Fc (20 μg/ml) or no protein followed by an association of Analyte #1 (>15 μg/ml) or Analyte 2 (>15 μg/ml). Each antibody was tested in both orientations as either Analyte #1 (bound to VWF C1-C6-Fc protein on the biosensor) or Analyte 2 (added to the analyte 1 bound to biosensor) using the Octet system. Measurement of association and dissociation by the Octet allowed an assessment of competition of binding. All assay steps were performed at 1000 rpm, 30° C.

As illustrated in FIG. 10, none of the antibodies tested displayed competition for binding, indicating that the antibodies may have different binding epitopes.

### Example 9—Humanised Variants

The inventors next set out to generate humanised variants and to determine the EC50 of the humanised antibodies for human VWF.

### Method: Cloning and Expression of the Humanised Variants

Humanised antibody variant sequences were generated by in silico design. DNA expression constructs encoding the humanised antibody variants were prepared de novo by build-up of overlapping oligonucleotides including restriction sites for cloning into mammalian expression vectors as well as a human signal sequence. Two restriction sites were introduced to frame the VH domain containing the signal sequence for cloning into mammalian expression vectors containing the human γ constant region. Restriction sites were introduced to frame the VL domain containing the signal sequence for cloning into mammalian expression vector containing the human kappa constant region. Expression plasmids encoding the heavy and light chains respectively were transiently co-transfected into HEK 293 6E cells and expressed to produce antibody protein. Preparations were purified using protein A and concentrations were measured using a Nanodrop (Thermo Scientific).

### Method: EC50 Determination (by ELISA)

ELISA-base reactivity screening of purified antibodies. ELISA plates were coated overnight with human VWF protein. The plates were blocked with 1% BSA in PBS and subsequently incubated with a semi-log dilution series of purified antibodies. Detection was carried out using anti-human-Ig, κ-HRP and visualized using TMB. EC50 values were calculated from average A450 signals using a four-parameter logistical regression curve fitting model.

TABLE 2
EC50 binding data of humanised variants

| Humanised mAb | EC50 (ng/ml) human VWF |
|---|---|
| 33D03_par | 49.8 |
| 33D03_H1-L1 | 43.7 |
| 33D03_H1-L2 | 45.3 |
| 33D03_H1-L3 | 59.7 |
| 33D03_H1-L4 | 54.0 |
| 33D03_H2-L1 | 37.3 |
| 33D03_H2-L2 | 39.4 |
| 33D03_H2-L3 | 37.1 |
| 33D03_H2-L4 | 38.1 |
| 33D03_H3-L1 | 44.7 |
| 33D03_H3-L2 | 45.7 |
| 33D03_H3-L3 | 48.3 |
| 33D03_H3-L4 | 43.4 |
| 33D03_H4-L1 | 38.1 |
| 33D03_H4-L2 | 35.4 |
| 33D03_H4-L3 | 37.1 |
| 33D03_H4-L4 | 36.5 |
| 33D03_H5-L1 | 29.7 |
| 33D03_H5-L2 | 29.4 |
| 33D03_H5-L3 | 33.5 |
| 33D03_H5-L4 | 36.5 |
| 25B05_H1-L1 | 31.2 |
| 25B05_H1-L2 | 33.4 |
| 25B05_H1-L3 | 33.5 |
| 25B05_H1-L4 | 34.4 |
| 25B05_H2-L1 | 61.2 |
| 25B05_H2-L2 | 56.4 |
| 25B05_H2-L3 | 41.6 |
| 25B05_H2-L4 | 31.2 |
| 25B05_H3-L1 | 59.4 |
| 25B05_H3-L2 | 51.6 |
| 25B05_H3-L3 | 43.4 |
| 25B05_H3-L4 | 37.9 |
| 25B05_H4-L1 | 38.5 |
| 25B05_H4-L2 | 35.7 |
| 25B05_H4-L3 | 27.1 |
| 25B05_H4-L4 | 26.4 |

As illustrated in Table 2 above, the inventors have demonstrated that the humanised antibodies according to the invention have strong binding affinity for human VWF.

### DISCUSSION & CONCLUSIONS

The inventors have identified the C1-C6 domains of the VWF protein as being important for VWF function in pro-thrombotic, pathological conditions, and have therefore developed antibodies that are capable of binding to, and inhibiting, C1-C6 VWF function. For example, as shown in FIGS. 4 and 5, the inventors have developed a number of antibodies and antigen-binding fragments thereof that have demonstrated the ability to specifically target the C1-C6 domains of VWF. Additionally, the inventors have generated humanised versions of the antibodies according to the invention, and have demonstrated that the humanised antibodies can bind to human VWF with high affinity.

The current anti-VWF therapies target and inhibit the A1 domain of VWF, and as such, block an essential haemostatic function of VWF. This blocks clotting under normal conditions as well as pathological conditions, resulting in a severe bleeding risk in patients. The inventors have identified novel antibodies that can target the C1-C6 domains of VWF, which are primarily involved in maintaining the role of VWF under pathological conditions. For example, as illustrated in FIG. 9, the antibodies inhibit platelet capture under high shear rate (5000s⁻¹) to a greater extent than under normal shear rate conditions (1500s⁻¹), indicating that the antibodies can block the pro-thrombotic function of VWF while maintaining its normal haemostatic function. Accordingly, the inventors have identified a novel strategy for preventing or treating thrombotic-related conditions, without blocking the essential haemostatic function of VWF under normal conditions.

1. An antibody or antigen-binding fragment thereof that specifically binds to one or more of a C1, C2, C3, C4, C5, and/or C6 domain of Von Willebrand Factor (VWF).

2. The antibody or antigen-binding fragment thereof according to claim 1, wherein the antibody or antigen-binding fragment thereof comprises:
(i) a CDR-H1 domain comprising or consisting of SEQ ID No: 42, a CDR-H2 domain comprising or consisting of SEQ ID No: 43, a CDR-H3 domain comprising or consisting of SEQ ID No: 44, a CDR-L1 domain comprising or consisting of SEQ ID No: 46, a CDR-L2 domain comprising or consisting of SEQ ID No: 47, and/or a CDR-L3 domain comprising or consisting of SEQ ID No: 48;
(ii) a CDR-H1 domain comprising or consisting of SEQ ID No: 12, a CDR-H2 domain comprising or consisting of SEQ ID No: 13; a CDR-H3 domain comprising or consisting of SEQ ID No: 14, a CDR-L1 domain comprising or consisting of SEQ ID No: 16, a CDR-L2 domain comprising or consisting of SEQ ID No: 17, and/or a CDR-L3 domain comprising or consisting of SEQ ID No: 18;
(iii) a CDR-H1 domain comprising or consisting of SEQ ID No: 22, a CDR-H2 domain comprising or consisting of SEQ ID No: 23; a CDR-H3 domain comprising or consisting of SEQ ID No: 24, a CDR-L1 domain comprising or consisting of SEQ ID No: 26, a CDR-L2 domain comprising or consisting of SEQ ID No: 27, and/or a CDR-L3 domain comprising or consisting of SEQ ID No: 28;
(iv) a CDR-H1 domain comprising or consisting of SEQ ID No: 62, a CDR-H2 domain comprising or consisting of SEQ ID No: 63; a CDR-H3 domain comprising or consisting of SEQ ID No: 64, a CDR-L1 domain comprising or consisting of SEQ ID No: 66, a CDR-L2 domain comprising or consisting of SEQ ID No: 67, and/or a CDR-L3 domain comprising or consisting of SEQ ID No: 68;
(v) a CDR-H1 domain comprising or consisting of SEQ ID No: 32, a CDR-H2 domain comprising or consisting of SEQ ID No: 33; a CDR-H3 domain comprising or consisting of SEQ ID No: 34, a CDR-L1 domain comprising or consisting of SEQ ID No: 36, a CDR-L2 domain comprising or consisting of SEQ ID No: 37, and/or a CDR-L3 domain comprising or consisting of SEQ ID No: 38; or
(vi) a CDR-H1 domain comprising or consisting of SEQ ID No: 52, a CDR-H2 domain comprising or consisting of SEQ ID No: 53; a CDR-H3 domain comprising or consisting of SEQ ID No: 54, a CDR-L1 domain comprising or consisting of SEQ ID No: 56, a CDR-L2 domain comprising or consisting of SEQ ID No: 57, and/or a CDR-L3 domain comprising or consisting of SEQ ID No: 58.

3. The antibody or antigen-binding fragment thereof according to claim 1 or claim 2, wherein the antibody or antigen-binding fragment thereof comprises:
(i) a heavy chain variable region comprising or consisting of SEQ ID No: 45, and a light chain variable region comprising or consisting of SEQ ID No: 49;
(ii) a heavy chain variable region comprising or consisting of SEQ ID No: 15, and a light chain variable region comprising or consisting of SEQ ID No: 19;
(iii) a heavy chain variable region comprising or consisting of SEQ ID No: 25, and a light chain variable region comprising or consisting of SEQ ID No: 29;
(iv) a heavy chain variable region comprising or consisting of SEQ ID No: 65, and a light chain variable region comprising or consisting of SEQ ID No: 69;
(v) a heavy chain variable region comprising or consisting of SEQ ID No: 35, and a light chain variable region comprising or consisting of SEQ ID No: 39; or
(vi) a heavy chain variable region comprising or consisting of SEQ ID No: 55, and a light chain variable region comprising or consisting of SEQ ID No: 59.

4. The antibody or antigen-binding fragment thereof according to any preceding claim, wherein the antibody or antigen-binding fragment thereof comprises:
(i) a heavy chain variable region encoded by a nucleic acid sequence comprising or consisting of SEQ ID No: 78, and a light chain variable region encoded by a nucleic acid sequence comprising or consisting of SEQ ID No: 79;
(ii) a heavy chain variable region encoded by a nucleic acid sequence comprising or consisting of SEQ ID No: 72, and a light chain variable region encoded by a nucleic acid sequence comprising or consisting of SEQ ID No: 73;
(iii) a heavy chain variable region encoded by a nucleic acid sequence comprising or consisting of SEQ ID No: 74, and a light chain variable region encoded by a nucleic acid sequence comprising or consisting of SEQ ID No: 75;
(iv) a heavy chain variable region encoded by a nucleic acid sequence comprising or consisting of SEQ ID No: 82, and a light chain variable region encoded by a nucleic acid sequence comprising or consisting of SEQ ID No: 83;
(v) a heavy chain variable region encoded by a nucleic acid sequence comprising or consisting of SEQ ID No: 76, and a light chain variable region encoded by a nucleic acid sequence comprising or consisting of SEQ ID No: 77; or
(vi) a heavy chain variable region encoded by a nucleic acid sequence comprising or consisting of SEQ ID No: 80, and a light chain variable region encoded by a nucleic acid sequence comprising or consisting of SEQ ID No: 81.

5. The antibody or antigen-binding fragment thereof according to claim 1, wherein the antibody or antigen-binding fragment thereof comprises:
(i) a CDR-H1 domain comprising or consisting of SEQ ID No: 84, a CDR-H2 domain comprising or consisting of SEQ ID No: 85, a CDR-H3 domain comprising or consisting of SEQ ID No: 86, a CDR-L1 domain comprising or consisting of SEQ ID No: 87, a CDR-L2 domain comprising or consisting of SEQ ID No: 88, and/or a CDR-L3 domain comprising or consisting of SEQ ID No: 89; or
(ii) a CDR-H1 domain comprising or consisting of SEQ ID No: 101, a CDR-H2 domain comprising or consisting of SEQ ID No: 102; a CDR-H3 domain comprising or consisting of SEQ ID No: 103, a CDR-L1 domain comprising or consisting of SEQ ID No: 104, a CDR-L2 domain comprising or consisting of SEQ ID No: 105, and/or a CDR-L3 domain comprising or consisting of SEQ ID No: 106.

6. The antibody or antigen-binding fragment thereof according to claim 1 or claim 5, wherein the antibody or antigen-binding fragment thereof comprises:
(i) a heavy chain variable region comprising or consisting of SEQ ID No: 90, and a light chain variable region comprising or consisting of SEQ ID No: 91;
(ii) a heavy chain variable region comprising or consisting of SEQ ID No: 90, and a light chain variable region comprising or consisting of SEQ ID No: 94;
(iii) a heavy chain variable region comprising or consisting of SEQ ID No: 90, and a light chain variable region comprising or consisting of SEQ ID No: 95;
(iv) a heavy chain variable region comprising or consisting of SEQ ID No: 90, and a light chain variable region comprising or consisting of SEQ ID No: 96;
(v) a heavy chain variable region comprising or consisting of SEQ ID No: 97, and a light chain variable region comprising or consisting of SEQ ID No: 91;
(vi) a heavy chain variable region comprising or consisting of SEQ ID No: 97, and a light chain variable region comprising or consisting of SEQ ID No: 94;
(vii) a heavy chain variable region comprising or consisting of SEQ ID No: 97, and a light chain variable region comprising or consisting of SEQ ID No: 95;
(viii) a heavy chain variable region comprising or consisting of SEQ ID No: 97, and a light chain variable region comprising or consisting of SEQ ID No: 96;
(ix) a heavy chain variable region comprising or consisting of SEQ ID No: 98, and a light chain variable region comprising or consisting of SEQ ID No: 91;
(x) a heavy chain variable region comprising or consisting of SEQ ID No: 98, and a light chain variable region comprising or consisting of SEQ ID No: 94;
(xi) a heavy chain variable region comprising or consisting of SEQ ID No: 98, and a light chain variable region comprising or consisting of SEQ ID No: 95;
(xii) a heavy chain variable region comprising or consisting of SEQ ID No: 98, and a light chain variable region comprising or consisting of SEQ ID No: 96;
(xiii) a heavy chain variable region comprising or consisting of SEQ ID No: 99, and a light chain variable region comprising or consisting of SEQ ID No: 91;
(xiv) a heavy chain variable region comprising or consisting of SEQ ID No: 99, and a light chain variable region comprising or consisting of SEQ ID No: 94;
(xv) a heavy chain variable region comprising or consisting of SEQ ID No: 99, and a light chain variable region comprising or consisting of SEQ ID No: 95;
(xvi) a heavy chain variable region comprising or consisting of SEQ ID No: 99, and a light chain variable region comprising or consisting of SEQ ID No: 96;
(xvii) a heavy chain variable region comprising or consisting of SEQ ID No: 100, and a light chain variable region comprising or consisting of SEQ ID No: 91;
(xviii) a heavy chain variable region comprising or consisting of SEQ ID No: 100, and a light chain variable region comprising or consisting of SEQ ID No: 94;
(xix) a heavy chain variable region comprising or consisting of SEQ ID No: 100, and a light chain variable region comprising or consisting of SEQ ID No: 95;
(xx) a heavy chain variable region comprising or consisting of SEQ ID No: 100, and a light chain variable region comprising or consisting of SEQ ID No: 96;
(xxi) a heavy chain variable region comprising or consisting of SEQ ID No: 107, and a light chain variable region comprising or consisting of SEQ ID No: 108;
(xxii) a heavy chain variable region comprising or consisting of SEQ ID No: 107, and a light chain variable region comprising or consisting of SEQ ID No: 109;
(xxiii) a heavy chain variable region comprising or consisting of SEQ ID No: 107, and a light chain variable region comprising or consisting of SEQ ID No: 110;
(xxiv) a heavy chain variable region comprising or consisting of SEQ ID No: 107, and a light chain variable region comprising or consisting of SEQ ID No: 111;
(xxv) a heavy chain variable region comprising or consisting of SEQ ID No: 112, and a light chain variable region comprising or consisting of SEQ ID No: 108;
(xxvi) a heavy chain variable region comprising or consisting of SEQ ID No: 112, and a light chain variable region comprising or consisting of SEQ ID No: 109;
(xxvii) a heavy chain variable region comprising or consisting of SEQ ID No: 112, and a light chain variable region comprising or consisting of SEQ ID No: 110;
(xxviii) a heavy chain variable region comprising or consisting of SEQ ID No: 112, and a light chain variable region comprising or consisting of SEQ ID No: 111;
(xxix) a heavy chain variable region comprising or consisting of SEQ ID No: 113, and a light chain variable region comprising or consisting of SEQ ID No: 108;
(xxx) a heavy chain variable region comprising or consisting of SEQ ID No: 113, and a light chain variable region comprising or consisting of SEQ ID No: 109;
(xxxi) a heavy chain variable region comprising or consisting of SEQ ID No: 113, and a light chain variable region comprising or consisting of SEQ ID No: 110;
(xxxii) a heavy chain variable region comprising or consisting of SEQ ID No: 113, and a light chain variable region comprising or consisting of SEQ ID No: 111;
(xxxiii) a heavy chain variable region comprising or consisting of SEQ ID No: 114, and a light chain variable region comprising or consisting of SEQ ID No: 108;
(xxxiv) a heavy chain variable region comprising or consisting of SEQ ID No: 114, and a light chain variable region comprising or consisting of SEQ ID No: 109;
(xxxv) a heavy chain variable region comprising or consisting of SEQ ID No: 114, and a light chain variable region comprising or consisting of SEQ ID No: 110; or
(xxxvi) a heavy chain variable region comprising or consisting of SEQ ID No: 114, and a light chain variable region comprising or consisting of SEQ ID No: 111.

7. The antibody or antigen-binding fragment thereof according to any preceding claim, wherein the antibody or antigen-binding fragment thereof comprises a disabled Fc fragment, optionally wherein the disabled Fc fragment comprises one or more amino acid substitution selected from the group consisting of: L234A, L235A, and P329G.

8. An antibody or antigen-binding fragment thereof according to any one of the preceding claims, for use in therapy.

9. An antibody or antigen-binding fragment thereof according to any one of claims 1 to 7, for use in treating, preventing or ameliorating a condition caused by platelet-mediated aggregation.

10. An antibody or antigen-binding fragment thereof for use according to claim 9, wherein the condition caused by platelet-mediated aggregation may be selected from the group consisting of: a thrombotic-related condition; thrombotic thrombocytopenic purpura (TTP) (also referred to as acquired thrombotic thrombocytopeniarpura (aTTP) or immune thrombotic thrombocytopenia purpura (iTTP) or congenital thrombotic thrombocytopenia purpura (cTTP)), acute coronary syndrome (ACS), atherosclerosis, ischemic stroke, atrial fibrillation (AF), acute myocardial infarction (AMI), cardiovascular disease (CVD), thrombosis, unstable angina, stable angina, angina pectoris, embolus formation, deep vein thrombosis, haemolytic uremic syndrome, haemolytic anaemia, acute renal failure, thrombolytic complications, disseminated intravascular coagulation, coronary heart disease, thromboembolic complications, restenosis, chronic unstable angina, peripheral vascular disease, arterial thrombosis, pre-eclampsia, embolism, restenosis, sepsis, vascular inflammation, glomerulonephritis, and thrombotic condition resulting from a coronavirus infection.

11. A pharmaceutical composition comprising an antibody or antigen-binding fragment thereof according to any one of claims 1 to 7, and optionally a pharmaceutically acceptable vehicle.

12. A process for making the pharmaceutical composition according to claim 11, the process comprising combining a therapeutically effective amount of an antibody or antigen-binding fragment thereof according to any one of claims 1 to 7, with a pharmaceutically acceptable vehicle.

13. A polynucleotide sequence encoding the antibody, or antigen-binding fragment thereof as defined in any one of claims 1 to 7.

14. An expression cassette comprising a polynucleotide sequence according to claim 13.

15. A recombinant vector comprising the expression cassette according claim 14.

16. A host cell comprising the polynucleotide sequence according to claim 13, the expression cassette according to claim 14, or the vector according to claim 15.

17. A method of preparing the antibody, or antigen-binding fragment thereof according to any one of claims 1 to 7, the method comprising:
a) introducing, into a host cell, the vector of claim 15; and
b) culturing the host cell under conditions to result in the production of the antibody, or antigen-binding fragment thereof according to any one of claims 1 to 7.

18. The antibody or antigen-binding fragment thereof according to any one of claims 1 to 7, for use in diagnosis or prognosis.

19. The antibody or antigen-binding fragment thereof according to any one of claims 1 to 7, for use in diagnosing or prognosing a condition caused by platelet-mediated aggregation.

20. A method of diagnosing or prognosing a condition caused by platelet-mediated aggregation in a subject, the method comprising detecting VWF in a biological sample obtained from the subject with the antibody or antigen-binding fragment thereof according to any one of claims 1 to 7.

21. A kit for diagnosing a subject suffering from a condition caused by platelet-mediated aggregation, or for providing a prognosis of the subject's condition, the kit comprising an antibody or antigen-binding fragment thereof according to any one of claims 1 to 7 for detecting VWF in a sample from a test subject.

22. The use according to claim 19, the method according to claim 20, or the kit according to claim 21, wherein the condition caused by platelet-mediated aggregation may be selected from the group consisting of: a thrombotic-related condition; thrombotic thrombocytopenia purpura (TTP) (also referred to as acquired thrombotic thrombocytopenia purpura (aTTP) or immune thrombotic thrombocytopeniaurpura (iTTP) or congenital thrombotic thrombocytopenia purpura (cTTP)), acute coronary syndrome (ACS), atherosclerosis, ischemic stroke, atrial fibrillation (AF), acute myocardial infarction (AMI), cardiovascular disease (CVD), thrombosis, unstable angina, stable angina, angina pectoris, embolus formation, deep vein thrombosis, haemolytic uremic syndrome, haemolytic anaemia, acute renal failure, thrombolytic complications, disseminated intravascular coagulation, coronary heart disease, thromboembolic complications, restenosis, chronic unstable angina, peripheral vascular disease, arterial thrombosis, pre-eclampsia, embolism, restenosis, sepsis, vascular inflammation, glomerulonephritis, and thrombotic condition resulting from a coronavirus infection.


SEQUENCE LISTING (114 sequences)

SEQ ID NO: 1 - protein, 2813 aa, Homo sapiens
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCQDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 2 - protein, 468 aa, Homo sapiens
TQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEP

SEQ ID NO: 3 - protein, 79 aa, Homo sapiens
TQCIGEDGVQHQFLEAWVPDHQPCQICTCLSGRKVNCTTQPCPTAKAPTCGLCEVARLRQNADQCCPEYECVCDPVSCD

SEQ ID NO: 4 - protein, 69 aa, Homo sapiens
LPPVPHCERGLQPTLTNPGECRPNFTCACRKEECKRVSPPSCPPHRLPTLRKTQCCDEYECACNCVNST

SEQ ID NO: 5 - protein, 67 aa, Homo sapiens
VCVHRSTIYPVGQFWEEGCDVCTCTDMEDAVMGLRVAQCSQKPCEDSCRSGFTYVLHEGECCGRCLP

SEQ ID NO: 6 - protein, 81 aa, Homo sapiens
SACEVVTGSPRGDSQSSWKSVGSQWASPENPCLINECVRVKEEVFIQQRNVSCPQLEVPVCPSGFQLSCKTSACCPSCRCE

SEQ ID NO: 7 - protein, 69 aa, Homo sapiens
RMEACMLNGTVIGPGKTVMIDVCTTCRCMVQVGVISGFKLECRKTTCNPCPLGYKEENNTGECCGRCLP

SEQ ID NO: 8 - protein, 76 aa, Homo sapiens
TACTIQLRGGQIMTLKRDETLQDGCDTHFCKVNERGEYFWEKRVTGCPPFDEHKCLAEGGKIMKIPGTCCDTCEEP

SEQ ID NO: 9 - protein, 177 aa, Homo sapiens
DLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEI

SEQ ID NO: 10 - protein, 172 aa, Homo sapiens
DVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVQRCC

SEQ ID NO: 11 - protein, 181 aa, Homo sapiens
DVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKL

SEQ ID NO: 12 - protein, 8 aa, synthetic construct
GYTFTDYF

SEQ ID NO: 13 - protein, 8 aa, synthetic construct
INPHNGGT

SEQ ID NO: 14 - protein, 9 aa, synthetic construct
ARRPLGSDY

SEQ ID NO: 15 - protein, 116 aa, synthetic construct
EVQLQQSGPVLVKPGASVKMSCKASGYTFTDYFMNWVKQSHGKSLEWIGVINPHNGGTSYNQKFKGKATLTVDKSSSTAYMELNSLTSEDSAVYYCARRPLGSDYWGQGTTLTVSS

SEQ ID NO: 16 - protein, 6 aa, synthetic construct
QNVGTA

SEQ ID NO: 17 - skipped

SEQ ID NO: 18 - protein, 9 aa, synthetic construct
QQYSSYPLT

SEQ ID NO: 19 - protein, 107 aa, synthetic construct
DIVMTQSQKFMSTTVGDRVSITCKASQNVGTAVAWYQQKPGQSPKLLIYSASSRYTEVPDRFTGSGSGTDFTLTISYMQSEDLADYFCQQYSSYPLTFGAGTKLELR

SEQ ID NO: 20 - protein, 330 aa, synthetic construct [sequence omitted]

SEQ ID NO: 21 - protein, 106 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 8 aa, synthetic construct
GFNIIHTY

SEQ ID NO: 23 - protein, 8 aa, synthetic construct
VDPTNGYT

SEQ ID NO: 24 - protein, 12 aa, synthetic construct
TREEPSAVVPDD

SEQ ID NO: 25 - protein, 119 aa, synthetic construct
EVQLQQSVAELVRPGASVKLSCTASGFNIIHTYMHWVKQRPEQGLEWIGRVDPTNGYTKYAPKFQGKAAITADTSSNTAYLQLSGLTSEDTAIYYCTREEPSAVVPDDWGQGTTLTVSS

SEQ ID NO: 26 - protein, 6 aa, synthetic construct
QNVRTA

SEQ ID NO: 27 - skipped

SEQ ID NO: 28 - protein, 9 aa, synthetic construct
LQHWNYPLT

SEQ ID NO: 29 - protein, 107 aa, synthetic construct
DIVMTQSQKFMSTSVGDRVSITCRASQNVRTAVTWYQQKPGQSPQALIYLTSNRHTGVPDRFTGSGSGTDFTLTINNVQSEDLADYFCLQHWNYPLTFGGGTKLEIK

SEQ ID NO: 30 - protein, 330 aa, synthetic construct [sequence omitted]

SEQ ID NO: 31 - protein, 106 aa, synthetic construct; identical to SEQ ID NO: 21

SEQ ID NO: 32 - protein, 8 aa, synthetic construct
GYTFTDHT

SEQ ID NO: 33 - protein, 8 aa, synthetic construct
IYPRDGTT

SEQ ID NO: 34 - protein, 15 aa, synthetic construct
SRWAFYYGSSPGFDV

SEQ ID NO: 35 - protein, 122 aa, synthetic construct
QVQLQQSDADLVKPGASVKISCKVSGYTFTDHTIHWMKQRPEQGLEWIGYIYPRDGTTKYNEKFKGKATLTADKSSSTAYMQLNSLTSEDSAVYFCSRWAFYYGSSPGFDVWGTGTSVTVSS

SEQ ID NO: 36 - protein, 11 aa, synthetic construct
KSLLYSNGNTY

SEQ ID NO: 37 - skipped

SEQ ID NO: 38 - protein, 9 aa, synthetic construct
MQHLEYPFT

SEQ ID NO: 39 - protein, 112 aa, synthetic construct
DIVMTQAAPSVPVTPGESVSISCRSSKSLLYSNGNTYLYWFLQRPGQSPQVLIYRMSNLASGVPDRFSGSGSGTAFTLRISRVEAEDVGVYYCMQHLEYPFTFGAGTKLELK

SEQ ID NO: 40 - protein, 330 aa, synthetic construct [sequence omitted]

SEQ ID NO: 41 - protein, 106 aa, synthetic construct; identical to SEQ ID NO: 21

SEQ ID NO: 42 - protein, 8 aa, synthetic construct
GYTFTDYY

SEQ ID NO: 43 - protein, 8 aa, synthetic construct
INPYNGGT

SEQ ID NO: 44 - protein, 11 aa, synthetic construct
ASEGLRTWFTY

SEQ ID NO: 45 - protein, 118 aa, synthetic construct
EVQLQQSRPVLVKPGASVKMSCKASGYTFTDYYLNWVKQSPGKRLEWIGIINPYNGGTNYNQRFKGKATLTVDKSSSTAYMELNSLTSEDSAVYFCASEGLRTWFTYWGQGTLVTVSA

SEQ ID NO: 46 - protein, 12 aa, synthetic construct
QSLSNSGNQKIY

SEQ ID NO: 47 - skipped

SEQ ID NO: 48 - protein, 9 aa, synthetic construct
QNDHSHPFT

SEQ ID NO: 49 - protein, 113 aa, synthetic construct
DIVMTQSPSSLSVSAGEKVTMSCKSSQSLSNSGNQKIYLAWYQQKPGQPPKLLIYGASSRESGVPDRFTGSGSEIDFTLTISSVQAEDLAVYYCQNDHSHPFTFGSGTKLEIK

SEQ ID NO: 50 - protein, 330 aa, synthetic construct [sequence omitted]

SEQ ID NO: 51 - protein, 106 aa, synthetic construct; identical to SEQ ID NO: 21

SEQ ID NO: 52 - protein, 8 aa, synthetic construct
GYTFTDYR

SEQ ID NO: 53 - protein, 8 aa, synthetic construct
IIPNNGDT

SEQ ID NO: 54 - protein, 16 aa, synthetic construct
ARRGYFSYDGGYTMDY

SEQ ID NO: 55 - protein, 123 aa, synthetic construct
EVQLQQSGPELVKPGASVKMSCKASGYTFTDYRLKWVKQSHGKSLEWIGDIIPNNGDTFYNQRFKGKATLTVDKSSNTAYMHLNSLTSEDSAVYYCARRGYFSYDGGYTMDYWGQGTSVTVSS

SEQ ID NO: 56 - protein, 11 aa, synthetic construct
QSLVHSNGNTY

SEQ ID NO: 57 - skipped

SEQ ID NO: 58 - protein, 9 aa, synthetic construct
SQSTHVPWT

SEQ ID NO: 59 - protein, 112 aa, synthetic construct
DDVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 60 - protein, 330 aa, synthetic construct [sequence omitted]

SEQ ID NO: 61 - protein, 106 aa, synthetic construct; identical to SEQ ID NO: 21

SEQ ID NO: 62 - protein, 8 aa, synthetic construct
GFSLTSYG

SEQ ID NO: 63 - protein, 7 aa, synthetic construct
IWGDGST

SEQ ID NO: 64 - protein, 13 aa, synthetic construct
AKEPYYGSPYFDV

SEQ ID NO: 65 - protein, 119 aa, synthetic construct
QVQLKESGPGLVAPSQSLSITCTVSGFSLTSYGVNWVRQPPGKGLEWLGVIWGDGSTNYHSALISRVSISKDDSKSQVFLKLNSLQTDDTATYYCAKEPYYGSPYFDVWGAGTTVTVSS

SEQ ID NO: 66 - protein, 5 aa, synthetic construct
SSVSY

SEQ ID NO: 67 - skipped

SEQ ID NO: 68 - protein, 9 aa, synthetic construct
QQWSSNPPT

SEQ ID NO: 69 - protein, 106 aa, synthetic construct
QIVLTQSPAIMSTSPGEKVTLTCSASSSVSYMHWYQQKSGTSPKRWIYDTSKLASGVPARFSGSGSGTSYSLTLSSMEAEDAATYYCQQWSSNPPTFGGGTKLEIK

SEQ ID NO: 70 - protein, 330 aa, synthetic construct [sequence omitted]

SEQ ID NO: 71 - protein, 106 aa, synthetic construct; identical to SEQ ID NO: 21

SEQ ID NO: 72 - DNA, 348 bp, synthetic construct
  source 1..348: mol_type = other DNA
gaggtccagctgcaacagtctggacctgtgctggtgaagcctggggcttcagtgaagatgtcctgtaaggcttctggatacacattcactgactactttatgaactgggtgaagcagagccatggaaagagccttgagtggattggagttattaatcctcacaacggtggtactagctataaccagaagttcaagggcaaggccacattgactgttgacaagtcctccagcacagcctacatggagctcaacagcctgacatctgaggactctgcagtctattactgtgcaagaaggcccctggggtctgactactggggccaaggcaccactctcacagtctcctca

SEQ ID NO: 73 - DNA, 321 bp, synthetic construct
  source 1..321: mol_type = other DNA
gacattgtgatgacccagtctcaaaaattcatgtccacaacagtaggagacagggtcagtatcacctgcaaggccagtcagaatgtgggtactgctgtagcctggtatcaacagaaaccaggacaatctcctaaactactgatttactcagcatccagtcggtacactgaagtccctgatcgcttcacaggcagtggatctgggacagatttcactctcaccattagctatatgcagtctgaagacctggcagattatttctgtcagcaatatagcagctatcctctcacgttcggtgctgggaccaagctggagctgaga

SEQ ID NO: 74 - DNA, 357 bp, synthetic construct
  source 1..357: mol_type = other DNA
gaggttcagctgcagcagtctgtggcagaacttgtgaggccaggggcctcagtcaagttgtcctgcacagcttctggcttcaacattatacacacctatatgcactgggtgaagcagaggcctgaacagggcctggagtggattggaagggttgatcctactaatggttatactaaatatgccccgaagttccagggcaaggccgctataactgcagacacatcctccaatacagcctacctgcagctcagcggcctgacatctgaggacactgccatctattactgtactagggaagaacctagtgccgtagtcccagacgactggggccaaggcaccactctcacagtctcctca

SEQ ID NO: 75 - DNA, 321 bp, synthetic construct
  source 1..321: mol_type = other DNA
gacattgtgatgacccagtctcaaaaattcatgtccacatcagtaggagacagggtcagcatcacctgcagggccagtcagaatgttcgtactgctgtaacctggtatcaacagaaaccagggcagtctcctcaagcactgatttacttgacatccaaccggcacactggagtccctgatcgcttcacaggcagtggatctgggacagatttcactctcaccattaacaatgtgcaatctgaagacctggcagattatttctgtctgcaacattggaattatcctctaacgttcggaggggggaccaagctggaaataaaa

SEQ ID NO: 76 - DNA, 366 bp, synthetic construct
  source 1..366: mol_type = other DNA
caggttcagctgcaacagtctgacgctgacttggtgaaacctggagcttcagtgaagatatcctgcaaggtttctggctacaccttcactgaccatactattcactggatgaagcagaggcctgaacagggcctggaatggattggatatatttatcctagagatggtactactaagtacaatgagaagttcaagggcaaggccacattgactgcagacaaatcctccagcacagcctacatgcagctcaacagcctgacatctgaggactctgcagtctatttctgttcaagatgggcgttttactacggtagtagccccggcttcgatgtctggggcacagggacctcggtcaccgtctcctca

SEQ ID NO: 77 - DNA, 336 bp, synthetic construct
  source 1..336: mol_type = other DNA
gatattgtgatgactcaggctgcaccctctgtacctgtcactcctggagagtcagtatccatctcctgcaggtctagtaagagtctcctgtatagtaatggcaacacttacttgtattggttcctgcagaggccaggccagtctcctcaggtcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatcctttcacgttcggtgctgggaccaagctggagctgaaa

SEQ ID NO: 78 - DNA, 354 bp, synthetic construct
  source 1..354: mol_type = other DNA
gaggtccagctgcaacagtctagacctgtgctggtgaagcctggggcttcagtgaagatgtcctgtaaggcttctggatacacattcactgactattatctgaactgggtgaagcagagccctggaaagaggcttgagtggattggaattattaatccttacaacggtggtactaactacaaccagaggttcaagggcaaggccacattgactgttgacaagtcctccagcacagcctacatggaactcaacagcctgacatctgaggactctgcagtctatttctgtgcaagtgagggattacggacttggtttacttactggggccaagggactctggtcactgtctctgca

SEQ ID NO: 79 - DNA, 339 bp, synthetic construct
  source 1..339: mol_type = other DNA
gacattgtgatgacacagtctccatcctccctgagtgtgtcagcaggagagaaggtcactatgagctgcaagtccagtcagagtctgtcaaatagtggaaatcaaaagatctacttggcctggtaccagcagaaaccagggcagcctcctaaattgttgatttatggggcatccagcagggaatctggggtccctgatcgcttcacaggcagtggatctgaaatcgatttcactcttaccatcagcagtgtgcaggctgaagacctggcagtttattactgtcagaatgatcatagtcatccattcacgttcggctcggggacaaagttggaaataaaa

SEQ ID NO: 80 - DNA, 369 bp, synthetic construct
  source 1..369: mol_type = other DNA
gaggtccagctgcaacagtctggacctgagctggtgaagcctggggcttcagtgaagatgtcctgcaaggcttctggatacaccttcactgactaccgcttgaagtgggtgaagcagagccatggaaagagccttgagtggattggagatattattcctaacaatggtgatactttttacaaccagaggttcaagggcaaggccacattgactgtagacaaatcctccaacacagcctacatgcacctcaacagcctgacatctgaggactctgcagtctattactgtgcaagaaggggctactttagctacgacgggggctatactatggactactggggtcagggaacctcagtcaccgtctcctca

SEQ ID NO: 81 - DNA, 336 bp, synthetic construct
  source 1..336: mol_type = other DNA
gatgatgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 82 - DNA, 357 bp, synthetic construct
  source 1..357: mol_type = other DNA
caggtgcagctgaaggagtcaggacctggcctggtggcgccttcacagagcctgtccatcacatgcactgtctcagggttctcattaaccagttatggtgtaaactgggttcgccagcctccaggaaagggtctggagtggctgggagtaatatggggtgacgggagcacaaattatcattcagctctcatatccagagtgagcatcagcaaggatgactccaagagccaagttttcttaaaactgaacagtctgcaaactgatgacacagccacgtactactgtgccaaagagccttactacggtagtccgtacttcgatgtctggggcgcagggaccacggtcaccgtctcctca

SEQ ID NO: 83 - DNA, 318 bp, synthetic construct
  source 1..318: mol_type = other DNA
caaattgttctcacccagtctccagcaatcatgtctacatctccaggggagaaggtcaccctgacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacactcagcagcatggaggctgaagatgctgccacttattactgccagcagtggagtagtaacccacccacgttcggaggggggaccaagctggaaataaaa

SEQ ID NO: 84 - protein, 8 aa, synthetic construct
GYTFTDYY

SEQ ID NO: 85 - protein, 8 aa, synthetic construct
INPYNGGT

SEQ ID NO: 86 - protein, 11 aa, synthetic construct
ASEGLRTWFTY

SEQ ID NO: 87 - protein, 12 aa, synthetic construct
QSLSNSGNQKIY

SEQ ID NO: 88 - skipped

SEQ ID NO: 89 - protein, 9 aa, synthetic construct
QNDHSHPFT

SEQ ID NO: 90 - protein, 118 aa, synthetic construct
QVQLVQSGAEVKKPGASVKVSCKASGYTFTDYYLNWVRQAPGQRLEWMGIINPYNGGTNYSQKFQGRVTITRDTSASTAYMELSSLRSEDTAVYYCASEGLRTWFTYWGQGTLVTVSS

SEQ ID NO: 91 - protein, 113 aa, synthetic construct
DIVMTQSPDSLAVSLGERATINCKSSQSLSNSGNQKIYLAWYQQKPGQPPKLLIYGASSRESGVPDRFSGSGSGTDFTLTISSLQAEDVAVYYCQNDHSHPFTFGQGTKLEIK

SEQ ID NO: 92 - protein, 330 aa, synthetic construct [sequence omitted]

SEQ ID NO: 93 - protein, 106 aa, synthetic construct; identical to SEQ ID NO: 21

SEQ ID NO: 94 - protein, 113 aa, synthetic construct
DIVMTQSPDSLAVSLGERATISCKSSQSLSNSGNQKIYLAWYQQKPGQPPKLLIYGASSRESGVPDRFSGSGSGTDFTLTISSLQAEDVAVYYCQNDHSHPFTFGQGTKLEIK

SEQ ID NO: 95 - protein, 113 aa, synthetic construct
DIVMTQSPDSLAVSLGERATINCKSSQSLSNSGNQKIYLAWYQQKPGQPPKLLIYGASSRESGVPDRFSGSGSEIDFTLTISSLQAEDVAVYYCQNDHSHPFTFGQGTKLEIK

SEQ ID NO: 96 - protein, 113 aa, synthetic construct
DIVMTQSPDSLAVSLGERATISCKSSQSLSNSGNQKIYLAWYQQKPGQPPKLLIYGASSRESGVPDRFSGSGSEIDFTLTISSLQAEDVAVYYCQNDHSHPFTFGQGTKLEIK

SEQ ID NO: 97 - protein, 118 aa, synthetic construct
QVQLVQSGAEVKKPGASVKVSCKASGYTFTDYYLNWVRQAPGQRLEWMGIINPYNGGTNYNQRFKGRVTITRDTSASTAYMELSSLRSEDTAVYYCASEGLRTWFTYWGQGTLVTVSS

SEQ ID NO: 98 - protein, 118 aa, synthetic construct
QVQLVQSGAEVKKPGASVKVSCKASGYTFTDYYLNWVRQAPGQRLEWMGIINPYNGGTNYSQKFQGRVTITVDKSSSTAYMELSSLRSEDTAVYYCASEGLRTWFTYWGQGTLVTVSS

SEQ ID NO: 99 - protein, 118 aa, synthetic construct
QVQLVQSGAEVKKPGASVKVSCKASGYTFTDYYLNWVRQAPGQRLEWMGIINPYNGGTNYNQRFKGRVTITVDKSSSTAYMELSSLRSEDTAVYYCASEGLRTWFTYWGQGTLVTVSS

SEQ ID NO: 100 - protein, 118 aa, synthetic construct
EVQLVQSGAEVKKPGASVKVSCKASGYTFTDYYLNWVRQAPGQRLEWMGIINPYNGGTNYNQRFKGRVTLTVDKSSSTAYMELSSLRSEDTAVYYCASEGLRTWFTYWGQGTLVTVSS

SEQ ID NO: 101 - protein, 8 aa, synthetic construct
GYTFTDYF

SEQ ID NO: 102 - protein, 8 aa, synthetic construct
INPHNGGT

SEQ ID NO: 103 - protein, 9 aa, synthetic construct
ARRPLGSDY

SEQ ID NO: 104 - protein, 6 aa, synthetic construct
QNVGTA

SEQ ID NO: 105 - skipped

SEQ ID NO: 106 - protein, 9 aa, synthetic construct
QQYSSYPLT

SEQ ID NO: 107 - protein, 116 aa, synthetic construct
QVQLVQSGAEVKKPGASVKVSCKASGYTFTDYFMNWVRQAPGQRLEWMGVINPHNGGTSYSQKFQGRVTITRDTSASTAYMELSSLRSEDTAVYYCARRPLGSDYWGQGTTVTVSS

SEQ ID NO: 108 - protein, 107 aa, synthetic construct
DIQMTQSPSSVSASVGDRVTITCKASQNVGTAVAWYQQKPGKAPKLLIYSASSRYTGVPSRFSGSGSGTDFTLTISSLQPEDFATYYCQQYSSYPLTFGQGTKLEIK

SEQ ID NO: 109 - protein, 107 aa, synthetic construct
DIVMTQSPSSVSASVGDRVTITCKASQNVGTAVAWYQQKPGKAPKLLIYSASSRYTGVPSRFSGSGSGTDFTLTISSLQPEDFATYYCQQYSSYPLTFGQGTKLEIK

SEQ ID NO: 110 - protein, 107 aa, synthetic construct
DIQMTQSPSSVSASVGDRVTITCKASQNVGTAVAWYQQKPGKAPKLLIYSASSRYTEVPDRFSGSGSGTDFTLTISSLQPEDFATYYCQQYSSYPLTFGQGTKLEIK

SEQ ID NO: 111 - protein, 107 aa, synthetic construct
DIVMTQSPSSVSASVGDRVTITCKASQNVGTAVAWYQQKPGKAPKLLIYSASSRYTEVPDRFSGSGSGTDFTLTISSLQPEDFATYYCQQYSSYPLTFGQGTKLEIK

SEQ ID NO: 112 - protein, 116 aa, synthetic construct
QVQLVQSGAEVKKPGASVKVSCKASGYTFTDYFMNWVRQAPGQRLEWMGVINPHNGGTSYSQKFQGRVTITVDKSSSTAYMELSSLRSEDTAVYYCARRPLGSDYWGQGTTVTVSS

SEQ ID NO: 113 - protein, 116 aa, synthetic construct
EVQLVQSGAEVKKPGASVKVSCKASGYTFTDYFMNWVRQAPGQRLEWMGVINPHNGGTSYSQKFQGRVTITVDKSSSTAYMELSSLRSEDTAVYYCARRPLGSDYWGQGTTVTVSS

SEQ ID NO: 114 - protein, 116 aa, synthetic construct
EVQLVQSGAEVKKPGASVKVSCKASGYTFTDYFMNWVRQAPGQRLEWMGVINPHNGGTSYSQKFQGRVTLTVDKSSSTAYMELSSLRSEDTAVYYCARRPLGSDYWGQGTTVTVSS